SEQUENCE LISTING (386 sequences)

SEQ ID NO: 1 - protein, 12 aa, Artificial Sequence
  Synthetic polypeptide
ILGTILGLLKSL

SEQ ID NO: 2 - protein, 12 aa, Artificial Sequence
  Synthetic polypeptide
ALGTILGLLKSL

SEQ ID NO: 3 - protein, 12 aa, Artificial Sequence
  Synthetic polypeptide
IAGTILGLLKSL

SEQ ID NO: 4 - protein, 12 aa, Artificial Sequence
  Synthetic polypeptide
ILATILGLLKSL

SEQ ID NO: 5 - protein, 12 aa, Artificial Sequence
  Synthetic polypeptide
ILGAILGLLKSL

SEQ ID NO: 6 - protein, 12 aa, Artificial Sequence
  Synthetic polypeptide
ILGTALGLLKSL

SEQ ID NO: 7 - protein, 12 aa, Artificial Sequence
  Synthetic polypeptide
ILGTIAGLLKSL

SEQ ID NO: 8 - protein, 12 aa, Artificial Sequence
  Synthetic polypeptide
ILGTILALLKSL

SEQ ID NO: 9 - protein, 12 aa, Artificial Sequence
  Synthetic polypeptide
ILGTILGALKSL

SEQ ID NO: 10 - protein, 12 aa, Artificial Sequence
  Synthetic polypeptide
ILGTILGLAKSL

SEQ ID NO: 11 - protein, 12 aa, Artificial Sequence
  Synthetic polypeptide
ILGTILGLLASL

SEQ ID NO: 12 - protein, 12 aa, Artificial Sequence
  Synthetic polypeptide
ILGTILGLLKAL

SEQ ID NO: 13 - protein, 12 aa, Artificial Sequence
  Synthetic polypeptide
ILGTILGLLKSA

SEQ ID NO: 14 - protein, 12 aa, Artificial Sequence
  Synthetic polypeptide
ILGTLLGLKKSL

SEQ ID NO: 15 - protein, 12 aa, Artificial Sequence
  Synthetic polypeptide
ILGTKLGLLKSL

SEQ ID NO: 16 - protein, 12 aa, Artificial Sequence
  Synthetic polypeptide
ILGKILGLLKSL

SEQ ID NO: 17 - protein, 12 aa, Artificial Sequence
  Synthetic polypeptide
ILGTILKLLKSL

SEQ ID NO: 18 - protein, 12 aa, Artificial Sequence
  Synthetic polypeptide
ILGTILGLFKSL

SEQ ID NO: 19 - protein, 11 aa, Artificial Sequence
  Synthetic polypeptide
ILGTILGLLKS

SEQ ID NO: 20 - protein, 12 aa, Artificial Sequence
  Synthetic polypeptide
ILETKLGLLKSE

SEQ ID NO: 21 - protein, 12 aa, Artificial Sequence
  Synthetic polypeptide
GLGTILGLLKSL

SEQ ID NO: 22 - protein, 11 aa, Artificial Sequence
  Synthetic polypeptide
SLLSLIRKLLT

SEQ ID NO: 23 - protein, 12 aa, Artificial Sequence
  Synthetic polypeptide
ALGTILKLLKSL

SEQ ID NO: 24 - protein, 12 aa, Artificial Sequence
  Synthetic polypeptide
CLGTILKLLKSL

SEQ ID NO: 25 - protein, 12 aa, Artificial Sequence
  Synthetic polypeptide
DLGTILKLLKSL

SEQ ID NO: 26 - protein, 12 aa, Artificial Sequence
  Synthetic polypeptide
ELGTILKLLKSL

SEQ ID NO: 27 - protein, 12 aa, Artificial Sequence
  Synthetic polypeptide
FLGTILKLLKSL

SEQ ID NO: 28 - protein, 12 aa, Artificial Sequence
  Synthetic polypeptide
GLGTILKLLKSL

SEQ ID NO: 29 - protein, 12 aa, Artificial Sequence
  Synthetic polypeptide
HLGTILKLLKSL

SEQ ID NO: 30 - protein, 12 aa, Artificial Sequence
  Synthetic polypeptide
KLGTILKLLKSL

SEQ ID NO: 31 - protein, 12 aa, Artificial Sequence
  Synthetic polypeptide
LLGTILKLLKSL

SEQ ID NO: 32 - protein, 12 aa, Artificial Sequence
  Synthetic polypeptide
MLGTILKLLKSL

SEQ ID NO: 33 - protein, 12 aa, Artificial Sequence
  Synthetic polypeptide
NLGTILKLLKSL

SEQ ID NO: 34 - protein, 12 aa, Artificial Sequence
  Synthetic polypeptide
PLGTILKLLKSL

SEQ ID NO: 35 - protein, 12 aa, Artificial Sequence
  Synthetic polypeptide
QLGTILKLLKSL

SEQ ID NO: 36 - protein, 12 aa, Artificial Sequence
  Synthetic polypeptide
RLGTILKLLKSL

SEQ ID NO: 37 - protein, 12 aa, Artificial Sequence
  Synthetic polypeptide
SLGTILKLLKSL

SEQ ID NO: 38 - protein, 12 aa, Artificial Sequence
  Synthetic polypeptide
TLGTILKLLKSL

SEQ ID NO: 39 - protein, 12 aa, Artificial Sequence
  Synthetic polypeptide
VLGTILKLLKSL

SEQ ID NO: 40 - protein, 12 aa, Artificial Sequence
  Synthetic polypeptide
WLGTILKLLKSL

SEQ ID NO: 41 - protein, 12 aa, Artificial Sequence
  Synthetic polypeptide
YLGTILKLLKSL

SEQ ID NO: 42 - protein, 12 aa, Artificial Sequence
  Synthetic polypeptide
IAGTILKLLKSL

SEQ ID NO: 43 - protein, 12 aa, Artificial Sequence
  Synthetic polypeptide
ICGTILKLLKSL

SEQ ID NO: 44 - protein, 12 aa, Artificial Sequence
  Synthetic polypeptide
IDGTILKLLKSL

SEQ ID NO: 45 - protein, 12 aa, Artificial Sequence
  Synthetic polypeptide
IEGTILKLLKSL

SEQ ID NO: 46 - protein, 12 aa, Artificial Sequence
  Synthetic polypeptide
IFGTILKLLKSL

SEQ ID NO: 47 - protein, 12 aa, Artificial Sequence
  Synthetic polypeptide
IGGTILKLLKSL

SEQ ID NO: 48 - protein, 12 aa, Artificial Sequence
  Synthetic polypeptide
IHGTILKLLKSL

SEQ ID NO: 49 - protein, 12 aa, Artificial Sequence
  Synthetic polypeptide
IIGTILKLLKSL

SEQ ID NO: 50 - protein, 12 aa, Artificial Sequence
  Synthetic polypeptide
IKGTILKLLKSL

SEQ ID NO: 51 - protein, 12 aa, Artificial Sequence
  Synthetic polypeptide
IMGTILKLLKSL

SEQ ID NO: 52 - protein, 12 aa, Artificial Sequence
  Synthetic polypeptide
INGTILKLLKSL

SEQ ID NO: 53 - protein, 12 aa, Artificial Sequence
  Synthetic polypeptide
IPGTILKLLKSL

SEQ ID NO: 54 - protein, 12 aa, Artificial Sequence
  Synthetic polypeptide
IQGTILKLLKSL

SEQ ID NO: 55 - protein, 12 aa, Artificial Sequence
  Synthetic polypeptide
IRGTILKLLKSL

SEQ ID NO: 56 - protein, 12 aa, Artificial Sequence
  Synthetic polypeptide
ISGTILKLLKSL

SEQ ID NO: 57 - protein, 12 aa, Artificial Sequence
  Synthetic polypeptide
ITGTILKLLKSL

SEQ ID NO: 58 - protein, 12 aa, Artificial Sequence
  Synthetic polypeptide
IVGTILKLLKSL

SEQ ID NO: 59 - protein, 12 aa, Artificial Sequence
  Synthetic polypeptide
IWGTILKLLKSL

SEQ ID NO: 60 - protein, 12 aa, Artificial Sequence
  Synthetic polypeptide
IYGTILKLLKSL

SEQ ID NO: 61 - protein, 12 aa, Artificial Sequence
  Synthetic polypeptide
ILATILKLLKSL

SEQ ID NO: 62 - protein, 12 aa, Artificial Sequence
  Synthetic polypeptide
ILCTILKLLKSL

SEQ ID NO: 63 - protein, 12 aa, Artificial Sequence
  Synthetic polypeptide
ILDTILKLLKSL

SEQ ID NO: 64 - protein, 12 aa, Artificial Sequence
  Synthetic polypeptide
ILETILKLLKSL

SEQ ID NO: 65 - protein, 12 aa, Artificial Sequence
  Synthetic polypeptide
ILFTILKLLKSL

SEQ ID NO: 66 - protein, 12 aa, Artificial Sequence
  Synthetic polypeptide
ILHTILKLLKSL

SEQ ID NO: 67 - protein, 12 aa, Artificial Sequence
  Synthetic polypeptide
ILITILKLLKSL

SEQ ID NO: 68 - protein, 12 aa, Artificial Sequence
  Synthetic polypeptide
ILKTILKLLKSL

SEQ ID NO: 69 - protein, 12 aa, Artificial Sequence
  Synthetic polypeptide
ILLTILKLLKSL

SEQ ID NO: 70 - protein, 12 aa, Artificial Sequence
  Synthetic polypeptide
ILMTILKLLKSL

SEQ ID NO: 71 - protein, 12 aa, Artificial Sequence
  Synthetic polypeptide
ILNTILKLLKSL

SEQ ID NO: 72 - protein, 12 aa, Artificial Sequence
  Synthetic polypeptide
ILPTILKLLKSL

SEQ ID NO: 73 - protein, 12 aa, Artificial Sequence
  Synthetic polypeptide
ILQTILKLLKSL

SEQ ID NO: 74 - protein, 12 aa, Artificial Sequence
  Synthetic polypeptide
ILRTILKLLKSL

SEQ ID NO: 75 - protein, 12 aa, Artificial Sequence
  Synthetic polypeptide
ILSTILKLLKSL

SEQ ID NO: 76 - protein, 12 aa, Artificial Sequence
  Synthetic polypeptide
ILTTILKLLKSL

SEQ ID NO: 77 - protein, 12 aa, Artificial Sequence
  Synthetic polypeptide
ILVTILKLLKSL

SEQ ID NO: 78 - protein, 12 aa, Artificial Sequence
  Synthetic polypeptide
ILWTILKLLKSL

SEQ ID NO: 79 - protein, 12 aa, Artificial Sequence
  Synthetic polypeptide
ILYTILKLLKSL

SEQ ID NO: 80 - protein, 12 aa, Artificial Sequence
  Synthetic polypeptide
ILGAILKLLKSL

SEQ ID NO: 81 - protein, 12 aa, Artificial Sequence
  Synthetic polypeptide
ILGCILKLLKSL

SEQ ID NO: 82 - protein, 12 aa, Artificial Sequence
  Synthetic polypeptide
ILGDILKLLKSL

SEQ ID NO: 83 - protein, 12 aa, Artificial Sequence
  Synthetic polypeptide
ILGEILKLLKSL

SEQ ID NO: 84 - protein, 12 aa, Artificial Sequence
  Synthetic polypeptide
ILGFILKLLKSL

SEQ ID NO: 85 - protein, 12 aa, Artificial Sequence
  Synthetic polypeptide
ILGGILKLLKSL

SEQ ID NO: 86 - protein, 12 aa, Artificial Sequence
  Synthetic polypeptide
ILGHILKLLKSL

SEQ ID NO: 87 - protein, 12 aa, Artificial Sequence
  Synthetic polypeptide
ILGIILKLLKSL

SEQ ID NO: 88 - protein, 12 aa, Artificial Sequence
  Synthetic polypeptide
ILGKILKLLKSL

SEQ ID NO: 89 - protein, 12 aa, Artificial Sequence
  Synthetic polypeptide
ILGLILKLLKSL

SEQ ID NO: 90 - protein, 12 aa, Artificial Sequence
  Synthetic polypeptide
ILGMILKLLKSL

SEQ ID NO: 91 - protein, 12 aa, Artificial Sequence
  Synthetic polypeptide
ILGNILKLLKSL

SEQ ID NO: 92 - protein, 12 aa, Artificial Sequence
  Synthetic polypeptide
ILGPILKLLKSL

SEQ ID NO: 93 - protein, 12 aa, Artificial Sequence
  Synthetic polypeptide
ILGQILKLLKSL

SEQ ID NO: 94 - protein, 12 aa, Artificial Sequence
  Synthetic polypeptide
ILGRILKLLKSL

SEQ ID NO: 95 - protein, 12 aa, Artificial Sequence
  Synthetic polypeptide
ILGSILKLLKSL

SEQ ID NO: 96 - protein, 12 aa, Artificial Sequence
  Synthetic polypeptide
ILGVILKLLKSL

SEQ ID NO: 97 - protein, 12 aa, Artificial Sequence
  Synthetic polypeptide
ILGWILKLLKSL

SEQ ID NO: 98 - protein, 12 aa, Artificial Sequence
  Synthetic polypeptide
ILGYILKLLKSL

SEQ ID NO: 99 - protein, 12 aa, Artificial Sequence
  Synthetic polypeptide
ILGTIAKLLKSL

SEQ ID NO: 100 - protein, 12 aa, Artificial Sequence
  Synthetic polypeptide
ILGTICKLLKSL

SEQ ID NO: 101 - protein, 12 aa, Artificial Sequence
  Synthetic polypeptide
ILGTIDKLLKSL

SEQ ID NO: 102 - protein, 12 aa, Artificial Sequence
  Synthetic polypeptide
ILGTIEKLLKSL

SEQ ID NO: 103 - protein, 12 aa, Artificial Sequence
  Synthetic polypeptide
ILGTIFKLLKSL

SEQ ID NO: 104 - protein, 12 aa, Artificial Sequence
  Synthetic polypeptide
ILGTIGKLLKSL

SEQ ID NO: 105 - protein, 12 aa, Artificial Sequence
  Synthetic polypeptide
ILGTIHKLLKSL

SEQ ID NO: 106 - protein, 12 aa, Artificial Sequence
  Synthetic polypeptide
ILGTIIKLLKSL

SEQ ID NO: 107 - protein, 12 aa, Artificial Sequence
  Synthetic polypeptide
ILGTIKKLLKSL

SEQ ID NO: 108 - protein, 12 aa, Artificial Sequence
  Synthetic polypeptide
ILGTIMKLLKSL

SEQ ID NO: 109 - protein, 12 aa, Artificial Sequence
  Synthetic polypeptide
ILGTINKLLKSL

SEQ ID NO: 110 - protein, 12 aa, Artificial Sequence
  Synthetic polypeptide
ILGTIPKLLKSL

SEQ ID NO: 111 - protein, 12 aa, Artificial Sequence
  Synthetic polypeptide
ILGTIQKLLKSL

SEQ ID NO: 112 - protein, 12 aa, Artificial Sequence
  Synthetic polypeptide
ILGTIRKLLKSL

SEQ ID NO: 113 - protein, 12 aa, Artificial Sequence
  Synthetic polypeptide
ILGTISKLLKSL

SEQ ID NO: 114 - protein, 12 aa, Artificial Sequence
  Synthetic polypeptide
ILGTITKLLKSL

SEQ ID NO: 115 - protein, 12 aa, Artificial Sequence
  Synthetic polypeptide
ILGTIVKLLKSL

SEQ ID NO: 116 - protein, 12 aa, Artificial Sequence
  Synthetic polypeptide
ILGTIWKLLKSL

SEQ ID NO: 117 - protein, 12 aa, Artificial Sequence
  Synthetic polypeptide
ILGTIYKLLKSL

SEQ ID NO: 118 - protein, 12 aa, Artificial Sequence
  Synthetic polypeptide
ILGTILKALKSL

SEQ ID NO: 119 - protein, 12 aa, Artificial Sequence
  Synthetic polypeptide
ILGTILKCLKSL

SEQ ID NO: 120 - protein, 12 aa, Artificial Sequence
  Synthetic polypeptide
ILGTILKDLKSL

SEQ ID NO: 121 - protein, 12 aa, Artificial Sequence
  Synthetic polypeptide
ILGTILKELKSL

SEQ ID NO: 122 - protein, 12 aa, Artificial Sequence
  Synthetic polypeptide
ILGTILKFLKSL

SEQ ID NO: 123 - protein, 12 aa, Artificial Sequence
  Synthetic polypeptide
ILGTILKGLKSL

SEQ ID NO: 124 - protein, 12 aa, Artificial Sequence
  Synthetic polypeptide
ILGTILKHLKSL

SEQ ID NO: 125 - protein, 12 aa, Artificial Sequence
  Synthetic polypeptide
ILGTILKILKSL

SEQ ID NO: 126 - protein, 12 aa, Artificial Sequence
  Synthetic polypeptide
ILGTILKKLKSL

SEQ ID NO: 127 - protein, 12 aa, Artificial Sequence
  Synthetic polypeptide
ILGTILKMLKSL

SEQ ID NO: 128 - protein, 12 aa, Artificial Sequence
  Synthetic polypeptide
ILGTILKNLKSL

SEQ ID NO: 129 - protein, 12 aa, Artificial Sequence
  Synthetic polypeptide
ILGTILKPLKSL

SEQ ID NO: 130 - protein, 12 aa, Artificial Sequence
  Synthetic polypeptide
ILGTILKQLKSL

SEQ ID NO: 131 - protein, 12 aa, Artificial Sequence
  Synthetic polypeptide
ILGTILKRLKSL

SEQ ID NO: 132 - protein, 12 aa, Artificial Sequence
  Synthetic polypeptide
ILGTILKSLKSL

SEQ ID NO: 133 - protein, 12 aa, Artificial Sequence
  Synthetic polypeptide
ILGTILKTLKSL

SEQ ID NO: 134 - protein, 12 aa, Artificial Sequence
  Synthetic polypeptide
ILGTILKVLKSL

SEQ ID NO: 135 - protein, 12 aa, Artificial Sequence
  Synthetic polypeptide
ILGTILKWLKSL

SEQ ID NO: 136 - protein, 12 aa, Artificial Sequence
  Synthetic polypeptide
ILGTILKYLKSL

SEQ ID NO: 137 - protein, 12 aa, Artificial Sequence
  Synthetic polypeptide
ILGTILKLAKSL

SEQ ID NO: 138 - protein, 12 aa, Artificial Sequence
  Synthetic polypeptide
ILGTILKLCKSL

SEQ ID NO: 139 - protein, 12 aa, Artificial Sequence
  Synthetic polypeptide
ILGTILKLDKSL

SEQ ID NO: 140 - protein, 12 aa, Artificial Sequence
  Synthetic polypeptide
ILGTILKLEKSL

SEQ ID NO: 141 - protein, 12 aa, Artificial Sequence
  Synthetic polypeptide
ILGTILKLFKSL

SEQ ID NO: 142 - protein, 12 aa, Artificial Sequence
  Synthetic polypeptide
ILGTILKLGKSL

SEQ ID NO: 143 - protein, 12 aa, Artificial Sequence
  Synthetic polypeptide
ILGTILKLHKSL

SEQ ID NO: 144 - protein, 12 aa, Artificial Sequence
  Synthetic polypeptide
ILGTILKLIKSL

SEQ ID NO: 145 - protein, 12 aa, Artificial Sequence
  Synthetic polypeptide
ILGTILKLKKSL

SEQ ID NO: 146 - protein, 12 aa, Artificial Sequence
  Synthetic polypeptide
ILGTILKLMKSL

SEQ ID NO: 147 - protein, 12 aa, Artificial Sequence
  Synthetic polypeptide
ILGTILKLNKSL

SEQ ID NO: 148 - protein, 12 aa, Artificial Sequence
  Synthetic polypeptide
ILGTILKLPKSL

SEQ ID NO: 149 - protein, 12 aa, Artificial Sequence
  Synthetic polypeptide
ILGTILKLQKSL

SEQ ID NO: 150 - protein, 12 aa, Artificial Sequence
  Synthetic polypeptide
ILGTILKLRKSL

SEQ ID NO: 151 - protein, 12 aa, Artificial Sequence
  Synthetic polypeptide
ILGTILKLSKSL

SEQ ID NO: 152 - protein, 12 aa, Artificial Sequence
  Synthetic polypeptide
ILGTILKLTKSL

SEQ ID NO: 153 - protein, 12 aa, Artificial Sequence
  Synthetic polypeptide
ILGTILKLVKSL

SEQ ID NO: 154 - protein, 12 aa, Artificial Sequence
  Synthetic polypeptide
ILGTILKLWKSL

SEQ ID NO: 155 - protein, 12 aa, Artificial Sequence
  Synthetic polypeptide
ILGTILKLYKSL

SEQ ID NO: 156 - protein, 12 aa, Artificial Sequence
  Synthetic polypeptide
ILGTILKLLASL

SEQ ID NO: 157 - protein, 12 aa, Artificial Sequence
  Synthetic polypeptide
ILGTILKLLCSL

SEQ ID NO: 158 - protein, 12 aa, Artificial Sequence
  Synthetic polypeptide
ILGTILKLLDSL

SEQ ID NO: 159 - protein, 12 aa, Artificial Sequence
  Synthetic polypeptide
ILGTILKLLESL

SEQ ID NO: 160 - protein, 12 aa, Artificial Sequence
  Synthetic polypeptide
ILGTILKLLFSL

SEQ ID NO: 161 - protein, 12 aa, Artificial Sequence
  Synthetic polypeptide
ILGTILKLLGSL

SEQ ID NO: 162 - protein, 12 aa, Artificial Sequence
  Synthetic polypeptide
ILGTILKLLHSL

SEQ ID NO: 163 - protein, 12 aa, Artificial Sequence
  Synthetic polypeptide
ILGTILKLLISL

SEQ ID NO: 164 - protein, 12 aa, Artificial Sequence
  Synthetic polypeptide
ILGTILKLLLSL

SEQ ID NO: 165 - protein, 12 aa, Artificial Sequence
  Synthetic polypeptide
ILGTILKLLMSL

SEQ ID NO: 166 - protein, 12 aa, Artificial Sequence
  Synthetic polypeptide
ILGTILKLLNSL

SEQ ID NO: 167 - protein, 12 aa, Artificial Sequence
  Synthetic polypeptide
ILGTILKLLPSL

SEQ ID NO: 168 - protein, 12 aa, Artificial Sequence
  Synthetic polypeptide
ILGTILKLLQSL

SEQ ID NO: 169 - protein, 12 aa, Artificial Sequence
  Synthetic polypeptide
ILGTILKLLRSL

SEQ ID NO: 170 - protein, 12 aa, Artificial Sequence
  Synthetic polypeptide
ILGTILKLLSSL

SEQ ID NO: 171 - protein, 12 aa, Artificial Sequence
  Synthetic polypeptide
ILGTILKLLTSL

SEQ ID NO: 172 - protein, 12 aa, Artificial Sequence
  Synthetic polypeptide
ILGTILKLLVSL

SEQ ID NO: 173 - protein, 12 aa, Artificial Sequence
  Synthetic polypeptide
ILGTILKLLWSL

SEQ ID NO: 174 - protein, 12 aa, Artificial Sequence
  Synthetic polypeptide
ILGTILKLLYSL

SEQ ID NO: 175 - protein, 12 aa, Artificial Sequence
  Synthetic polypeptide
ILGTILKLLKAL

SEQ ID NO: 176 - protein, 12 aa, Artificial Sequence
  Synthetic polypeptide
ILGTILKLLKCL

SEQ ID NO: 177 - protein, 12 aa, Artificial Sequence
  Synthetic polypeptide
ILGTILKLLKDL

SEQ ID NO: 178 - protein, 12 aa, Artificial Sequence
  Synthetic polypeptide
ILGTILKLLKEL

SEQ ID NO: 179 - protein, 12 aa, Artificial Sequence
  Synthetic polypeptide
ILGTILKLLKFL

SEQ ID NO: 180 - protein, 12 aa, Artificial Sequence
  Synthetic polypeptide
ILGTILKLLKGL

SEQ ID NO: 181 - protein, 12 aa, Artificial Sequence
  Synthetic polypeptide
ILGTILKLLKHL

SEQ ID NO: 182 - protein, 12 aa, Artificial Sequence
  Synthetic polypeptide
ILGTILKLLKIL

SEQ ID NO: 183 - protein, 12 aa, Artificial Sequence
  Synthetic polypeptide
ILGTILKLLKKL

SEQ ID NO: 184 - protein, 12 aa, Artificial Sequence
  Synthetic polypeptide
ILGTILKLLKLL

SEQ ID NO: 185 - protein, 12 aa, Artificial Sequence
  Synthetic polypeptide
ILGTILKLLKML

SEQ ID NO: 186 - protein, 12 aa, Artificial Sequence
  Synthetic polypeptide
ILGTILKLLKNL

SEQ ID NO: 187 - protein, 12 aa, Artificial Sequence
  Synthetic polypeptide
ILGTILKLLKPL

SEQ ID NO: 188 - protein, 12 aa, Artificial Sequence
  Synthetic polypeptide
ILGTILKLLKQL

SEQ ID NO: 189 - protein, 12 aa, Artificial Sequence
  Synthetic polypeptide
ILGTILKLLKRL

SEQ ID NO: 190 - protein, 12 aa, Artificial Sequence
  Synthetic polypeptide
ILGTILKLLKTL

SEQ ID NO: 191 - protein, 12 aa, Artificial Sequence
  Synthetic polypeptide
ILGTILKLLKVL

SEQ ID NO: 192 - protein, 12 aa, Artificial Sequence
  Synthetic polypeptide
ILGTILKLLKWL

SEQ ID NO: 193 - protein, 12 aa, Artificial Sequence
  Synthetic polypeptide
ILGTILKLLKYL

SEQ ID NO: 194 - protein, 12 aa, Artificial Sequence
  Synthetic polypeptide
ILGTILKLLKSA

SEQ ID NO: 195 - protein, 12 aa, Artificial Sequence
  Synthetic polypeptide
ILGTILKLLKSC

SEQ ID NO: 196 - protein, 12 aa, Artificial Sequence
  Synthetic polypeptide
ILGTILKLLKSD

SEQ ID NO: 197 - protein, 12 aa, Artificial Sequence
  Synthetic polypeptide
ILGTILKLLKSE

SEQ ID NO: 198 - protein, 12 aa, Artificial Sequence
  Synthetic polypeptide
ILGTILKLLKSF

SEQ ID NO: 199 - protein, 12 aa, Artificial Sequence
  Synthetic polypeptide
ILGTILKLLKSG

SEQ ID NO: 200 - protein, 12 aa, Artificial Sequence
  Synthetic polypeptide
ILGTILKLLKSH

SEQ ID NO: 201 - protein, 12 aa, Artificial Sequence
  Synthetic polypeptide
ILGTILKLLKSI

SEQ ID NO: 202 - protein, 12 aa, Artificial Sequence
  Synthetic polypeptide
ILGTILKLLKSK

SEQ ID NO: 203 - protein, 12 aa, Artificial Sequence
  Synthetic polypeptide
ILGTILKLLKSM

SEQ ID NO: 204 - protein, 12 aa, Artificial Sequence
  Synthetic polypeptide
ILGTILKLLKSN

SEQ ID NO: 205 - protein, 12 aa, Artificial Sequence
  Synthetic polypeptide
ILGTILKLLKSP

SEQ ID NO: 206 - protein, 12 aa, Artificial Sequence
  Synthetic polypeptide
ILGTILKLLKSQ

SEQ ID NO: 207 - protein, 12 aa, Artificial Sequence
  Synthetic polypeptide
ILGTILKLLKSR

SEQ ID NO: 208 - protein, 12 aa, Artificial Sequence
  Synthetic polypeptide
ILGTILKLLKSS

SEQ ID NO: 209 - protein, 12 aa, Artificial Sequence
  Synthetic polypeptide
ILGTILKLLKST

SEQ ID NO: 210 - protein, 12 aa, Artificial Sequence
  Synthetic polypeptide
ILGTILKLLKSV

SEQ ID NO: 211 - protein, 12 aa, Artificial Sequence
  Synthetic polypeptide
ILGTILKLLKSW

SEQ ID NO: 212 - protein, 12 aa, Artificial Sequence
  Synthetic polypeptide
ILGTILKLLKSY

SEQ ID NO: 213 - protein, 11 aa, Artificial Sequence
  Synthetic polypeptide
ALLSLIRKLLT

SEQ ID NO: 214 - protein, 11 aa, Artificial Sequence
  Synthetic polypeptide
CLLSLIRKLLT

SEQ ID NO: 215 - protein, 11 aa, Artificial Sequence
  Synthetic polypeptide
DLLSLIRKLLT

SEQ ID NO: 216 - protein, 11 aa, Artificial Sequence
  Synthetic polypeptide
ELLSLIRKLLT

SEQ ID NO: 217 - protein, 11 aa, Artificial Sequence
  Synthetic polypeptide
FLLSLIRKLLT

SEQ ID NO: 218 - protein, 11 aa, Artificial Sequence
  Synthetic polypeptide
GLLSLIRKLLT

SEQ ID NO: 219 - protein, 11 aa, Artificial Sequence
  Synthetic polypeptide
HLLSLIRKLLT

SEQ ID NO: 220 - protein, 11 aa, Artificial Sequence
  Synthetic polypeptide
ILLSLIRKLLT

SEQ ID NO: 221 - protein, 11 aa, Artificial Sequence
  Synthetic polypeptide
KLLSLIRKLLT

SEQ ID NO: 222 - protein, 11 aa, Artificial Sequence
  Synthetic polypeptide
LLLSLIRKLLT

SEQ ID NO: 223 - protein, 11 aa, Artificial Sequence
  Synthetic polypeptide
MLLSLIRKLLT

SEQ ID NO: 224 - protein, 11 aa, Artificial Sequence
  Synthetic polypeptide
NLLSLIRKLLT

SEQ ID NO: 225 - protein, 11 aa, Artificial Sequence
  Synthetic polypeptide
PLLSLIRKLLT

SEQ ID NO: 226 - protein, 11 aa, Artificial Sequence
  Synthetic polypeptide
QLLSLIRKLLT

SEQ ID NO: 227 - protein, 11 aa, Artificial Sequence
  Synthetic polypeptide
RLLSLIRKLLT

SEQ ID NO: 228 - protein, 11 aa, Artificial Sequence
  Synthetic polypeptide
TLLSLIRKLLT

SEQ ID NO: 229 - protein, 11 aa, Artificial Sequence
  Synthetic polypeptide
VLLSLIRKLLT

SEQ ID NO: 230 - protein, 11 aa, Artificial Sequence
  Synthetic polypeptide
WLLSLIRKLLT

SEQ ID NO: 231 - protein, 11 aa, Artificial Sequence
  Synthetic polypeptide
YLLSLIRKLLT

SEQ ID NO: 232 - protein, 11 aa, Artificial Sequence
  Synthetic polypeptide
SALSLIRKLLT

SEQ ID NO: 233 - protein, 11 aa, Artificial Sequence
  Synthetic polypeptide
SCLSLIRKLLT

SEQ ID NO: 234 - protein, 11 aa, Artificial Sequence
  Synthetic polypeptide
SDLSLIRKLLT

SEQ ID NO: 235 - protein, 11 aa, Artificial Sequence
  Synthetic polypeptide
SELSLIRKLLT

SEQ ID NO: 236 - protein, 11 aa, Artificial Sequence
  Synthetic polypeptide
SFLSLIRKLLT

SEQ ID NO: 237 - protein, 11 aa, Artificial Sequence
  Synthetic polypeptide
SGLSLIRKLLT

SEQ ID NO: 238 - protein, 11 aa, Artificial Sequence
  Synthetic polypeptide
SHLSLIRKLLT

SEQ ID NO: 239 - protein, 11 aa, Artificial Sequence
  Synthetic polypeptide
SILSLIRKLLT

SEQ ID NO: 240 - protein, 11 aa, Artificial Sequence
  Synthetic polypeptide
SKLSLIRKLLT

SEQ ID NO: 241 - protein, 11 aa, Artificial Sequence
  Synthetic polypeptide
SMLSLIRKLLT

SEQ ID NO: 242 - protein, 11 aa, Artificial Sequence
  Synthetic polypeptide
SNLSLIRKLLT

SEQ ID NO: 243 - protein, 11 aa, Artificial Sequence
  Synthetic polypeptide
SPLSLIRKLLT

SEQ ID NO: 244 - protein, 11 aa, Artificial Sequence
  Synthetic polypeptide
SQLSLIRKLLT

SEQ ID NO: 245 - protein, 11 aa, Artificial Sequence
  Synthetic polypeptide
SRLSLIRKLLT

SEQ ID NO: 246 - protein, 11 aa, Artificial Sequence
  Synthetic polypeptide
SSLSLIRKLLT

SEQ ID NO: 247 - protein, 11 aa, Artificial Sequence
  Synthetic polypeptide
STLSLIRKLLT

SEQ ID NO: 248 - protein, 11 aa, Artificial Sequence
  Synthetic polypeptide
SVLSLIRKLLT

SEQ ID NO: 249 - protein, 11 aa, Artificial Sequence
  Synthetic polypeptide
SWLSLIRKLLT

SEQ ID NO: 250 - protein, 11 aa, Artificial Sequence
  Synthetic polypeptide
SYLSLIRKLLT

SEQ ID NO: 251 - protein, 11 aa, Artificial Sequence
  Synthetic polypeptide
SLASLIRKLLT

SEQ ID NO: 252 - protein, 11 aa, Artificial Sequence
  Synthetic polypeptide
SLCSLIRKLLT

SEQ ID NO: 253 - protein, 11 aa, Artificial Sequence
  Synthetic polypeptide
SLDSLIRKLLT

SEQ ID NO: 254 - protein, 11 aa, Artificial Sequence
  Synthetic polypeptide
SLESLIRKLLT

SEQ ID NO: 255 - protein, 11 aa, Artificial Sequence
  Synthetic polypeptide
SLFSLIRKLLT

SEQ ID NO: 256 - protein, 11 aa, Artificial Sequence
  Synthetic polypeptide
SLGSLIRKLLT

SEQ ID NO: 257 - protein, 11 aa, Artificial Sequence
  Synthetic polypeptide
SLHSLIRKLLT

SEQ ID NO: 258 - protein, 11 aa, Artificial Sequence
  Synthetic polypeptide
SLISLIRKLLT

SEQ ID NO: 259 - protein, 11 aa, Artificial Sequence
  Synthetic polypeptide
SLKSLIRKLLT

SEQ ID NO: 260 - protein, 11 aa, Artificial Sequence
  Synthetic polypeptide
SLMSLIRKLLT

SEQ ID NO: 261 - protein, 11 aa, Artificial Sequence
  Synthetic polypeptide
SLNSLIRKLLT

SEQ ID NO: 262 - protein, 11 aa, Artificial Sequence
  Synthetic polypeptide
SLPSLIRKLLT

SEQ ID NO: 263 - protein, 11 aa, Artificial Sequence
  Synthetic polypeptide
SLQSLIRKLLT

SEQ ID NO: 264 - protein, 11 aa, Artificial Sequence
  Synthetic polypeptide
SLRSLIRKLLT

SEQ ID NO: 265 - protein, 11 aa, Artificial Sequence
  Synthetic polypeptide
SLSSLIRKLLT

SEQ ID NO: 266 - protein, 11 aa, Artificial Sequence
  Synthetic polypeptide
SLTSLIRKLLT

SEQ ID NO: 267 - protein, 11 aa, Artificial Sequence
  Synthetic polypeptide
SLVSLIRKLLT

SEQ ID NO: 268 - protein, 11 aa, Artificial Sequence
  Synthetic polypeptide
SLWSLIRKLLT

SEQ ID NO: 269 - protein, 11 aa, Artificial Sequence
  Synthetic polypeptide
SLYSLIRKLLT

SEQ ID NO: 270 - protein, 11 aa, Artificial Sequence
  Synthetic polypeptide
SLLALIRKLLT

SEQ ID NO: 271 - protein, 11 aa, Artificial Sequence
  Synthetic polypeptide
SLLCLIRKLLT

SEQ ID NO: 272 - protein, 11 aa, Artificial Sequence
  Synthetic polypeptide
SLLDLIRKLLT

SEQ ID NO: 273 - protein, 11 aa, Artificial Sequence
  Synthetic polypeptide
SLLELIRKLLT

SEQ ID NO: 274 - protein, 11 aa, Artificial Sequence
  Synthetic polypeptide
SLLFLIRKLLT

SEQ ID NO: 275 - protein, 11 aa, Artificial Sequence
  Synthetic polypeptide
SLLGLIRKLLT

SEQ ID NO: 276 - protein, 11 aa, Artificial Sequence
  Synthetic polypeptide
SLLHLIRKLLT

SEQ ID NO: 277 - protein, 11 aa, Artificial Sequence
  Synthetic polypeptide
SLLILIRKLLT

SEQ ID NO: 278 - protein, 11 aa, Artificial Sequence
  Synthetic polypeptide
SLLKLIRKLLT

SEQ ID NO: 279 - protein, 11 aa, Artificial Sequence
  Synthetic polypeptide
SLLLLIRKLLT

SEQ ID NO: 280 - protein, 11 aa, Artificial Sequence
  Synthetic polypeptide
SLLMLIRKLLT

SEQ ID NO: 281 - protein, 11 aa, Artificial Sequence
  Synthetic polypeptide
SLLNLIRKLLT

SEQ ID NO: 282 - protein, 11 aa, Artificial Sequence
  Synthetic polypeptide
SLLPLIRKLLT

SEQ ID NO: 283 - protein, 11 aa, Artificial Sequence
  Synthetic polypeptide
SLLQLIRKLLT

SEQ ID NO: 284 - protein, 11 aa, Artificial Sequence
  Synthetic polypeptide
SLLRLIRKLLT

SEQ ID NO: 285 - protein, 11 aa, Artificial Sequence
  Synthetic polypeptide
SLLTLIRKLLT

SEQ ID NO: 286 - protein, 11 aa, Artificial Sequence
  Synthetic polypeptide
SLLVLIRKLLT

SEQ ID NO: 287 - protein, 11 aa, Artificial Sequence
  Synthetic polypeptide
SLLWLIRKLLT

SEQ ID NO: 288 - protein, 11 aa, Artificial Sequence
  Synthetic polypeptide
SLLYLIRKLLT

SEQ ID NO: 289 - protein, 11 aa, Artificial Sequence
  Synthetic polypeptide
SLLSAIRKLLT

SEQ ID NO: 290 - protein, 11 aa, Artificial Sequence
  Synthetic polypeptide
SLLSCIRKLLT

SEQ ID NO: 291 - protein, 11 aa, Artificial Sequence
  Synthetic polypeptide
SLLSDIRKLLT

SEQ ID NO: 292 - protein, 11 aa, Artificial Sequence
  Synthetic polypeptide
SLLSEIRKLLT

SEQ ID NO: 293 - protein, 11 aa, Artificial Sequence
  Synthetic polypeptide
SLLSFIRKLLT

SEQ ID NO: 294 - protein, 11 aa, Artificial Sequence
  Synthetic polypeptide
SLLSGIRKLLT

SEQ ID NO: 295 - protein, 11 aa, Artificial Sequence
  Synthetic polypeptide
SLLSHIRKLLT

SEQ ID NO: 296 - protein, 11 aa, Artificial Sequence
  Synthetic polypeptide
SLLSIIRKLLT

SEQ ID NO: 297 - protein, 11 aa, Artificial Sequence
  Synthetic polypeptide
SLLSKIRKLLT

SEQ ID NO: 298 - protein, 11 aa, Artificial Sequence
  Synthetic polypeptide
SLLSMIRKLLT

SEQ ID NO: 299 - protein, 11 aa, Artificial Sequence
  Synthetic polypeptide
SLLSNIRKLLT

SEQ ID NO: 300 - protein, 11 aa, Artificial Sequence
  Synthetic polypeptide
SLLSPIRKLLT

SEQ ID NO: 301 - protein, 11 aa, Artificial Sequence
  Synthetic polypeptide
SLLSQIRKLLT

SEQ ID NO: 302 - protein, 11 aa, Artificial Sequence
  Synthetic polypeptide
SLLSRIRKLLT

SEQ ID NO: 303 - protein, 11 aa, Artificial Sequence
  Synthetic polypeptide
SLLSSIRKLLT

SEQ ID NO: 304 - protein, 11 aa, Artificial Sequence
  Synthetic polypeptide
SLLSTIRKLLT

SEQ ID NO: 305 - protein, 11 aa, Artificial Sequence
  Synthetic polypeptide
SLLSVIRKLLT

SEQ ID NO: 306 - protein, 11 aa, Artificial Sequence
  Synthetic polypeptide
SLLSWIRKLLT

SEQ ID NO: 307 - protein, 11 aa, Artificial Sequence
  Synthetic polypeptide
SLLSYIRKLLT

SEQ ID NO: 308 - protein, 11 aa, Artificial Sequence
  Synthetic polypeptide
SLLSLARKLLT

SEQ ID NO: 309 - protein, 11 aa, Artificial Sequence
  Synthetic polypeptide
SLLSLCRKLLT

SEQ ID NO: 310 - protein, 11 aa, Artificial Sequence
  Synthetic polypeptide
SLLSLDRKLLT

SEQ ID NO: 311 - protein, 11 aa, Artificial Sequence
  Synthetic polypeptide
SLLSLERKLLT

SEQ ID NO: 312 - protein, 11 aa, Artificial Sequence
  Synthetic polypeptide
SLLSLFRKLLT

SEQ ID NO: 313 - protein, 11 aa, Artificial Sequence
  Synthetic polypeptide
SLLSLGRKLLT

SEQ ID NO: 314 - protein, 11 aa, Artificial Sequence
  Synthetic polypeptide
SLLSLHRKLLT

SEQ ID NO: 315 - protein, 11 aa, Artificial Sequence
  Synthetic polypeptide
SLLSLKRKLLT

SEQ ID NO: 316 - protein, 11 aa, Artificial Sequence
  Synthetic polypeptide
SLLSLLRKLLT

SEQ ID NO: 317 - protein, 11 aa, Artificial Sequence
  Synthetic polypeptide
SLLSLMRKLLT

SEQ ID NO: 318 - protein, 11 aa, Artificial Sequence
  Synthetic polypeptide
SLLSLNRKLLT

SEQ ID NO: 319 - protein, 11 aa, Artificial Sequence
  Synthetic polypeptide
SLLSLPRKLLT

SEQ ID NO: 320 - protein, 11 aa, Artificial Sequence
  Synthetic polypeptide
SLLSLQRKLLT

SEQ ID NO: 321 - protein, 11 aa, Artificial Sequence
  Synthetic polypeptide
SLLSLRRKLLT

SEQ ID NO: 322 - protein, 11 aa, Artificial Sequence
  Synthetic polypeptide
SLLSLSRKLLT

SEQ ID NO: 323 - protein, 11 aa, Artificial Sequence
  Synthetic polypeptide
SLLSLTRKLLT

SEQ ID NO: 324 - protein, 11 aa, Artificial Sequence
  Synthetic polypeptide
SLLSLVRKLLT

SEQ ID NO: 325 - protein, 11 aa, Artificial Sequence
  Synthetic polypeptide
SLLSLWRKLLT

SEQ ID NO: 326 - protein, 11 aa, Artificial Sequence
  Synthetic polypeptide
SLLSLYRKLLT

SEQ ID NO: 327 - protein, 11 aa, Artificial Sequence
  Synthetic polypeptide
SLLSLIAKLLT

SEQ ID NO: 328 - protein, 11 aa, Artificial Sequence
  Synthetic polypeptide
SLLSLICKLLT

SEQ ID NO: 329 - protein, 11 aa, Artificial Sequence
  Synthetic polypeptide
SLLSLIDKLLT

SEQ ID NO: 330 - protein, 11 aa, Artificial Sequence
  Synthetic polypeptide
SLLSLIEKLLT

SEQ ID NO: 331 - protein, 11 aa, Artificial Sequence
  Synthetic polypeptide
SLLSLIFKLLT

SEQ ID NO: 332 - protein, 11 aa, Artificial Sequence
  Synthetic polypeptide
SLLSLIGKLLT

SEQ ID NO: 333 - protein, 11 aa, Artificial Sequence
  Synthetic polypeptide
SLLSLIHKLLT

SEQ ID NO: 334 - protein, 11 aa, Artificial Sequence
  Synthetic polypeptide
SLLSLIIKLLT

SEQ ID NO: 335 - protein, 11 aa, Artificial Sequence
  Synthetic polypeptide
SLLSLIKKLLT

SEQ ID NO: 336 - protein, 11 aa, Artificial Sequence
  Synthetic polypeptide
SLLSLILKLLT

SEQ ID NO: 337 - protein, 11 aa, Artificial Sequence
  Synthetic polypeptide
SLLSLIMKLLT

SEQ ID NO: 338 - protein, 11 aa, Artificial Sequence
  Synthetic polypeptide
SLLSLINKLLT

SEQ ID NO: 339 - protein, 11 aa, Artificial Sequence
  Synthetic polypeptide
SLLSLIPKLLT

SEQ ID NO: 340 - protein, 11 aa, Artificial Sequence
  Synthetic polypeptide
SLLSLIQKLLT

SEQ ID NO: 341 - protein, 11 aa, Artificial Sequence
  Synthetic polypeptide
SLLSLISKLLT

SEQ ID NO: 342 - protein, 11 aa, Artificial Sequence
  Synthetic polypeptide
SLLSLITKLLT

SEQ ID NO: 343 - protein, 11 aa, Artificial Sequence
  Synthetic polypeptide
SLLSLIVKLLT

SEQ ID NO: 344 - protein, 11 aa, Artificial Sequence
  Synthetic polypeptide
SLLSLIWKLLT

SEQ ID NO: 345 - protein, 11 aa, Artificial Sequence
  Synthetic polypeptide
SLLSLIYKLLT

SEQ ID NO: 346 - protein, 11 aa, Artificial Sequence
  Synthetic polypeptide
SLLSLIRKALT

SEQ ID NO: 347 - protein, 11 aa, Artificial Sequence
  Synthetic polypeptide
SLLSLIRKCLT

SEQ ID NO: 348 - protein, 11 aa, Artificial Sequence
  Synthetic polypeptide
SLLSLIRKDLT

SEQ ID NO: 349 - protein, 11 aa, Artificial Sequence
  Synthetic polypeptide
SLLSLIRKELT

SEQ ID NO: 350 - protein, 11 aa, Artificial Sequence
  Synthetic polypeptide
SLLSLIRKFLT

SEQ ID NO: 351 - protein, 11 aa, Artificial Sequence
  Synthetic polypeptide
SLLSLIRKGLT

SEQ ID NO: 352 - protein, 11 aa, Artificial Sequence
  Synthetic polypeptide
SLLSLIRKHLT

SEQ ID NO: 353 - protein, 11 aa, Artificial Sequence
  Synthetic polypeptide
SLLSLIRKILT

SEQ ID NO: 354 - protein, 11 aa, Artificial Sequence
  Synthetic polypeptide
SLLSLIRKKLT

SEQ ID NO: 355 - protein, 11 aa, Artificial Sequence
  Synthetic polypeptide
SLLSLIRKMLT

SEQ ID NO: 356 - protein, 11 aa, Artificial Sequence
  Synthetic polypeptide
SLLSLIRKNLT

SEQ ID NO: 357 - protein, 11 aa, Artificial Sequence
  Synthetic polypeptide
SLLSLIRKPLT

SEQ ID NO: 358 - protein, 11 aa, Artificial Sequence
  Synthetic polypeptide
SLLSLIRKQLT

SEQ ID NO: 359 - protein, 11 aa, Artificial Sequence
  Synthetic polypeptide
SLLSLIRKRLT

SEQ ID NO: 360 - protein, 11 aa, Artificial Sequence
  Synthetic polypeptide
SLLSLIRKSLT

SEQ ID NO: 361 - protein, 11 aa, Artificial Sequence
  Synthetic polypeptide
SLLSLIRKTLT

SEQ ID NO: 362 - protein, 11 aa, Artificial Sequence
  Synthetic polypeptide
SLLSLIRKVLT

SEQ ID NO: 363 - protein, 11 aa, Artificial Sequence
  Synthetic polypeptide
SLLSLIRKWLT

SEQ ID NO: 364 - protein, 11 aa, Artificial Sequence
  Synthetic polypeptide
SLLSLIRKYLT

SEQ ID NO: 365 - protein, 11 aa, Artificial Sequence
  Synthetic polypeptide
SLLSLIRKLLA

SEQ ID NO: 366 - protein, 11 aa, Artificial Sequence
  Synthetic polypeptide
SLLSLIRKLLC

SEQ ID NO: 367 - protein, 11 aa, Artificial Sequence
  Synthetic polypeptide
SLLSLIRKLLD

SEQ ID NO: 368 - protein, 11 aa, Artificial Sequence
  Synthetic polypeptide
SLLSLIRKLLE

SEQ ID NO: 369 - protein, 11 aa, Artificial Sequence
  Synthetic polypeptide
SLLSLIRKLLF

SEQ ID NO: 370 - protein, 11 aa, Artificial Sequence
  Synthetic polypeptide
SLLSLIRKLLG

SEQ ID NO: 371 - protein, 11 aa, Artificial Sequence
  Synthetic polypeptide
SLLSLIRKLLH

SEQ ID NO: 372 - protein, 11 aa, Artificial Sequence
  Synthetic polypeptide
SLLSLIRKLLI

SEQ ID NO: 373 - protein, 11 aa, Artificial Sequence
  Synthetic polypeptide
SLLSLIRKLLK

SEQ ID NO: 374 - protein, 11 aa, Artificial Sequence
  Synthetic polypeptide
SLLSLIRKLLL

SEQ ID NO: 375 - protein, 11 aa, Artificial Sequence
  Synthetic polypeptide
SLLSLIRKLLM

SEQ ID NO: 376 - protein, 11 aa, Artificial Sequence
  Synthetic polypeptide
SLLSLIRKLLN

SEQ ID NO: 377 - protein, 11 aa, Artificial Sequence
  Synthetic polypeptide
SLLSLIRKLLP

SEQ ID NO: 378 - protein, 11 aa, Artificial Sequence
  Synthetic polypeptide
SLLSLIRKLLQ

SEQ ID NO: 379 - protein, 11 aa, Artificial Sequence
  Synthetic polypeptide
SLLSLIRKLLR

SEQ ID NO: 380 - protein, 11 aa, Artificial Sequence
  Synthetic polypeptide
SLLSLIRKLLS

SEQ ID NO: 381 - protein, 11 aa, Artificial Sequence
  Synthetic polypeptide
SLLSLIRKLLV

SEQ ID NO: 382 - protein, 11 aa, Artificial Sequence
  Synthetic polypeptide
SLLSLIRKLLW

SEQ ID NO: 383 - protein, 11 aa, Artificial Sequence
  Synthetic polypeptide
SLLSLIRKLLY

SEQ ID NO: 384 - protein, 14 aa, Artificial Sequence
  Synthetic polypeptide
CILGTILKLLKSLC

SEQ ID NO: 385 - protein, 13 aa, Artificial Sequence
  Synthetic polypeptide
CSLLSLIRKLLTC

SEQ ID NO: 386 - protein, 12 aa, Artificial Sequence
  Synthetic polypeptide
ILGTILGLLKGL

(19) United States
(12) Patent Application Publication
(10) Pub. No.: US 2021/0347823 A1
(43) Pub. Date: Nov. 11, 2021

(54) METHODS OF PHYSICOCHEMICAL-GUIDED PEPTIDE DESIGN AND NOVEL PEPTIDES DERIVED THEREFROM

(71) Applicants: Massachusetts Institute of Technology, Cambridge, MA (US); Fundação Universidade Federal do ABC - UFABC (Federal University of ABC Foundation), Santo André (BR)

(72) Inventors: Timothy Kuan-Ta Lu, Cambridge, MA (US); Cesar de la Fuente Nunez, Philadelphia, PA (US); Vani Xavier de Oliveira Junior, Rodovia Regis Bittencourt (BR); Marcelo Der Torossian Torres, Philadelphia, PA (US)

(73) Assignees: Massachusetts Institute of Technology, Cambridge, MA (US); Fundação Universidade Federal do ABC - UFABC (Federal University of ABC Foundation), Santo André (BR)

(21) Appl. No.: 17/277,783

(22) PCT Filed: Sep. 19, 2019
(86) PCT No.: PCT/US2019/051925

§ 371 Date: Mar. 19, 2021

Related U.S. Application Data
(60) Provisional application No. 62/734,298, filed on Sep. 21, 2018.

(51) Int. Cl. C07K 7/08; A61P 31/04; A61P 31/10

(52) U.S. Cl. CPC C07K 7/08; A61K 38/00; A61P 31/10; A61P 31/04

(57) ABSTRACT

Described herein are methods of physicochemical-guided peptide design that utilize specific functional determinants to a protein's property of interest. Also described herein are novel synthetic peptide antibiotics that have increased potency and/or decreased toxicity relative to the template peptide from which they were derived, and methods of use thereof in treating microbial infections.

### RELATED APPLICATIONS

This application claims priority under 35 U.S.C. § 119(e) to U.S. patent application No. 62/734,298, filed Sep. 21, 2018, the entire contents of which are incorporated herein by reference.

### GOVERNMENT SUPPORT

This invention was made with Government support under Grant No. HDTRA1-15-1-0050 awarded by the Defense Threat Reduction Agency. The Government has certain rights in the invention

### FIELD

Described herein are methods of physicochemical-guided peptide design that utilize specific functional determinants to a protein's property of interest. Also described herein are novel synthetic peptide antibiotics that have increased potency and/or decreased toxicity relative to the template peptide from which they were derived, and methods of use thereof in treating microbial infections.

### BACKGROUND

Drug-resistant bacteria are a major health problem worldwide (CDC Current. 114, doi: CS239559-B (2013)). Even in developed countries such as the United States, each year ˜2 million people become infected with antibiotic-resistant bacteria, resulting in at least 23,000 deaths annually (CDC Current. 114, doi:CS239559-B (2013)). Therefore, there is an urgent need to develop new therapeutics to combat drug resistance (Walsh C., Nature. 406, 775-781 (2000); Arora G. et al., Springer. doi:10.1007/978-3-319-48683-3 (2017)).

Antimicrobial peptides (AMPs) represent a promising alternative to conventional antibiotics because of their potency against difficult-to-treat infections (Mahlapuu M., et al., Front. Cell. Infect. Microbiol. 6, 1-12 (2016)), such as the ESKAPE pathogens (Enterococcus faecium, Staphylococcus aureus, Klebsiella pneumoniae, Acinetobacter baumannii, Pseudomonas aeruginosa and Enterobacter spp.) (Pendleton J. N., et al., Expert Rev. Anti. Infect. Ther. 11, 297-308 (2013)), which are relevant microorganisms for posing a clinical threat for the existing treatments due to their virulence, resistance, transmission and pathogenicity. AMPs are produced as a mechanism of defense (e.g., against infections) by virtually all living organisms. Some of these peptides exhibit broad-spectrum activity, targeting both bacterial and mammalian cells indiscriminately. However, the biological function of AMPs may be tuned by modulating biophysical features to favor specificity, selectivity (de la Fuente-Nunez C. et al., Curr. Opin. Microbiol. 37, 95-102 (2017)), potency (Melo M. N. et al., Nat. Rev. Microbiol. 7, 245-250 (2009)) and other desired biological parameters to turn these molecules into novel anti-infective agents.

### SUMMARY

As described herein, a rational peptide design strategy aimed at tuning physicochemical features involved in structure and function such as hydrophobicity, net positive charge, and helical content, was used to generate novel peptide antibiotics.

In some aspects, the disclosure relates to antimicrobial peptides. In some embodiments, the antimicrobial peptide comprises the amino acid sequence of any one of SEQ ID NOs: 2-383. In some embodiments, the antimicrobial peptide comprises the amino acid sequence of SEQ ID NO: 8, SEQ ID NO: 16, SEQ ID NO: 17, SEQ ID NO: 18, or SEQ ID NO: 21. In some embodiments, the antimicrobial peptide consists of the amino acid sequence of any one of SEQ ID NOs: 2-383. In some embodiments, the antimicrobial peptide consists of the amino acid sequence of SEQ ID NO: 8, SEQ ID NO: 16, SEQ ID NO: 17, SEQ ID NO: 18, or SEQ ID NO: 21.

In some aspects, the disclosure relates to compositions comprising an antimicrobial peptide described herein (e.g., comprising the amino acid sequence of any one of SEQ ID NOs: 2-383). In some embodiments, the composition further comprises a pharmaceutically acceptable carrier.

In yet other aspects, the disclosure relates to methods of treating a microbial infection. In some embodiments, the method comprises administering, to a subject in need of such treatment, a therapeutically effective amount of an antimicrobial peptide described herein (e.g., comprising the amino acid sequence of any one of SEQ ID NOs: 2-383). In other embodiments, the method comprises administering, to a subject in need of such treatment, a therapeutically effective amount of a composition described herein (e.g., comprising an antimicrobial peptide comprising the amino acid sequence of any one of SEQ ID NOs: 2-383).

In some embodiments, the subject is a mammal. In some embodiments, the subject is human.

In some embodiments, the antimicrobial peptide or the composition is administered orally, intravenously, intramuscularly, subcutaneously, or topically.

In some embodiments, the microbial infection comprises a bacterial, fungal, algal, viral, or protozoan infection.

In some embodiments, the microbial infection comprises a bacterial infection.

In some embodiments, the bacterial infection comprises a Gram-positive bacterium. For example, in some embodiments, the bacterial infection comprises a bacterium selected from the group consisting of a Micrococcus luteus bacterium, a Staphylococcus aureus bacterium, a Staphylococcus epidermidis bacterium, a Bacillus megaterium bacterium, and an Enterococcus faecium bacterium. In some embodiments: (a) the bacterium is a Micrococcus luteus bacterium, wherein the Micrococcus luteus bacterium is strain A270; (b) the bacterium is a Staphylococcus aureus bacterium, wherein the Staphylococcus aureus bacterium is strain ATCC29213 or ATCC12600; (c) the bacterium is a Staphylococcus epidermidis bacterium, wherein the Staphylococcus epidermidis bacterium is a strain ATCC12228; or (d) the bacterium is a Bacillus megaterium bacterium, wherein the Bacillus megaterium bacterium is a strain ATCC10778.

In some embodiments, the bacterial infection comprises a Gram-negative bacterium. For example, in some embodiments, the bacterial infection comprises a bacterium selected from the group consisting of an Escherichia coli bacterium, an Enterobacter cloacae bacterium, a Serratia marcescens bacterium, a Pseudomonas aeruginosa bacterium, a Klebsiella pneumoniae bacterium, and an Acinetobacter baumannii bacterium. In some embodiments: (a) the bacterium is an Escherichia coli bacterium, wherein the Escherichia coli bacterium is a strain SBS 363 or BL21; (b) the bacterium is an Enterobacter cloacae bacterium, wherein the Enterobacter cloacae bacterium is a strain ®-12; (c) the bacterium is a Serratia marcescens bacterium, wherein the Serratia marcescens bacterium is a strain ATCC4112; or (d) the bacterium is a Pseudomonas aeruginosa bacterium, wherein the Pseudomonas aeruginosa bacterium is a strain PA14 or PA01.

In some embodiments, the microbial infection comprises a fungal infection. In some embodiments, the fungal infection comprises a pathogenic yeast. For example, in some embodiments, the fungal infection comprises a pathogenic yeast selected from the group consisting of Candida albicans and Candida tropicalis. In some embodiments: (a) the pathogenic yeast is Candida albicans, wherein the Candida albicans is strain MDM8; or (b) the pathogenic yeast is Candida tropicalis, wherein the Candida tropicalis is strain IOC4560.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIGS. 1A-1C. Schematic of the structure-function-guided exploration approach leveraged to generate peptide antibiotics. FIG. 1A. The wasp venom derived antimicrobial peptide Polybia-CP was subjected to structure-function analysis to elucidate the determinant responsible for biological activity. FIG. 1B. Data from physicochemical properties and structure analyses was harnessed to (FIG. 1C) identify functional determinants and generate enhanced synthetic variants with therapeutic potential.

FIGS. 2A-2E. Design, physicochemical features and activity of Pol-CP—NH₂ and Ala-scan analogs. FIG. 2A. Theoretical physicochemical properties of interest of the wild type and Ala-scan analogs, where H denotes hydrophobicity, μH is the hydrophobic moment, q represents the net charge and P/N is the ratio of polar/non-polar residues in the sequence. Top to bottom, left to right the sequences correspond to SEQ ID NOs: 1-13. FIG. 2B. Schematic of the in vitro biological activity experimental design. Briefly, 10⁴ bacterial cells and serially diluted peptides (0-128 μmol L⁻¹) were added to a 96-well plate and incubated at 37° C. One day after the exposure, the solution in each well was measured in a microplate reader (600 nm) to check inhibition of bacteria compared to the untreated controls and presented as heat maps of antimicrobial activities (μmol L⁻¹) against four bacteria strains: E. coli strain BL21, S. aureus strain ATCC12600 and P. aeruginosa strains PA01, and PA14. Assays were performed in triplicates. FIG. 2C. Graph correlating MIC (μmol L⁻¹) averages vs H and (FIG. 2D) MIC (μmol L⁻¹) averages vs μH, where dots below the dashed line represent peptides with lower activity and dots above the dashed line show peptides with higher activity compared to the wild type, in which one can observe ranges of optimal activity in determined intervals of H and μH values. FIG. 2E. Bi-dimensional helical wheels representations of the wild-type indicating positions where Ala-substitution decreased (arrows in top schematic) and enhanced activity (arrows in bottom schematic) and three-dimensional representation from molecular modeling showing substitution positions in which the residues are arranged in two defined faces (hydrophobic and hydrophilic).

FIGS. 3A-3C. Physicochemical features and structure of Pol-CP—NH₂ and Ala-scan analogs. FIG. 3A. Circular dichroism (CD) spectra of Pol-CP—NH₂ and Ala-scan derivatives at 50 μmol L⁻¹ in water, PBS (pH 7.4) and TFE/Water (3:2, v:v) showing peptides transition from unstructured in water to helically structured in TFE/water. CD were recorded after four accumulations at 20° C., using a 1 mm path length quartz cell, between 260 and 190 nm at 50 nm min⁻¹, with a bandwidth of 0.5 nm. FIG. 3B. Helical fraction (f_{H}) of the peptides in each condition analyzed. FIG. 3C. MIC (μmol L⁻¹) average for each peptide against the first set of bacteria (E. coli BL21, P. aeruginosa PA01 and PA14, and S. aureus ATCC12600) in triplicates vs f_{H} in TFE/Water solution, where dots above the dashed line represent peptides with lower activity and dots below the dashed line show peptides with higher activity compared to the wild type. Optimal activity is reached in most of the cases for f_{H} values higher than the wild type.

FIG. 4A-4C. Physicochemical features and structure of Pol-CP—NH₂ and second-generation analogs. FIG. 4A. Theoretical physicochemical properties of interest of the wild type and the newly designed derivatives, where H denotes hydrophobicity, μH is the hydrophobic moment, q represents the net charge and P/N is the ratio of polar/non-polar residues in the sequence. Lysine modifications led to increased net positive charge and glutamic acid modifications led to decreased net positive charge (see table to the right). The impact of modification with hydrophobic/aliphatic residues was also analyzed (see table to the right). Top to bottom, left to right the sequences correspond to SEQ ID NOs: 1, 14-21. FIG. 4B. Circular dichroism spectra of the peptides at 50 μmol L⁻¹ in water, MeOH/Water (1:1, v:v), PBS (pH 7.4), POPC (10 mmol L⁻¹), POPC:DOPE (3:1, 10 mmol L⁻¹), POPC:POPG (3:1, 10 mmol L⁻¹), SDS (20 mmol L⁻¹), TFE/Water (2:3, 3:2, 4:1, v:v) showing peptides transition from unstructured in water to helically structured in TFE/water. CD were recorded after four accumulations at 20° C., using a 1 mm path length quartz cell, between 260 and 190 nm at 50 nm min⁻¹, with a bandwidth of 0.5 nm. FIG. 4C. Helical fraction (f_{H}) of the peptides in each condition analyzed.

FIGS. 5A-5D. Antimicrobial activity of second-generation library of synthetic peptides. FIG. 5A. In vitro activity of Pol-CP—NH₂ and second generation of analogs against Gram-positive bacteria (Micrococcus luteus, Staphylococcus aureus, Staphylococcus epidermidis and Bacillus megaterium), Funghi (Candida albicans and Candida tropicalis) and Gram-negative bacteria (Escherichia coli, Enterobacter cloacae and Serratia marcescens). Experiments performed in triplicates. FIG. 5B. MIC (μmol L⁻¹) average vs f_{H} in TFE/Water solution. FIG. 5C. Graph correlating MIC (μmol L⁻¹) averages vs H and (FIG. 5D) MIC (μmol L⁻¹) averages vs μH, where dots above the dashed line represent peptides with lower activity and dots below the dashed line show peptides with higher activity compared to the wild-type, in which one can observe ranges of optimal activity in determined intervals of H and μH values.

FIGS. 6A-6B. Hemolysis and resistance to protease-mediated degradation of engineered peptides. FIG. 6A. Schematic of experimental design and hemolytic assay results of Pol-CP—NH₂ and derivatives, where hemolytic activity was evaluated by incubating the peptides (0.1-100 μmol L⁻¹) with human red blood cells in PBS at room temperature for 1 h. Experiments were performed in triplicate, (*p<0.05). FIG. 6B. Resistance to degradation of Pol-CP—NH₂ and analogs exposed to fetal bovine serum (FBS) proteases for 6 h. Experiments were done in triplicate.

FIGS. 7A-7B. Cytotoxicity of engineered peptides. FIG. 7A. Schematic of the experimental design for cytotoxicity assays of Pol-CP—NH₂ and derivatives against HEK293 human embryonic kidney cells. Briefly, cells were cultured in DMEM medium supplemented with FBS and antibiotics at 37° C. and 5% CO₂. FIG. 7B. Results obtained by seeding HEK293 50,000 cells and incubating with peptides' solution (0-64 μmol L⁻¹) at 37° C. for 48 h. Cell viability was measured by MTS assay. All experiments were performed in triplicate.

FIGS. 8A-8D. In vivo activity of Pol-CP—NH₂ and its analogs. FIG. 8A. Schematic of the experimental design. Briefly, the back of mice was shaved and an abrasion was generated to damage the stratum corneum and the upper layer of the epidermis. Subsequently, an aliquot of 50 μL containing 5×10⁷ CFU of P. aeruginosa in PBS was inoculated over each defined area. One day after the infection, peptides (4 μmol L⁻¹) were administered to the infected area. Animals were euthanized and the area of scarified skin was excised two and four days post-infection (FIG. 8B) homogenized using a bead beater for 20 minutes (25 Hz), and serially diluted for CFU quantification (****p<0.0001). FIG. 8C. Mouse body weight measurements throughout the experiment normalized by the body weight of non-infected mice. The wild type peptide and the most active analog ([Lys]⁷-Pol-CP—NH₂) were used at 64 mol L⁻¹, where infection and CFU quantification were performed as described in (FIG. 8B), the body weight of mice treated with peptide did not change significantly compared to untreated mice. FIG. 8D. Longer experiment (four days) using a higher concentration (64 μmol L⁻¹) of peptides Pol-CP—NH₂ and [Lys]'-Pol-CP—NH₂ (****p<0.0001).

FIG. 9. Helical wheel representations of the Ala-scan Pol-CP—NH2 analogs generated using the Heliquest server (Gautier R. et al., Bioinformatics 24, 2101-2102 (2008)) considering theoretical helical structure and physicochemical properties derived from the amphipathic distribution. The black arrows inside the helical wheel projection of each peptide represent their hydrophobic moment vector, whose magnitude is indicated by the size of the arrows.

FIGS. 10A-10B. FIG. 10A. Schematic of the in vitro CFU count setup to assess antimicrobial activity of Pol-CP—NH₂ and Ala-scan analogs. Briefly, 10⁴ bacterial cells and serially diluted (0-64 μmol L⁻¹) peptides were added to a 96-well plate and incubated at 37° C. One day after the exposure, the solution in each well was 10-fold diluted seven times and the serial dilutions were plated in agar plates, which were incubated for 22 h at 37° C. FIG. 10B. Next, bacterial colonies were counted. All assays were performed in triplicate (error bars=standard error of the mean, ns=statistically not significant, *p<0.05, **p<0.005, ***p<0.001, ****p<0.0001).

FIGS. 11A-11B. FIG. 11A. Graphical representation of residues movement of Pol-CP-NH₂ and Ala-scan analogs from molecular dynamics simulations in water and TFE/water (3:2, v:v), yielding root mean square deviation (RMSD), root mean square fluctuation (RMSF) and radius of gyration (Rg) after 100 ns. FIG. 11B. Three-dimensional theoretical structures snapshots of Pol-CP—NH₂ and Ala-scan derivatives during 100 ns of molecular dynamics simulation. N-terminus of each peptide is always at the bottom.

FIG. 12. Helical wheel representations of the second generation of Pol-CP—NH2 analogs generated using the Heliquest server (Gautier R. et al., Bioinformatics 24, 2101-2102 (2008)) considering theoretical helical structure and physicochemical properties derived from the amphipathic distribution. The arrows inside the helical wheel projection of each peptide represent their hydrophobic moment vector, whose magnitude is indicated by the size of the arrows.

FIGS. 13A-13B. FIG. 13A. Considerations for each one of the second generation analogs designed synthesized in this work to check the importance of different kinds of substitutions and how well can the optimal hotspots describe activity propensities and (FIG. 13B) In vitro antimicrobial activity of the lead peptides from the second generation of Pol-CP—NH₂ derived agents. Serially diluted (0-128 μmol L⁻¹) peptides were added to a 96-well plate containing 10⁴ bacterial cells in each well and incubated at 37° C. for 24 h. After the exposure, the solution in each well was measured in a microplate reader (600 nm) to check inhibition of bacteria compared to the untreated controls and presented as heat maps of antimicrobial activities (μmol L⁻¹) against four bacteria strains: Escherichia coli strain BL21, S. aureus strain ATCC12600 and P. aeruginosa strains PA01 and PA14. Assays were performed in triplicate. In FIG. 13A, top to bottom, left to right the sequences correspond to SEQ ID NOs: 1, 14-21.

FIGS. 14A-14B. FIG. 14A. Graphical representation of residues movement of Pol-CP—NH₂ and second generation of analogs from molecular dynamics simulations in water and TFE/water (3:2, v:v), yielding root mean square deviation (RMSD), root mean square fluctuation (RMSF) and radius of gyration (Rg) after 100 ns. FIG. 14B. Three-dimensional theoretical structures snapshots of Pol-CP—NH₂ and derivatives during 100 ns of molecular dynamics simulation. N-terminus of each peptide is always at the bottom.

### DETAILED DESCRIPTION

Despite some obstacles, such as short half-life in blood stream-like environments of small linear natural peptides and intrinsic bacterial resistance (i.e. membrane modifications efflux and proteolytic degradation) to certain host defense peptides (Andersson D. I. et al, Drug Resist. Updat. 26, 43-57 (2016)), AMPs are a promising alternative to conventional antibiotics because of their unique diversity of peptide sequences. Their sequence space is almost unlimited, and a wide range of amino acids is available in nature (Perumal Samy R. et al., Biochem. Pharm. 134, (2017)). Biological evolution has selected AMPs with certain sequence biases; however, even minor changes to these sequences enabled by peptide engineering may yield unprecedented biological function. The most widely studied class of AMPs is that comprising the linear cationic amphipathic AMPs (Hancock R. E. W. Expert Opin. Investig. Drugs 9, 1723-1729 (2000)), which shift from coiled to helical structures (Lifson S. and Roig A. J. Chem. Phys. 34, 1963-1974 (1961); Zimm B. H. and Bragg J. K. J. Chem. Phys. 31, 526-535 (1959)) when the peptide comes into contact with membranes of microorganisms.

Most AMPs act by disrupting the cytoplasmic membrane of microorganisms in ways (Nguyen L.T. et al., Trends Biotechnol. 29, 464-472 (2011)) that are not necessarily exclusive of one another. Important mechanisms of action of AMPs are carpet-like, barrel stave, or toroidal pore formation (Brogden K. A. Nat. Rev. Microbiol. 3, 238 (2005)). Other specific or general mechanisms have been described, such as membrane thickening/thinning (Lohner K. Gen. Physiol. Biophys. 28, 105-116 (2009)), charged lipid clustering (Epand R. M. and Epand R. F. J. Pept. Sci. 17, 298-305 (2011)), nucleic acids targeting (Brogden K. A. Nat. Rev. Microbiol. 3, 238 (2005)), anion carriers (Rokitskaya T. I. et al., Biochim. Biophys. Acta—Biomembr. 1808, 91-97 (2011)), electroporation (Chan D. I. et al., Biochim. Biophys. Acta—Biomembr. 1758, 1184-1202 (2006)), non-lytic membrane depolarization (Gifford J. L. et al., Cell. Mol. Life Sci. 62, 2588-98 (2005)), and non-bilayer intermediates (Haney E. F. et al., Chem. Phys. Lipids 163, 82-93 (2010)). However, some AMPs antimicrobial mode of action include targeting key cellular processes and metabolic pathways (Le C. F. et al., Sci. Rep. 6, 26828 (2016); Huang N. et al., Tumor Biol. 39, 1010428317708532 (2017)) including DNA and protein synthesis (Park C. B. et al., Biochem. Biophys. Res. Commun. 244, 253-257 (1998); Krizsan A. et al., Eur. J. Chem. Biol. 16, 2304-2308 (2015)), protein folding, enzymatic activity and cell wall synthesis (de Kruijff B. et al., Prostaglandins, Leukot. Essent. Fat. Acids 79, 117-121 (2008)), cell division (Subbalakshmi C. and Sitaram N. FEMS Microbiol. Lett. 160, 91-96 (1998)), RNA synthesis (Haney E. F. et al., Biochim. Biophys. Acta—Biomembr. 1828, 1802-1813 (2013)), inactivation of chaperone proteins necessary for proper folding, and even targeting mitochondria (da Costa J. P. et al., Appl. Microbiol. Biotechnol. 99, 2023-2040 (2015)).

Insects such as wasps, scorpions and spiders are rich sources of linear cationic amphipathic AMPs (Perumal Samy R. et al., Biochem. Pharm. 134, (2017)). The South American social wasp Polybia paulista has a large variety of peptides in its venom, each of which has a different biological function (Lee S. H. et al., Toxins (Basel). 8, 1-29 (2016)). Among them, the mastoparan class is a well-known group of chemotactic peptides having inflammatory and antimicrobial activities (Lee S. H. et al., Toxins (Basel). 8, 1-29 (2016)). Souza et al. reported a 12-residue cationic amphipathic mastoparan-like AMP, polybia-CP (Pol-CP—NH₂: Ile-Leu-Gly-Thr-Ile-Leu-Gly-Leu-Leu-Lys-Ser-Leu-NH₂ (SEQ ID NO: 1)), which presents poor activity against Gram-negative bacteria, higher activity against Gram-positive bacteria, and toxicity towards human cells (Souza B. M. et al., Peptides 26, 2157-2164 (2005)). The lower activity of Pol-CP—NH₂ against Gram-negative bacteria was attributed to its low predicted helical content and to the presence of a hydrophilic serine residue next to its C-terminus, a residue that is not present in this position in other mastoparan-like peptides from the same wasp venom, such as protonectin and polybia-MPI (Souza B. M. et al., Peptides 26, 2157-2164 (2005)).

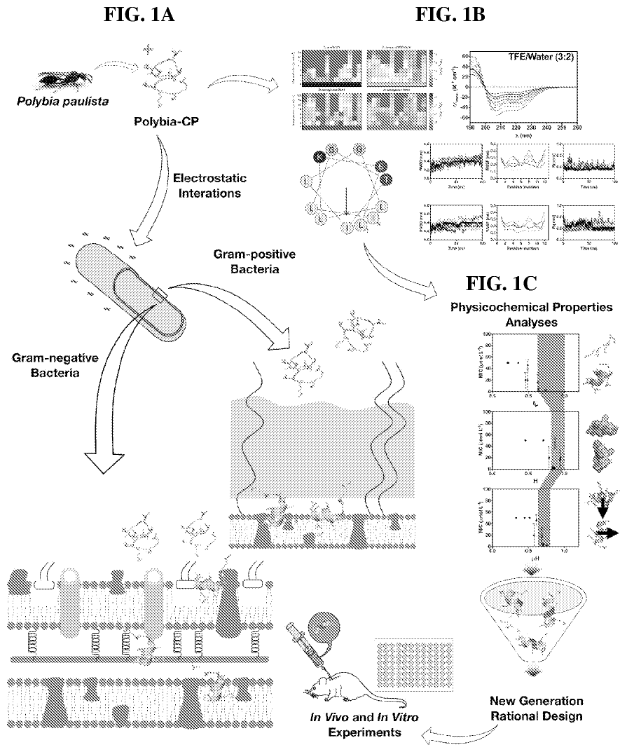

As described herein, a rational peptide design strategy aimed at tuning physicochemical features involved in structure and function such as hydrophobicity, net positive charge, and helical content, was used herein to improve the antimicrobial activity of Pol-CP—NH₂ and to generate novel peptide antibiotics (FIGS. 1A-1C).

As such, in some aspects, the disclosure relates to synthetic antimicrobial peptides (i.e., consisting of an amino acid sequence that is not found in nature). In some embodiments, the antimicrobial peptide comprises the amino acid sequence of any one of SEQ ID NOs: 2-383. In some embodiments, the antimicrobial peptide comprises the amino acid sequence of SEQ ID NO: 8, SEQ ID NO: 16, SEQ ID NO: 17, SEQ ID NO: 18, or SEQ ID NO: 21. In some embodiments, the antimicrobial peptide consists of the amino acid sequence of any one of SEQ ID NOs: 2-383. In some embodiments, the antimicrobial peptide consists of the amino acid sequence of SEQ ID NO: 8, SEQ ID NO: 16, SEQ ID NO: 17, SEQ ID NO: 18, or SEQ ID NO: 21.

In some aspects, the disclosure relates to compositions comprising an antimicrobial peptide described herein (e.g., an antimicrobial peptide comprising or consisting of any one of SEQ ID NOs: 2-383).

In some embodiments, each of the antimicrobial peptides in the composition are chemically identical.

In other embodiments, the composition comprises a plurality of antimicrobial peptides comprising chemically distinct antimicrobial peptides. For example, in some embodiments, a composition comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more chemically distinct antimicrobial peptides. In some embodiments, a composition comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, or at least 50 chemically distinct antimicrobial peptides. In some embodiments, a composition comprises 2-3, 2-4, 2-5, 2-6, 2-7, 2-8, 2-9, 2-10, 2-11, 2-12, 2-13, 2-14, 2-15, 2-16, 2-17, 2-18, 2-19, 2-20, 3-4, 3-5, 3-6, 3-7, 3-8, 3-9, 3-10, 3-11, 3-12, 3-13, 3-14, 3-15, 3-16, 3-17, 3-18, 3-19, 3-20, 4-5, 4-6, 4-7, 4-8, 4-9, 4-10, 4-11, 4-12, 4-13, 4-14, 4-15, 4-16, 4-17, 4-18, 4-19, 4-20, 5-6, 5-7, 5-8, 5-9, 5-10, 5-11, 5-12, 5-13, 5-14, 5-15, 5-16, 5-17, 5-18, 5-19, 5-20, 5-25, 5-30, 5-35, 5-40, 5-45, 5-50, 10-11, 10-12, 10-13, 10-14, 10-15, 10-16, 10-17, 10-18, 10-19, 10-20, 10-25, 10-30, 10-35, 10-40, 10-45, or 10-50 chemically distinct antimicrobial peptides.

In some embodiments, each of the antimicrobial peptides in the composition comprises an amino acid sequence selected from the group consisting of any one of SEQ ID NOs: 2-383. In some embodiments, a subset of the antimicrobial peptides in the composition comprises an amino acid sequence selected from the group consisting of any one of SEQ ID NOs: 2-383.

The compositions described herein may further comprise a pharmaceutically-acceptable carrier. Generally, for pharmaceutical use, the composition may be formulated as a pharmaceutical preparation or composition comprising at least one active unit (i.e., at least one antimicrobial peptide) and at least one pharmaceutically acceptable carrier, diluent or excipient, and optionally one or more further pharmaceutically active compounds. Such a formulation may be in a form suitable for oral administration, for parenteral administration (such as by intravenous, intramuscular or subcutaneous injection or intravenous infusion), for topical administration, for administration by inhalation, by a skin patch, by an implant, by a suppository, etc. Such administration forms may be solid, semi-solid or liquid, depending on the manner and route of administration. For example, formulations for oral administration may be provided with an enteric coating that will allow the formulation to resist the gastric environment and pass into the intestines. More generally, formulations for oral administration may be suitably formulated for delivery into any desired part of the gastrointestinal tract. In addition, suitable suppositories may be used for delivery into the gastrointestinal tract. Various pharmaceutically acceptable carriers, diluents and excipients useful in therapeutic compositions are known to the skilled person.

As used herein, the term “pharmaceutically-acceptable carrier” refers to one or more compatible solid or liquid filler, diluents or encapsulating substances which are suitable for administration to a human or other subject contemplated by the disclosure. As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers (e.g., antioxidants), gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences (1990), incorporated herein by reference). Except insofar as any conventional carrier is incompatible with the active ingredient, its use in the therapeutic or pharmaceutical compositions is contemplated.

In yet other aspects, the disclosure relates to methods of treating a microbial infection in a subject in need of such a treatment. In some embodiments the subject is a mammal, such as a human.

In some embodiments, the method comprises administering, to a subject in need of such treatment, a therapeutically effective amount of an antimicrobial peptide as described herein (or a composition comprising an antimicrobial peptide as described herein). The antimicrobial peptide (or composition comprising an antimicrobial peptide) may be administered orally, intravenously, intramuscularly, subcutaneously, or topically. Alternatively or in addition, administration may be by inhalation, by a skin patch, by an implant, by a suppository, etc. Additional modes of administration are known to those having ordinary skill in the art.

As used herein the term “therapeutically effective” applied to an amount refers to that quantity of a compound or composition that is sufficient to result in a desired activity upon administration to a subject in need thereof. For example, the term “therapeutically effective” refers to a quantity of a compound or pharmaceutical composition that is sufficient to delay the manifestation, arrest the progression, relieve or alleviate at least one symptom associated with a microbial infection.

A microbial infection may comprise a bacterial infection, a fungal infection, an algal infection, a viral infection, a protozoan infection, or a combination thereof. Examples of bacterial infections, fungal infections, algal infections, viral infections, and protozoan infections are known to those having ordinary skill in the art.

In some embodiments, a microbial infection comprises a bacterial infection.

The bacterial infection may comprise a Gram-positive bacterium, such as a Micrococcus luteus bacterium, a Staphylococcus aureus bacterium, a Staphylococcus epidermidis bacterium, a Bacillus megaterium bacterium, or an Enterococcus faecium bacterium. Additional infectious Gram-positive bacterium are known to those having ordinary skill in the art. In some embodiments, a bacterial infection comprises: (a) a Micrococcus luteus bacterium, wherein the Micrococcus luteus bacterium is strain A270; (b) a Staphylococcus aureus bacterium, wherein the Staphylococcus aureus bacterium is strain ATCC29213 or ATCC12600; (c) a Staphylococcus epidermidis bacterium, wherein the Staphylococcus epidermidis bacterium is a strain ATCC12228; (d) a Bacillus megaterium bacterium, wherein the Bacillus megaterium bacterium is a strain ATCC10778; or (e) a combination thereof.

Alternatively or in addition, the bacterial infection may comprise a Gram-negative bacterium, such as an Escherichia coli bacterium, an Enterobacter cloacae bacterium, a Serratia marcescens bacterium, a Pseudomonas aeruginosa bacterium, a Klebsiella pneumoniae bacterium, or an Acinetobacter baumannii bacterium. Additional infectious Gram-negative bacterium are known to those having ordinary skill in the art. In some embodiments, a bacterial infection comprises: (a) an Escherichia coli bacterium, wherein the Escherichia coli bacterium is a strain SBS 363 or BL21; (b) an Enterobacter cloacae bacterium, wherein the Enterobacter cloacae bacterium is a strain ®-12; (c) a Serratia marcescens bacterium, wherein the Serratia marcescens bacterium is a strain ATCC4112; (d) a Pseudomonas aeruginosa bacterium, wherein the Pseudomonas aeruginosa bacterium is a strain PA14 or PA01; or (e) a combination thereof.

Alternatively or in addition, a microbial infection may comprise a fungal infection. The fungal infection may comprise a pathogenic yeast, such as Candida albicans or Candida tropicalis. Additional pathogenic yeast are known to those have ordinary skill in the art. In some embodiments, the fungal infection comprises: (a) Candida albicans, wherein the Candida albicans is strain MDM8; (b) Candida tropicalis, wherein the Candida tropicalis is strain IOC4560; or (c) a combination thereof.

### EXAMPLES

### Methods

Solid-phase peptide synthesis (SPPS), Purification and Analysis. Ala-scan analogs were acquired from Biopolymers and the second generation of peptides was synthesized on a peptide synthesizer (PS3—Sync Technologies) using the fluoromethyloxycarbonyl (Fmoc) strategy, Rink Amide resin, with a substitution degree of 0.52 mmol g⁻¹. Deprotection steps were carried out by treatment with 4-methylpiperidine in dimethylformamide (4-MePip/DMF, 1:4, v/v) for 40 minutes. Amino acid coupling steps were accomplished by treating the deprotected amino acyl-resin with 4-fold molar excess of the Fmoc-protected amino acid, activated by N,N,N′,N′-tetramethyl-O-(1H-benzotriazol-1-yl)-uranium hexafluorophosphate (HBTU) in DMF, for 30 minutes at room temperature. Each step was followed by a washing procedure with DMF to favor resin swelling, elimination of excess reagents and byproducts, leading to the peptidyl-resin.

Dry-protected peptidyl-resin was exposed to trifluoroacetic acid (TFA)/Anisole/Water (95:2.5:2.5, v/v/v) for two hours at room temperature. The crude deprotected peptides were precipitated with anhydrous diethyl ether, filtered from the ether-soluble products, extracted from the resin with 60% ACN (acetonitrile) in water and lyophilized.

The crude lyophilized peptides were then purified by preparative reverse-phase high-performance liquid chromatography (RP-HPLC) in 0.1% TFA/90% ACN in water (A/B) on a Delta Prep 600 (Waters Associates). Briefly, the peptides were loaded onto a Phenomenex C₁₈ (21.2 mm×250 mm, 15 μm particles, 300 Å pores) column at a flow rate of 10.0 mL min⁻¹ and eluted using a linear gradient (0.33% B/min slope), with detection at 220 nm. Selected fractions containing the purified peptides were pooled and lyophilized. Purified peptides were characterized by liquid-chromatography electrospray-ionization mass spectrometry (LC/ESI-MS).

LC/ESI-MS data were obtained on a Model 6130 Infinity mass spectrometer coupled to a Model 1260 HPLC system (Agilent) using a Phenomenex Gemini C₁₈ column (2.0 mm×150 mm, 3.0 μm particles, 110 Å pores). Solvent A was 0.1% TFA in water, and solvent B was 90% ACN in solvent A. Elution with a 5-95% B gradient was performed over 20 min, 0,2 mLmin⁻¹ flow and peptides were detected at 220 nm. Mass measurements were performed in a positive mode with the following conditions: mass range between 100 to 2500 m/z, ion energy of 5.0 V, nitrogen gas flow of 12 L min⁻¹, solvent heater of 250° C., multiplier of 1.0, capillary of 3.0 kV and cone voltage of 35 V.

Circular dichroism (CD) spectroscopy. CD experiments were performed on a J-815 Circular Dichroism Spectropolarimeter (Jasco). Far-UV CD spectra were recorded after four accumulations at 20° C., using a 1 mm path length quartz cell, between 260 and 190 nm at 50 nm min⁻¹ with a band width of 0.5 nm. All peptides were analyzed in water, PBS (pH 7.4), MeOH/water (1:1; v:v), TFE/Water (2:3, 3:2 and 4:1; v:v), 10 mmol L⁻¹ POPC, 10 mmol L⁻¹ POPC:DOPE (3:1) and 10 mmol L⁻¹ POPC:POPG (3:1). The large unilamellar vesicles (POPC, POPC:DOPE and POPC:POPG) preparation was by the formation of a lipid film of the desired composition on the walls of a test tube from a lipid stock solution in chloroform, dried with a stream of N₂ and kept in a vacuum for 1 h. The lipid film was resuspended in a buffer solution (10 mmol L⁻¹ PBS, pH 7.4), and vortexed to form multilamellar vesicles. This lipid dispersion was extruded at least 21 times through a polycarbonate membrane with a pore size of 100 nm to yield the large unilamellar vesicles. The peptide concentration was 50 [symbol] mol L⁻¹. A Fourier transform filter was applied to minimize background effects.

Microorganisms. The following strains were used: Micrococcus luteus A270, Staphylococcus aureus ATCC29213, Staphylococcus epidermidis ATCC12228, Bacillus megaterium ATCC10778 Escherichia coli SBS 363, Enterobacter cloacae ®-12, Serratia marcescens ATCC4112, Candida albicans MDM8, Candida tropicalis IOC4560 from Instituto Butanta, Sao Paulo, Brazil, and Escherichia coli BL21, Pseudomonas aeruginosa PA14, Pseudomonas aeruginosa PA01 and Staphylococcus aureus ATCC12600 from Synthetic Biology Group at MIT.

MIC assays. The MIC assays were performed using the broth microdilution method (Wiegand I. et al., Protoc. 3, 163-175 (2008); de la Fuente-Núñez C. et al., Antimicrob. Agents Chemother. 56, 2696-2704 (2012)) in sterile 96-well polypropylene microtiter plates. Peptides were added to the plate as solutions in BM2 minimal medium in concentrations ranging from 0 to 128 [symbol] mol L⁻¹, and the bacteria were inoculated at a final concentration of 5×10⁵ CFU mL⁻¹ per well. The plates were incubated at 37° C. for 24 h. The MIC was defined as the lowest concentration of compound at which no growth was observed. Additional liquid growth inhibition assays were done in Peptone Broth (PB, 0.5% NaCl, 1% Peptone at pH 7.4) and Potato Dextrose Broth (Invitrogen) were used for antibacterial and antifungal assays, respectively. Briefly, bacteria or fungi were incubated with serial dilutions of polybia-CP and analogs (50-0.09 μmol L⁻¹) in a 96-well microplate at 37° C. The microbial growth was assessed by measurements in a model 354 Multiskan Ascent microplate reader at A₅₉₅ₙₘ, after 18 and 24 h (bacteria and fungi, respectively) incubation on a model 347CD FANEM incubator. MIC was defined as the minimal inhibitory concentration that prevents 100% of the bacterial growth. All assays were done in triplicate.

Bacterial Killing Experiments. Killing experiments involved performing 1:10,000 dilutions of overnight cultures of E. coli BL21, S. aureus ATCC12600, P. aeruginosa PA01 and PA14 in the absence or presence of increasing concentrations of Pol-CP—NH₂ derivatives (0-64 [symbol] mol L⁻¹). After 24 h of treatment, 10-fold serial dilutions were performed, bacteria were plated on LB agar plates (E. coli BL21 and S. aureus ATCC12600) and Pseudomonas Isolation Agar (P. aeruginosa PA01 and PA14) and allowed to grow overnight at 37° C. after which colony forming unit (CFU) counts were recorded, according to Wiegand et al. (Wiegand I. et al., Protoc. 3, 163-175 (2008)).

Hemolytic Activity Assays. Human erythrocytes were collected and washed three times by centrifugation at 300×g with PBS (pH 7.4). After the last centrifugation, the cells were resuspended in PBS pH 7.4. Aliquots at a concentration of 0.1 to 100 [symbol] mol L⁻¹ of the peptides were added to the 96-well microplate, where in each well containing 50 [symbol] L of a suspension of erythrocytes to 0.4% in a phosphate saline buffer (10 mmol L⁻¹ Na₂HPO₄, 1.8 mmol L⁻¹ K₂HPO₄, pH 7.4, 137 mmol L⁻¹ NaCl and 2.7 mmol L⁻¹KCl). After that, the samples were incubated at room temperature for 1 h. Hemolysis was determined by reading absorbance at 405 nm of each well in a bed of plates. 1% SDS in PBS solution was used as positive control (Shalel S. et al., J. Colloid Interface Sci. 252, 66-76 (2002); Love L. J. Cell. Comp. Physiol. 36, 133-148 (1950)) and as negative control was used PBS only. MHC was defined as the maximal non-hemolytic concentration.

Stability Assays. The stability assay was performed with GIBCO fetal bovine serum diluted to 25% in water. 20 [symbol] L of a 10 mg mL⁻¹ peptide solution was added to 1 mL 25% serum solution and kept at 37° C. The experiments were made in triplicate and 100 [symbol] L aliquots were taken at 0, 0.5, 1, 2, 4 and 6 h. 10 [symbol] L of TFA was added to the aliquots and the new solution was kept at 5° C. for 10 min, after that it was centrifuged at 14,000 rpm for 15 min, according to Powell et al. (Powell M. F. et al., Pharm. Res. 10, 1268-1273 (1993)). The reaction kinetics was followed by liquid chromatography and the percentage of remaining peptide was calculated by integrating the peptide peak area.

Cytotoxicity assays. Human embryonic kidney 293 (HEK 293) cells were cultured in Dulbecco's Modified Eagle Medium (DMEM) supplemented with 10% Fetal Bovine Serum (FBS) and 1% penicillin-streptomycin at 37° C. in 5% CO₂. The day before treatment, 50,000 HEK 293 cells were seeded into each well in 96-well plates. The peptides were added at concentrations ranging from 0 to 64 [symbol] mol L⁻¹ and 48 h after exposure, cell viability was measured by means of MTS (dimethylthiazol-carboxymethoxyphenyl-sulfophenyl-tetrazolium) assay. Experiments were performed in triplicate for each condition.

Scarification Skin Infection Mouse Model. P. aeruginosa strain PA14 was grown to an optical density at 600 nm (OD₆₀₀) of 1 in tryptic soy broth (TSB) medium. Subsequently cells were washed twice with sterile PBS (pH 7.4, 13,000 rpm for 1 minute), and resuspended to a final concentration of 5×10⁷ CFU/50 [symbol] L. To generate skin infection, female CD-1 mice (6 weeks old) were anesthetized with isoflurane and had their backs shaved. A superficial linear skin abrasion was made with a needle in order to damage the stratum corneum and upper-layer of the epidermis. Five minutes after wounding, an aliquot of 50 [symbol] L containing 5×10⁷ CFU of bacteria in PBS was inoculated over each defined area containing the scratch with a pipette tip. One day after the infection, peptides were administered to the infected area. Animals were euthanized and the area of scarified skin was excised two and four days post-infection, homogenized using a bead beater for 20 minutes (25 Hz), and serially diluted for CFU quantification. Two independent experiments were performed with 4 mice per group in each condition. Statistical significance was assessed using a one-way ANOVA.

Molecular Modeling. Molecular modeling studies were carried out according to four successive steps: (i) selection of a template structure; (ii) alignment between the template and target sequences; (iii) construction of atomic coordinates; and (iv) validation of the lowest free energy theoretical models. Initially, Blastp was performed and a fragment from the structure of a methyltransferase (chain A) (PDB entry: 3SSM) (Akey D. L. et al., J. Mol. Biol. 413, 438-450 (2011)) was select as template, taking into account parameters such as identity, coverage and e-value. All target sequences were individually aligned to the template and further submitted to comparative modeling simulations on MODELLER v. 9.17 (Fiser A. and Šali A. Academic Press. 374, 461-491 (2003)). A total of 100 models were generated for each peptide and ranked according to their free energy scores (DOPE score). The lowest free energy models for each peptide were validated regarding their stereochemistry and fold quality on PROCHECK (Laskowski R. A. et al., J. Appl. Crystallogr. 26, 283-291 (1993)) and ProSA-web servers (Wiederstein M. and Sippl M. J. Nucleic Acids Res. 35, W407-W410 (2007)). Finally, the validated structures were visualized and analyzed using PyMOL v. 1.8 (The PyMOL Molecular Graphics System, Version 2.0 Schrödinger, LLC).

Molecular Dynamics. Molecular dynamics simulations were conducted in hydrophilic environment (water) and in a mixture of 60% TFE/water (v/v). The GROMOS 43a1 force field (Lindahl E. et al., Mol. Model. Annu. 7, 306-317 (2001)) was used and the simulation and analysis performed using the computational package GROMACS 5.0.4 (Abraham M. J. et al., SoftwareX 1-2, 19-25 (2015)). As initial structures, the validated models obtained from molecular modeling simulations were immersed in cubic boxes containing single point charge (SPC) water molecules. Simulations in 60% TFE were also performed in cubic boxes, the peptides immersed in SPC water molecules, followed by the insertion of TFE molecules until the ideal concentration was reached. Chloride ions (Cl⁻) were also added to neutralize the system's charge. Moreover, the LINCS algorithm was used to link all the atom bond length. Particle Mesh Ewald (PME) was also used for electrostatic corrections, with a radius cut-off of 1.4 nm to minimize the computational simulation time. The same radius cut off was also used for van der Waals interactions. The list of neighbors of each atom was updated every 10 simulation steps of 2 fs each. A conjugate gradient (2 ns) and the steepest descent algorithms (2 ns) were implemented for energy minimization. After that, the systems underwent a normalization of pressure and temperature, using the integrator stochastic dynamics, 2 ns each. The systems with minimized energy and balanced temperature and pressure was carried out using a step of position restraint, using the integrator Molecular Dynamics (MD), for 2 ns. The simulations were carried out during 100 ns at 27° C. in silico, aiming to understand the structural conformation of the peptide more nearly to that observed in vitro bioassays. All simulations were programmed in triplicate.

### EXAMPLE 1

### Ala-Scan (Alanine-Scan) Screening of Pol-CP—NH₂ Sequence, and Structural Studies

The first generation of peptides was designed to evaluate the role of the side chain of each residue in biological function, and to determine how substitutions to the side chain groups of each residue would alter structural and physicochemical features when compared to those of the helical wild-type peptide Pol-CP—NH₂. Because Ala presents the smallest side chain among all-natural chiral amino acids, it was chosen to conserve the backbone size and to evaluate the effect of the native side chains on both structure and activity.

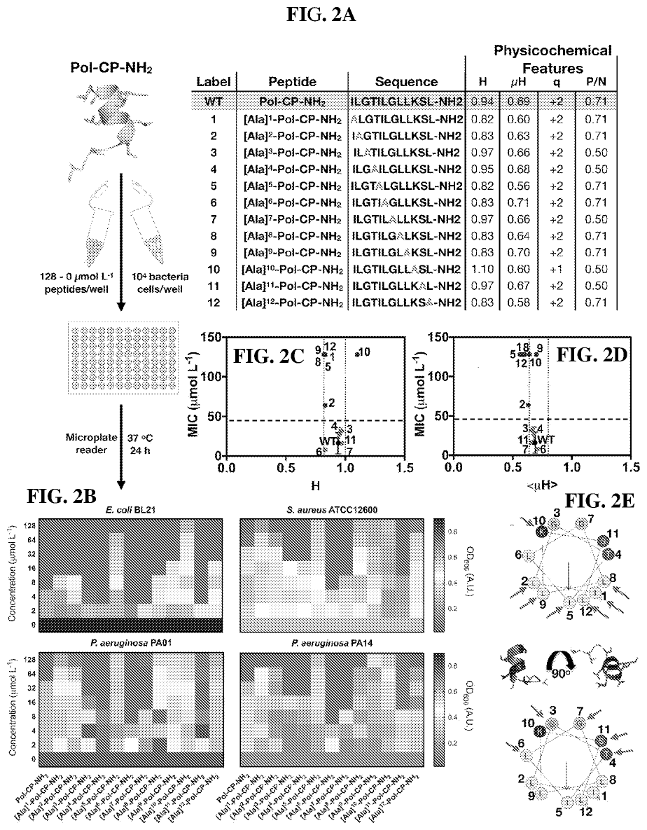

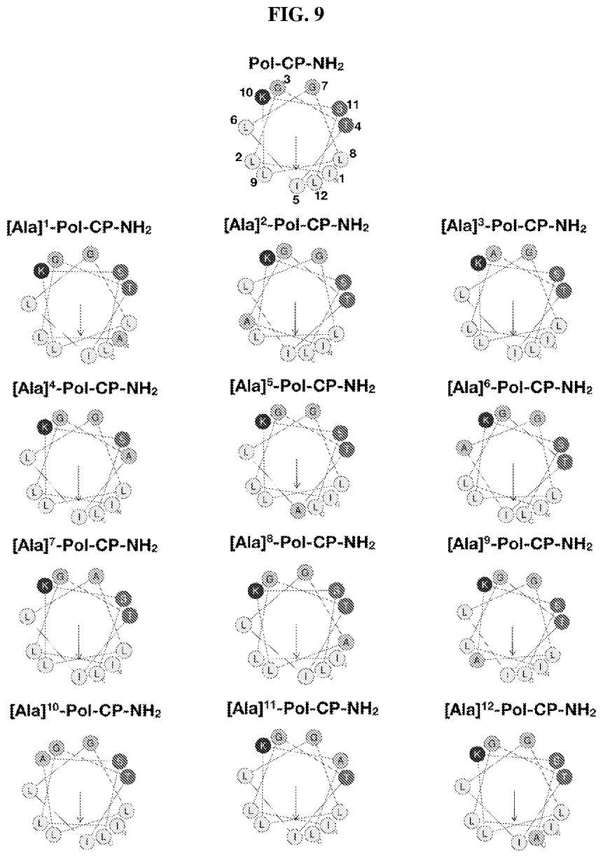

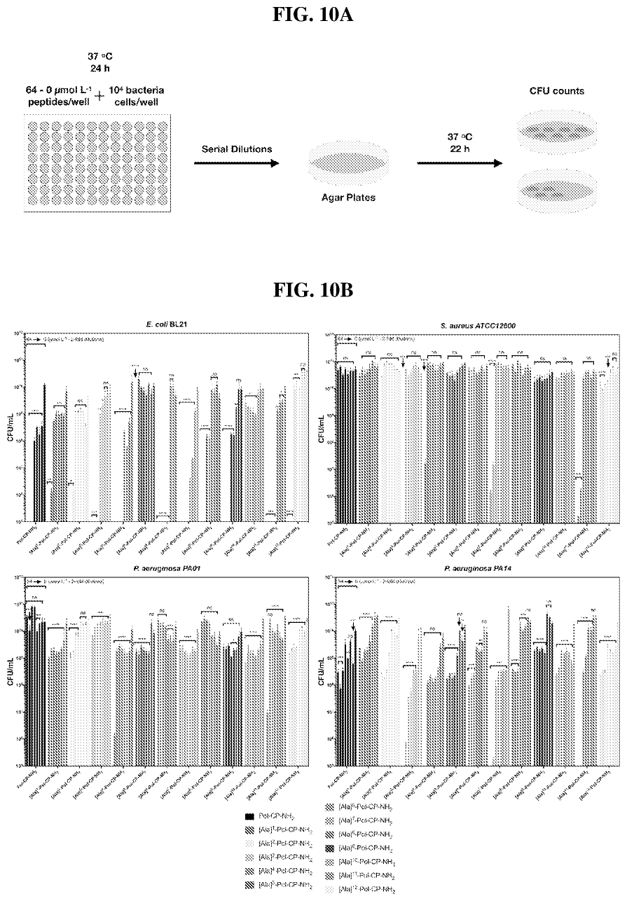

First, theoretical values of physicochemical features such as hydrophobicity, hydrophobic moment, and net positive charge were calculated, and helical wheels were generated using the Heliquest webserver (Gautier R. et al., Bioinformatics 24, 2101-2102 (2008)) (FIG. 2A and FIG. 9). Hydrophobicity values produced by the server were compared with retention times obtained by the analyses of the peptides studied in RP-HPLC (TABLE 1), the closeness led to a recognition that the server accuracy. Next, peptides were synthesized and tested against the Gram-negative bacteria Escherichia coli and Pseudomonas aeruginosa as well as against the Gram-positive bacterium Staphylococcus aureus. Slightly different results were obtained from those reported by Souza et al. who described activity against Gram-positive bacteria but poor activity against Gram-negative species (Souza B. M. et al., Peptides 26, 2157-2164 (2005)). The chemically synthesized wild-type peptide was active against E. coli [minimal inhibitory concentration (MIC)=8.0 [symbol] mol L⁻¹] and presented the same activity against S. aureus and both of the P. aeruginosa strains tested (MIC=64.0 [symbol] mol L⁻¹—FIG. 2B). MIC results were confirmed by colony-forming unit (CFU) counts of bacteria after one day of exposure to the peptides (FIGS. 10A-10B).

Substitution analysis with Ala revealed that when Ile at position 5 and, independently, Lys at position 10 were substituted, the most drastic decreases in antimicrobial activity were observed against both Gram-positive and Gram-negative bacteria (FIG. 2B), indicating that residues [Ile]⁵ and [Lys]¹⁰ are important determinants for the biological activity of these peptides. Conversely, the single Ala-substitutions of [Gly]⁷ and [Ser]¹¹ residues led to a pronounced enhancement in antimicrobial activity (FIG. 2B). These assays further enabled the identification of a functional hotspot range determined by hydrophobicity and hydrophobic moment values for optimal antimicrobial activity of the Pol-CP—NH₂ variants (FIGS. 2C-2D). In addition, modifications to the hydrophobic face of the wild-type peptide (FIG. 2E) led to decreased antimicrobial function, with the exception of [Leu]⁶, which is at the interface between the hydrophobic and hydrophilic faces of the helical wheel and one helical step from the charged residue [Lys]¹⁰ (FIG. 2B), what may lead to destabilization of the helix, and probably, did not affect the antimicrobial activity by not changing the amphipathic balance abruptly. On the other hand, all changes made to the hydrophilic face led to increased antimicrobial activity except when the positively charged residue [Lys]¹⁰ was substituted (FIG. 2B).

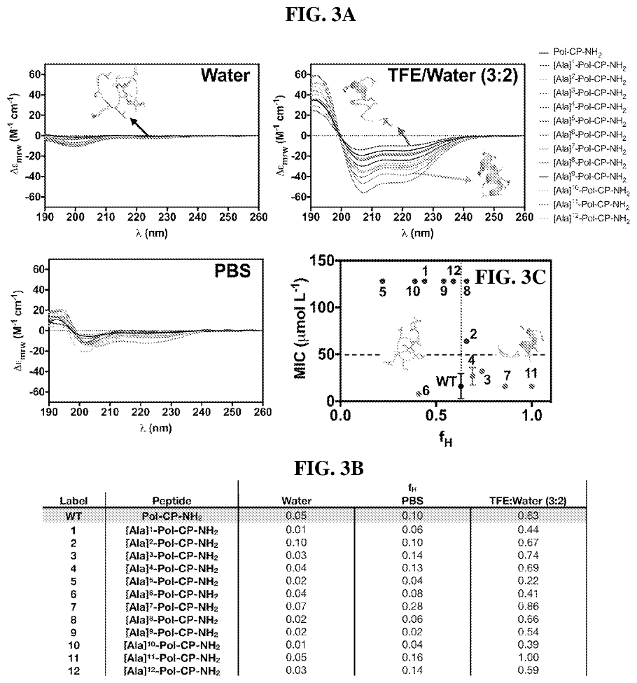

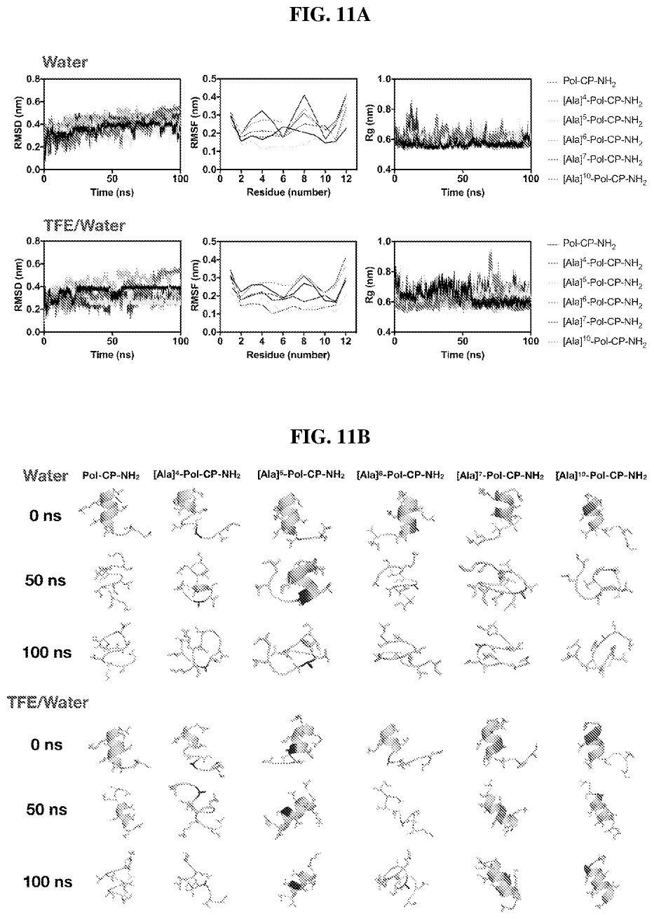

To further investigate the effect of side chains on the structure of Pol-CP—NH₂, circular dichroism (CD) spectroscopy measurements were performed, a rapid and widely used technique for analyzing peptides secondary structure, which is determinant for AMPs activity (Greenfield N. J. Trends Anal. Chem. 18, 236-244 (1999)). Among all the features that can be extracted from CD analyses, of particular interest were potential structure transitions, more specifically helix-coil transition usually observed from water or polar media to hydrophobic or helical inducer-media (Lifson S. and Roig A. J. Chem. Phys. 34, 1963-1974 (1961)), a very well-known characteristic of AMPs (Pedron C. N. et al., Eur. J. Med. Chem. 126, 456-463 (2017); Tones M. D. T. et al., ChemistrySelect 2, 18-23 (2017); Tones M. D. T. et al., J. Pept. Sci. 23, 818-823 (2017); Zelezetsky I. and Tossi A. Biochim. Biophys. Acta—Biomembr. 1758, 1436-1449 (2006); Porto W. F. et al., Nat. Commun. 9, 1490 (2018)). For this reason, the experiments were performed initially in three conditions [i.e., water, PBS buffer (pH 7.4), and trifluoroethanol (TFE) in water (3:2; v:v)] using the Ala-scan derivatives. The PBS buffer was chosen to check the effects of peptides exposure to ions at neutral pH (7.4), besides it low absorbance at the wavelength range analyzed (195 to 260 nm). TFE/water solution is widely used in studies of peptide structure as it promotes the formation of helical structures and stability (Buck M. Q. Rev. Biophys. 31, 297-355 (1998); Luo P. and Baldwin R. L. Biochemistry 36, 8413-8421 (1997)). As expected, the peptides presented an undefined secondary structure in water and a secondary structure with small helical fractions in PBS buffer (saline environment). In contrast, in the presence of TFE/water solution, the peptides tended to display a helical structure (FIG. 3A and FIGS. 11A-11B), a behavior expected for small cationic amphipathic peptides (Luo P. and Baldwin R. L. Biochemistry 36, 8413-8421 (1997)) and consistent with Lifson-Roig's helix-coil transition theory (Lifson S. and Roig A. J. Chem. Phys. 34, 1963-1974 (1961)). Most of the derivatives that presented a higher helical fraction than the wild-type (FIG. 3B) tended to be more active than the wild-type molecule against both Gram-positive and Gram-negative bacteria (FIG. 2B). Thus, the results of the present investigation reveal some correlation between the structural (FIG. 3C) and physicochemical features (FIGS. 2C-2D) with antimicrobial activity, thereby opening the door to rational design strategies. The exception was [Ala]⁶-Pol-CP—NH₂, in which the Ala-substitution led to a lower helical fraction of the peptide in helical inducer medium, and preserved the antimicrobial activity of the peptide. This might be explained by the higher helical propensity of the Leu residue when compared to the Ala residue (Pace C. N. and Scholtz J. M. Biophys. J. 75,422-427 (1998)), besides of the position of this residue in the helical wheel projection at the interface of the hydrophobic and hydrophilic faces what did not compromise the disposition of the other residues maintaining the activity of this peptide. In order to test this possibility, novel Pol-CP—NH₂ analogs were generated to further validate the optimal functional hotspot ranges observed (FIGS. 2C-2D).

Molecular dynamics (MD) simulations of the peptides were performed in water and in 60% TFE/water solution (v:v). The simulations were performed to better understand the behavior of the three-dimensional theoretical structure (FIGS. 11A-11B) of some of the Ala-scan analogs that presented different antimicrobial activities (FIGS. 2A-2E) and structural tendencies (FIGS. 3A-3C). After 100 ns of MD simulations in both media (FIG. 11B), all analogs were found to be highly stable, as indicated by the low values of root mean square deviation (RMSD), which is the measure of the average distance between the atoms of the superimposed peptides during the simulation time (Lindahl E. et al., Mol. Model. Annu. 7, 306-317 (2001); Abraham M. J. et al., SoftwareX 1-2,19-25 (2015)), and root mean square fluctuation (RMSF) obtained (FIG. 11A), which is a measure of the deviation of the position of a particle with respect to a reference position over the simulation time (Lindahl E. et al., Mol. Model. Annu. 7,306-317 (2001); Abraham M. J. et al., SoftwareX 1-2,19-25 (2015)). In water, all the peptides were mostly unstructured after 100 ns, while in the TFE/water solution [Ala]⁷-Pol-CP—NH₂ and [Ala]¹⁰-Pol-CP—NH₂ tended to display a well-defined helical structure, and [Ala]⁵-Pol-CP—NH₂ exhibited a less-defined helical structure. In addition, the radius of gyration (Rg) was maintained over time (FIG. 11A), indicating that the molecules did not bend in both media remaining helical or coiled. These parameters, in addition to the three-dimensional structures observed throughout the simulation (FIG. 11B), revealed that when substitutions are made to the hydrophilic face of Pol-CP—NH₂, the analogs appear to be less highly structured (i.e., random-coiled) in water, but helical in TFE/water solution. When changes were made to the hydrophobic core of the molecule, the tendency towards adopting a helical structure was maintained in TFE/water and sometimes decreased in the same medium (FIG. 11B), consistent with the CD spectra results (FIG. 3A). Samples in TFE/water had similar RMSD, RMSF, and Rg values (FIG. 11A) in comparison with simulations in water alone, indicating the structural stability of this family of peptides (FIG. 11B).

TABLE 1
Summary of Pol-CP-NH₂ and designed analogs.

| | | | Molecular | Observed | |
|---|---|---|---|---|---|
| | | | Weight | Molecular | Purity |
| Label | Peptide | Sequence | (Da) | Weight (Da)^{a} | (%)^{b} |
| WT | Pol-CP-NH₂ | ILGTILGLLKSL-NH₂ | 1239.8 | 1240.9 | 99 |
| 1 | [Ala]¹-Pol-CP-NH₂ | ALGTILGLLKSL-NH₂ | 1197.8 | 1198.7 | 95 |
| 2 | [Ala]²-Pol-CP-NH₂ | IAGTILGLLKSL-NH₂ | 1197.8 | 1198.8 | 96 |
| 3 | [Ala]³-Pol-CP-NH₂ | ILATILGLLKSL-NH₂ | 1253.8 | 1254.8 | 96 |
| 4 | [Ala]⁴-Pol-CP-NH₂ | ILGAILGLLKSL-NH₂ | 1209.8 | 1210.8 | 93 |
| 5 | [Ala]⁵-Pol-CP-NH₂ | ILGTALGLLKSL-NH₂ | 1197.8 | 1198.8 | 94 |
| 6 | [Ala]⁶-Pol-CP-NH₂ | ILGTIAGLLKSL-NH₂ | 1197.8 | 1198.8 | 93 |
| 7 | [Ala]⁷-Pol-CP-NH₂ | ILGTILALLKSL-NH₂ | 1253.8 | 1254.8 | 92 |
| 8 | [Ala]⁸-Pol-CP-NH₂ | ILGTILGALKSL-NH₂ | 1197.8 | 1198.8 | 93 |
| 9 | [Ala]⁹-Pol-CP-NH₂ | ILGTILGLAKSL-NH₂ | 1197.8 | 1198.8 | 90 |
| 10 | [Ala]¹⁰-Pol-CP-NH₂ | ILGTILGLLASL-NH₂ | 1182.8 | 1183.8 | 92 |
| 11 | [Ala]¹¹-Pol-CP-NH₂ | ILGTILGLLKAL-NH₂ | 1223.8 | 1224.7 | 95 |
| 12 | [Ala]¹²-Pol-CP-NH₂ | ILGTILGLLKSA-NH₂ | 1197.8 | 1198.8 | 92 |
| 13 | [Leu]⁵-[Lys]⁹-Pol- | ILGTLLGLKKSL-NH₂ | 1254.8 | 1256.0 | 99 |
| | CP-NH₂ | | | | |
| 14 | [Lys]⁵-Pol-CP-NH₂ | ILGTKLGLLKSL-NH₂ | 1254.8 | 1255.9 | 99 |
| 15 | [Lys]⁴-Pol-CP-NH₂ | ILGKILGLLKSL-NH₂ | 1265.8 | 1266.8 | 98 |
| 16 | [Lys]⁷-Pol-CP-NH₂ | ILGTILKLLKSL-NH₂ | 1309.8 | 1310.9 | 99 |
| 17 | [Phe]⁹-Pol-CP-NH₂ | ILGTILGLFKSL-NH₂ | 1272.8 | 1274.0 | 99 |
| 18 | Des[Leu]¹²-Pol-CP- | ILGTILGLLKSL-NH₂ | 1125.8 | 1126.8 | 99 |
| | NH₂ | | | | |
| 19 | [Glu]³-[Lys]⁵- | ILETKLGLLKSE-NH₂ | 1341.8 | 1341.8 | 99 |
| | [Glu]¹²-Pol-CP-NH₂ | | | | |
| 20 | [Gly]¹-Pol-CP-NH₂ | GLGTILGLLKSL-NH₂ | 1182.8 | 1183.8 | 99 |

| | HPLC | | | | | |
|---|---|---|---|---|---|---|
| | Retention | HC₅₀ | MIC Average | | Cytotoxicity | SEQ ID |
| Label | Time (min)^{c} | ( [symbol] mol L⁻¹)^{d} | ( [symbol] mol L⁻¹) | SI^{e} | ( [symbol] mol L⁻¹) | NO: |
| WT | 16.5 | 50.0 | 16.2 | 3.1 | 32.0 | 1 |
| 1 | 15.8 | — | — | — | — | 2 |
| 2 | 15.4 | — | — | — | — | 3 |
| 3 | 16.8 | — | — | — | >64.0 | 4 |
| 4 | 16.8 | — | — | — | — | 5 |
| 5 | 14.4 | — | — | — | >64.0 | 6 |
| 6 | 15.0 | — | — | — | — | 7 |
| 7 | 16.8 | — | — | — | 32.0 | 8 |
| 8 | 15.0 | — | — | — | — | 9 |
| 9 | 14.5 | — | — | — | — | 10 |
| 10 | 17.5 | — | — | — | — | 11 |
| 11 | 16.7 | — | — | — | 64.0 | 12 |
| 12 | 14.6 | — | — | — | — | 13 |
| 13 | 11.5 | >100.0 | >50.0 | — | — | 14 |
| 14 | 11.5 | >100.0 | >50.0 | — | — | 15 |
| 15 | 15.0 | >100.0 | 3.3 | — | 32.0 | 16 |
| 16 | 16.0 | 12.5 | 1.4 | 9.2 | 16.0 | 17 |
| 17 | 15.7 | 50.0 | 20.0 | 2.5 | — | 18 |
| 18 | 13.4 | >100.0 | >50.0 | — | — | 19 |
| 19 | 9.2 | >100.0 | >50.0 | — | — | 20 |
| 20 | 15.5 | >100.0 | 16.7 | — | >64.0 | 21 |

^{a}LC/ESI-MS data were obtained on a Model 6130 Infinity mass spectrometer coupled to a Model 1260 HPLC system (Agilent), using a Phenomenex Gemini C18 column (2.0 mm × 150 mm, 3.0 μm particles, 110 Å pores). Solvent A was 0.1% TFA in water, and solvent B was 90% ACN in solvent A. Elution with a 5-95% B gradient was performed over 20 min, 0.2 mL min−1 flow and peptides were detected at 220 nm. Mass measurements were performed in a positive mode with the following conditions: mass range between 100 to 2500 m/z, ion energy of 5.0 V, nitrogen gas flow of 12 L min⁻¹, solvent heater of 250° C., multiplier of 1.0, capillary of 3.0 kV and cone voltage of 35 V.
^{b, c}HPLC profiles were obtained under the following conditions: Column Supelcosil C18 (4.6 × 150 mm), 60 Å, 5 μm; Solvent System: A (0.1% TFA/H₂O) and B (0.1% TFA in 90% ACN/H₂O); Gradient: 5-95% B in 30 minutes; Flow: 1.0 mL min⁻¹; λ = 220 nm; Injection Volume: 50 μL and Sample Concentration: 1.0 mg mL⁻¹.
^{d}Concentration needed for 50% hemolysis caused by RBC exposure to peptides.
^{e}Selectivity Index = HC₅₀/MIC_{average} indicating peptides selectivity when in the presence of human erythrocytes.

### EXAMPLE 2

### Rationally Designed Pol-CP—NH₂ Derivatives

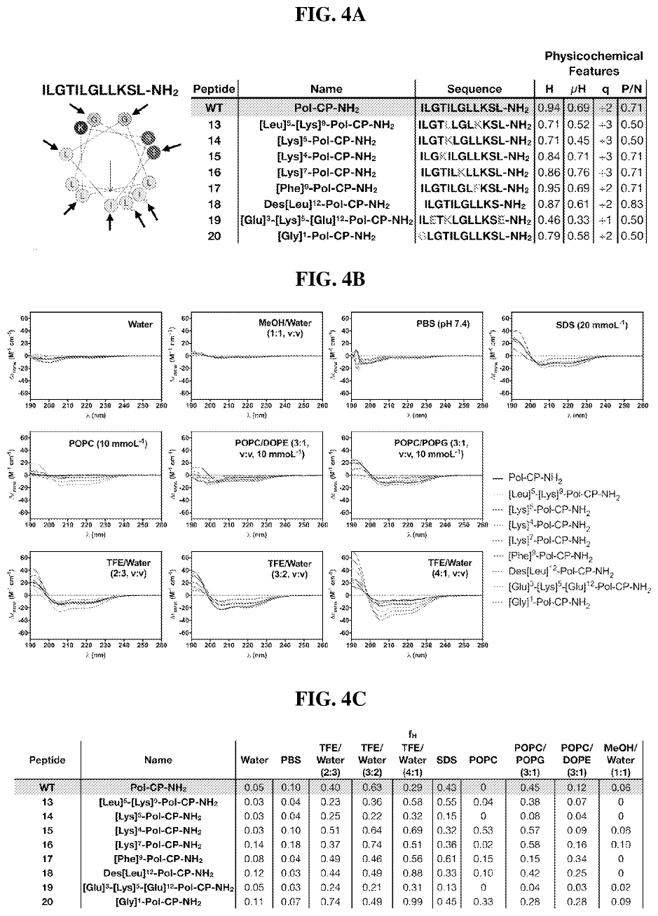

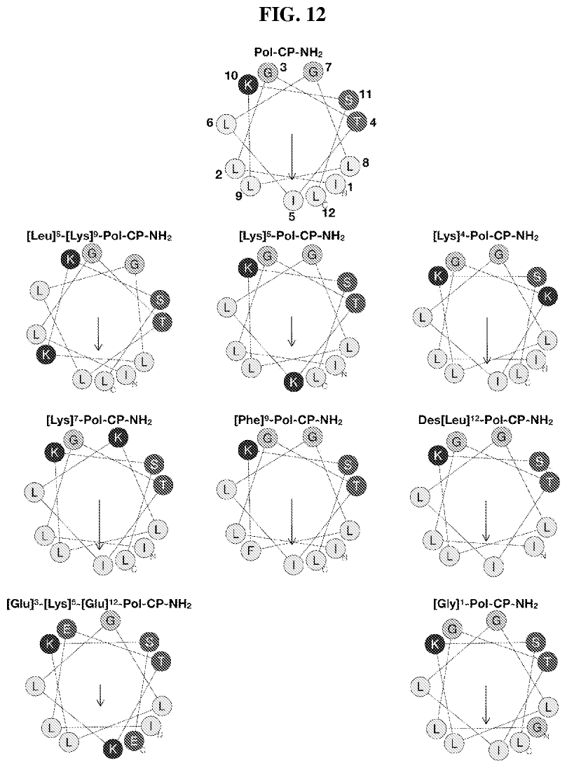

Most wasp venom peptides present conserved motifs in their sequences, e.g., Pol-CP—NH₂ is similar to protonectin (Ile-Leu-Gly-Thr-Ile-Leu-Gly-Leu-Leu-Lys-Gly-Leu-NH₂ (SEQ ID NO: 385)) (Mendes M. A. et al., Toxicon 44,67-74 (2004)). Therefore, to design the next generation of Pol-CP—NH₂ derivatives, single-substitution mutants were generated to elucidate structure-function relationships and to identify physicochemical activity determinants (FIG. 4A and FIG. 12). The positions selected for the substitutions were chosen based on the Ala-scan screening results obtained (FIGS. 2A-2E), and modifications were rationally proposed by fine-tuning select physicochemical functional determinants (i.e., hydrophobicity, hydrophobic moment, and helical propensity).

To introduce charge into the sequence (Pace C. N. and Scholtz J. M. Biophys. J. 75, 422-427 (1998)), Lys was used rather than Arg due to its superior flexibility, lower propensity in potentially toxic cell penetrating peptides (Cutrona K. J. et al., FEBS Lett. 589, 3915-3920 (2015)), and decreased hydrophobic side chain, which is associated with cytotoxicity (Eisenberg D. Ann. Rev. Biochem. 53,595-623 (1984)). Moreover, Lys residues are more frequent than Arg residues in naturally occurring wasp venom peptides (Lee S. H. et al., Toxins (Basel). 8, 1-29 (2016)).

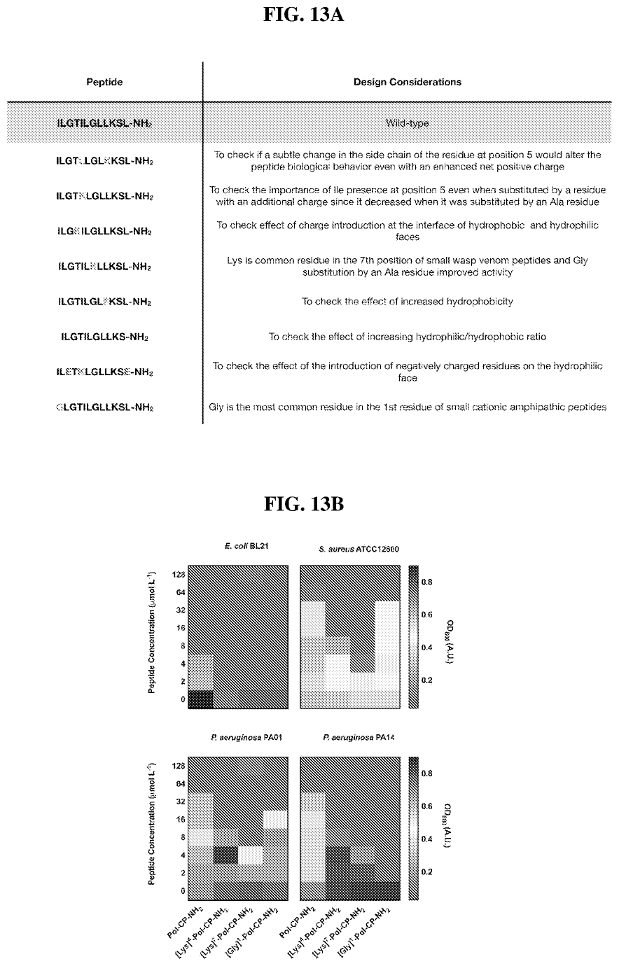

Hydrophobicity was incorporated into the sequence via substitution of residues from the wild-type sequence by Leu and Phe. Leu was chosen because a minimal amount of energy is required for it to adopt a helical structure (Pace C. N. and Scholtz J. M. Biophys. J. 75,422-427 (1998)), which favors antimicrobial activity (FIGS. 1A-1C and FIGS. 2A-2E), and it occurs at high frequency in wasp venom peptide sequences (Lee S. H. et al., Toxins (Basel). 8,1-29 (2016)). On the other hand, Phe was chosen due to its bulky effect and higher hydrophobicity values (Eisenberg D. Ann. Rev. Biochem. 53,595-623 (1984)), making it possible to evaluate the effect of adding an aromatic residue to the hydrophobic face on structure and biological function. Additionally, unlike Trp, Phe residues are not major components of cell-penetrating peptides (Jin L. et al., J. Med. Chem. 59,1791-1799 (2016)), which are typically cytotoxic, and are therefore better candidates for peptide design. Taking these guidelines into account (FIGS. 13A-13B), a second-generation peptide library was generated that aimed to unveil further structure-activity relationships (SAR) (FIG. 4A).

First, the effects of each substitution on the theoretical values specific physicochemical features was assessed (FIG. 4A) and the structures of these new analogs were analyzed by circular dichroism (CD) in ten different media (FIG. 4B) that mimicked potential environments encountered by peptides, such as water, saline, and hydrophobic environments. Bacterial membranes are composed of anionic lipids, such as phosphatidylglycerol (PG), and zwitterionic lipids, such as phosphatidylethanolamine (PE), which are important for membrane organization. The lipid composition varies among bacteria, e.g., the cell membrane of Gram-negative bacteria presents a higher content of PE than that of Gram-positives; on the other hand, Gram-positive membranes are composed of higher levels of anionic lipids (e.g., PG) (Epand R. M. and Epand R. F. Biochim. Biophys. Acta—Biomembr. 1788,289-294 (2009)). In order to mimic these membrane environments (Chongsiriwatana N. P. and Barron A. E. Humana Press. 171-182 (2010); De Kruijff B. et al., Academic Press. 44,477-515 (1997); Chou H. T. et al., Peptides 31,1811-1820 (2010)), one micelle and three vesicle formulations were prepared: SDS (20 mmol L⁻¹), POPC (10 mmol L⁻¹), and POPC:DOPE (3:1, mol:mol, 10 mmol L⁻¹), zwitterionic lipids, and POPC:POPG (3:1, mol:mol, 10 mmol L⁻¹), a negatively charged unilamellar vesicle. The structure of the peptides in TFE/water solutions, which are well known peptide helix inducers, was also analyzed (Luo P. and Baldwin R. L. Biochemistry 36,8413-8421 (1997)). The helical fraction values obtained in all CD spectra analyses are shown in FIG. 4C and the most active peptides are inside the hotspot predicted with the Ala-Scan analogs previously. Pol-CP—NH₂ and analogs did not tend to ®-conformations in the presence of MeOH, which is known as a ®-structure promoter (Radhakrishnan M. et al., ChemBioChem 6,2152-2158 (2005)). Peptides presented higher helical fraction values when in contact with negatively charged and zwitterionic vesicles than when in contact to positively charged vesicles. The exceptions were the most hydrophobic analog, [Phe]⁹-Pol-CP—NH₂, and [Gly]¹-Pol-CP—NH₂ that presented the same helical fraction values in contact with negatively and positively charged vesicles, interestingly this peptide presented high helical fraction values when in contact with zwitterionic vesicles even with the introduction of a Gly residue that does not show high helical propensity. The antimicrobial activity of [Gly]¹-Pol-CP—NH₂ was similar to the most active analogs with higher positive net charge as can be observe in FIG. 4C.

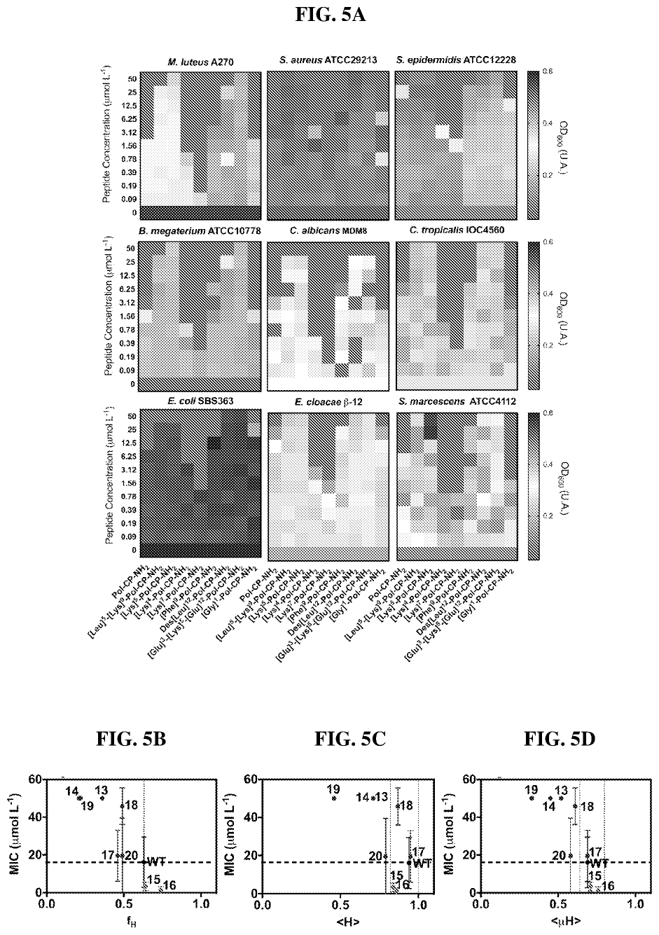

Next, peptides were tested against a larger panel of Gram-positive and Gram-negative bacteria and two species of Candida (FIG. 5A). As anticipated by the previous structure-activity relationship (SAR) analysis (FIGS. 2A-2E, FIGS. 3A-3C, and FIGS. 4A-4C), mutations made within the hydrophobic face led to decreased helical fraction values (FIG. 5B) and resulted in loss of antimicrobial activity (FIG. 5A). The hydrophobicity and hydrophobic moment functional hotpots identified previously (FIGS. 2A-2E) also correlated here with maximal antimicrobial activity in the nanomolar range (FIGS. 5B-5D), showing that these features affect the optimal conditions of this family of peptides leading to higher antimicrobial activity when in its optimal range. This behavior confirms the importance of the hydrophobic face of the peptide in both structure and activity, since one can observe clearly a helix-coil transition when peptides are in contact with membranes or membrane-like environments, such as the vesicles used in the CD experiments. The three most active AMPs, [Lys]⁴-Pol-CP—NH₂, [Lys]⁷-Pol-CP—NH₂ and [Gly]¹-Pol-CP—NH₂, were tested against the initial panel of bacteria (E. coli BL21, P. aeruginosa PA01 and PA14, and S. aureus ATCC12600—FIGS. 13A). All peptides were active against E. coli, even at very low concentrations (<2 [symbol] mol L⁻¹), and moderately active against P. aeruginosa PA01 (8-32 [symbol] mol L⁻¹), with [Gly]¹-Pol-CP—NH₂ presenting surprisingly high activity against P. aeruginosa PA14 (<2 [symbol] mol L⁻¹) (FIG. 13B). The peptides, except for [Gly]¹-Pol-CP—NH₂ (64 [symbol] mol L⁻¹), showed high activity against S. aureus (8-16 [symbol] mol L⁻¹) (FIG. 13B). Thus, synthetic peptides exhibited differential antimicrobial activity, which was predicted by physicochemical parameters.

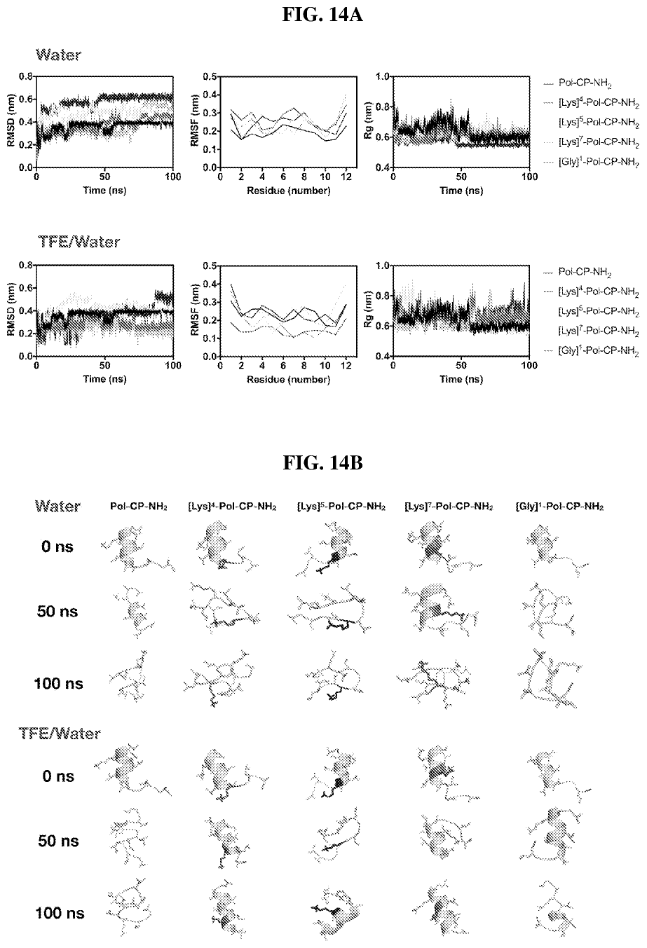

MD simulations were performed in water and 60% TFE/water solution (v:v) (FIGS. 14A-14B) for the three most active peptides ([Lys]⁴-Pol-CP—NH₂, [Lys]⁷-Pol-CP—NH₂, and [Gly]¹-Pol-CP—NH₂) (FIG. 5A) from the second generation library (FIG. 4A) and one of the least active analogs ([Lys]⁵-Pol-CP—NH₂) (FIG. 5A). The simulations showed that the peptides were less highly structured in water than in the TFE/water solution. Differently from the Ala-scan results, in TFE/water medium, the introduction of a Lys residue in the hydrophobic face core ([Lys]⁵-Pol-CP—NH₂) preserved the peptide structure (FIG. 14B), probably because of hydrophobic interactions provided by the longer aliphatic portion of the Lys side chain compared to the Ala side chain previously introduced. Substitutions made to the hydrophilic face led to stabilized helical structures (FIG. 14A), with increased helical content when compared to the wild-type peptide (FIG. 4C and FIG. 14B). On the other hand, the introduction of a Gly residue to the hydrophobic face of the peptide destabilized the N-terminus of the structure (FIG. 14A), as expected: Gly is known to increase flexibility and disfavor helical structure (Zelezetsky I. and Tossi A. Biochim. Biophys. Acta—Biomembr. 1758, 1436-1449 (2006)) and is generally directly correlated with increased cytotoxic activity (Pacor S. et al., J. Antimicrob. Chemother. 50, 339-348 (2002)).

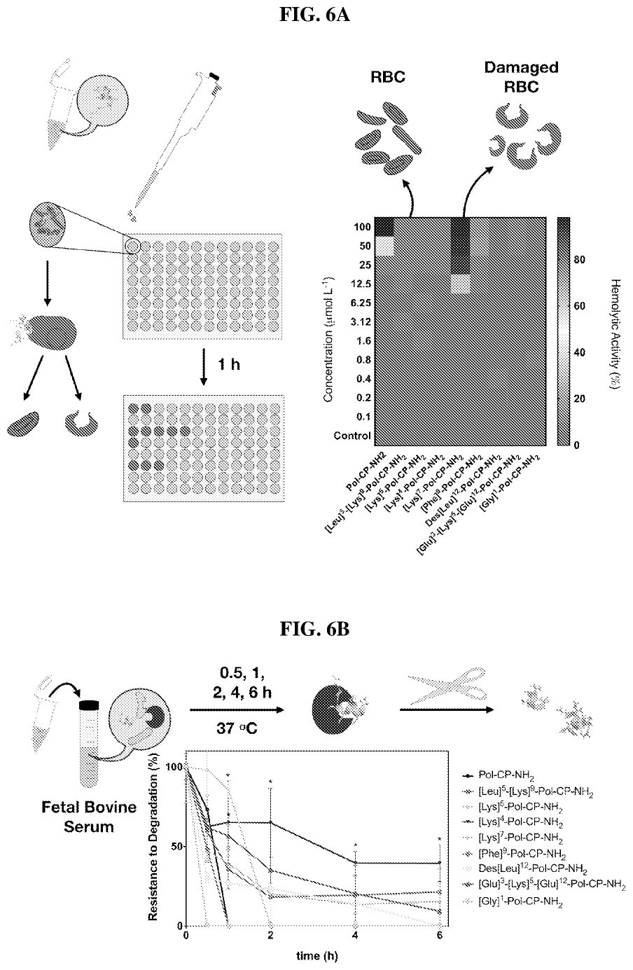

The hemolytic activity of AMPs directly correlates with their interaction with zwitterionic membranes, which they subsequently lyse (Jin Y. et al., ntimicrob. Agents Chemother. 49, 4957-4964 (2005)). Tuning AMP features to modulate membrane interactions to minimize their effect on erythrocyte membranes while preserving activity against bacteria is a long-standing goal in the field. One of the most important parameters to achieve this selectivity is tuning the electronic density—positively charged surfaces—of AMPs, which are attracted to the negatively charged membranes of microorganisms, whereas eukaryotic cells display zwitterionic lipids in their membrane (Lohner K. Norfolk: Horizon Scientific Press. 149-165 (2001)). Mammalian cells present higher amounts of cholesterol in their membrane, which stabilizes the lipid bilayer by increasing cohesion and mechanical stiffness (Henriksen J. et al., Biophys. J. 90, 1639-1649 (2006)), making it difficult for the membranes to bend and, consequently, to be permeabilized by AMPs. After the initial electrostatic interactions, the hydrophobic face of the amphipathic structure of AMPs interacts directly with the nonpolar region of the microorganism membrane, destabilizing it and leading to membrane disruption and cell death (Nagaraj N. S. Curr. Pharm. Des. 8, 727-742 (2002); Yeaman M. R. and Yount N. Y. Pharmacol. Rev. 55, 27 LP-55 (2003)). The design methodology focused primarily in enhancing features that would increase peptides interaction with negatively charged membranes. Thus, the hemolytic activity (FIG. 6A) of the peptides was tested to check their translatability prior to in vivo assays. Analog [Phe]⁹-Pol-CP—NH₂ was as hemolytic as the wild-type peptide (between 50-100 μmol L⁻¹). [Lys]⁷-Pol-CP—NH₂ was the only analog with higher hemolytic activity than the wild-type (12.5 [symbol] mol L⁻¹). None of the other analogs exhibited hemolytic activity in the range of concentrations evaluated (0-100 [symbol] mol L⁻¹).

Stability is an issue often limiting the translation of AMPs into the clinic (Seo M. D. et al., Molecules 17, 12276-12286 (2012)). Pol-CP—NH₂ is a natural occurring cationic AMP, and most cationic peptides are not stable in the presence of peptidases (Diao L. and Meibohm B. Clin. Pharmacokinet. 52, 855-868 (2013)). The stability of the second generation of Pol-CP—NH₂ derivatives (FIG. 4A) in fetal bovine serum was assessed (FIG. 6B). Most analogs were degraded in a few minutes after exposure to serum proteases, including [Gly]¹-Pol-CP—NH₂. However, [Lys]'-Pol-CP—NH₂ and [Lys]⁴-Pol-CP—NH₂ demonstrated increased resistance to protease-mediated degradation, particularly [Lys]⁴-Pol-CP—NH₂, which persisted (˜50% of initial concentration added) even after six hours of exposure (FIG. 6B). The introduction of Lys residues in both cases favored a higher helical stabilization compared to the other modifications made (FIGS. 4B-4C) and this is known as strategy to achieve higher resistance to degradation (Villegas V. et al., Fold. Des. 1, 29-34 (1996)). However, there are other elaborated approaches that could be used as potential stability enhancers, such as introducing restrictions (lactam and disulfide bridged peptides), cyclic peptides and/or introduction of lipids or carbohydrates as peptides conjugates (van Witteloostuijin S. B. et al., ChemMedChem 11, 2474-2495 (2016)).

### EXAMPLE 3

### Cytotoxicity Against Mammalian Cells and In Vivo Antimicrobial Activity Against P. aeruginosa

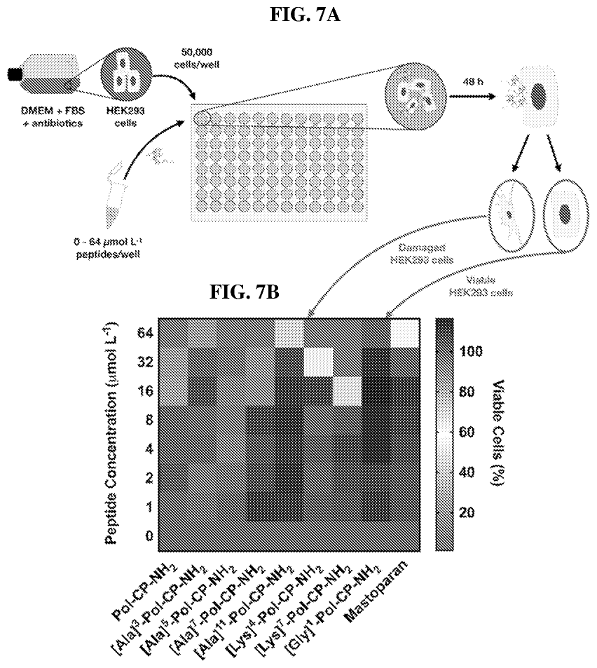

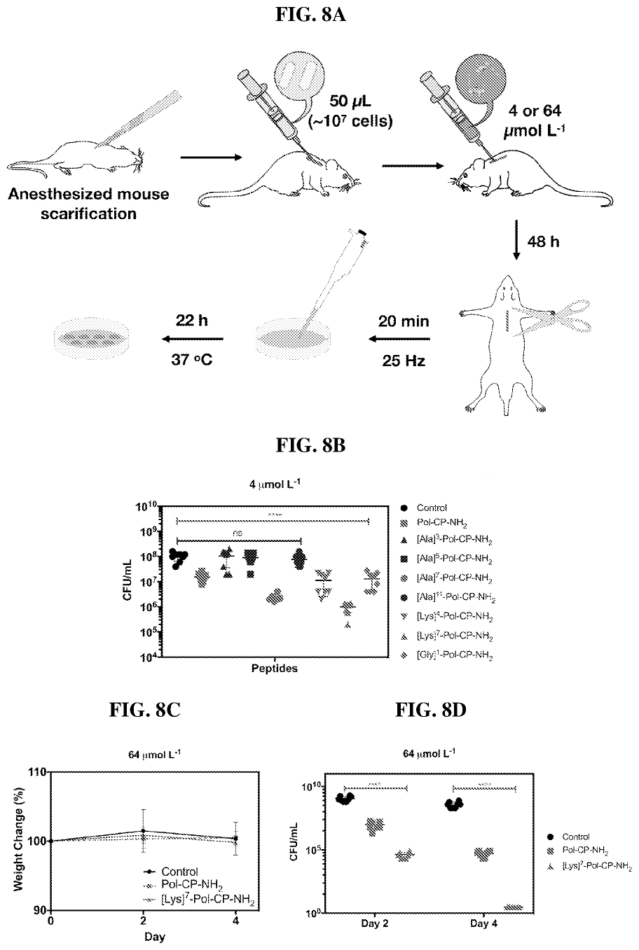

Several peptides from both generations identified as most active (i.e., antimicrobial hits) and least active (i.e., negative controls) against the Gram-negative bacterium P. aeruginosa (FIGS. 2A-2E and FIGS. 4A-4C) were tested for cytotoxicity against human embryonic kidney cells (HEK293) (FIGS. 7A-7B). The wild-type peptide presented cytotoxic activity at a lower concentration (32 μmol L⁻¹) than its MIC against P. aeruginosa (64 μmol L⁻¹), whereas all synthetic analogs presented low cytotoxicity against HEK293 cells (FIG. 7B). The lead peptides ([Lys]⁴-Pol-CP—NH₂ and [Lys]'-Pol-CP—NH₂) exhibited certain cytotoxicity at concentrations two- to four-fold higher than their MICs against P. aeruginosa (FIG. 7B). The least active analogs were not cytotoxic in the range analyzed (0-64 μmol L⁻¹) (FIG. 7B). The lead peptide hit, [Lys]⁷-Pol-CP—NH₂, displayed cytotoxicity at 16 μmol L⁻¹; therefore, a nontoxic dose (4 μmol L⁻¹) of this and the other lead peptides was used to assess their anti-infective potential in vivo using a scarification mouse model (FIGS. 8A-8B).

A skin abscess was induced in mice, after which a single dose of 4 μmol L⁻¹ of peptides was administered (FIG. 8A). The antimicrobial activity of all peptides was consistent with results obtained in vitro (FIGS. 2A-2E and FIGS. 5A-5D). The lead peptide derivatives, having substitutions in position 7 ([Ala]⁷-Pol-CP—NH₂ and [Lys]⁷-Pol-CP—NH₂), were the most active, and [Gly]¹-Pol-CP—NH₂ and [Lys]⁴-Pol-CP—NH₂ demonstrated comparable activity to the wild-type peptide (FIG. 8B). A single dose of the lead peptide [Lys]⁷-Pol-CP—NH₂, which was non-toxic to mice (Aston W. J. et al., BMC Cancer 17, 684 (2017); Zhang Q. et al., Toxicol. Reports 2, 546-554 (2015); Lobo E. D. and Balthasar J. P. J. Pharm. Sci. 92, 1654-1664 (2003); Hassan F. et al., PLoS One 13, e0192882 (2018)) (FIG. 8C), further demonstrated anti-infective activity virtually sterilizing abscess infections after 4 days (FIG. 8D).

### EXAMPLE 4

### Additional Peptides of Interest

Additional K7-Polybia-CP—NH₂ variants were identified for future analysis using ILGTILKLLKSL (SEQ ID NO: 17) as template, as well as L10-Decoralin-NH2 variants using SLLSLIRKLLT (SEQ ID NO: 22) as template (TABLE 2). For K7-Polybia-CP—NH₂ variants, changes at positions 5 (i.e., [Ile]⁵) and 7 (i.e., [Lys]⁷) result in sharp drops in activity, and for L10-Decoralin-NH₂ variants, changes at positions 8 (i.e., [Lys]⁸) and 10 (i.e., [Leu]¹⁰) result in sharp drops in activity. D-amino analogs and cyclic analogs (synthesized containing the restriction in positions 0 and 13 (CILGTILKLLKSLC; SEQ ID NO: 384) for K7-Polybia-CP—NH₂ variants and positions 0 and 12 (CSLLSLIRKLLTC; SEQ ID NO: 385) for L10-Decoralin-NH₂ variants) will be explored as well.

TABLE 2
Additional AMP of interest.

| | | SEQ |
|---|---|---|
| | | ID |
| Peptide | Sequence | NO: |
| K7-Polybia-CP-NH2 | ALGTILKLLKSL | 23 |
| K7-Polybia-CP-NH2 | CLGTILKLLKSL | 24 |
| K7-Polybia-CP-NH2 | DLGTILKLLKSL | 25 |
| K7-Polybia-CP-NH2 | ELGTILKLLKSL | 26 |
| K7-Polybia-CP-NH2 | FLGTILKLLKSL | 27 |
| K7-Polybia-CP-NH2 | GLGTILKLLKSL | 28 |
| K7-Polybia-CP-NH2 | HLGTILKLLKSL | 29 |
| K7-Polybia-CP-NH2 | KLGTILKLLKSL | 30 |
| K7-Polybia-CP-NH2 | LLGTILKLLKSL | 31 |
| K7-Polybia-CP-NH2 | MLGTILKLLKSL | 32 |
| K7-Polybia-CP-NH2 | NLGTILKLLKSL | 33 |
| K7-Polybia-CP-NH2 | PLGTILKLLKSL | 34 |
| K7-Polybia-CP-NH2 | QLGTILKLLKSL | 35 |
| K7-Polybia-CP-NH2 | RLGTILKLLKSL | 36 |
| K7-Polybia-CP-NH2 | SLGTILKLLKSL | 37 |
| K7-Polybia-CP-NH2 | TLGTILKLLKSL | 38 |
| K7-Polybia-CP-NH2 | VLGTILKLLKSL | 39 |
| K7-Polybia-CP-NH2 | WLGTILKLLKSL | 40 |
| K7-Polybia-CP-NH2 | YLGTILKLLKSL | 41 |
| K7-Polybia-CP-NH2 | IAGTILKLLKSL | 42 |
| K7-Polybia-CP-NH2 | ICGTILKLLKSL | 43 |
| K7-Polybia-CP-NH2 | IDGTILKLLKSL | 44 |
| K7-Polybia-CP-NH2 | IEGTILKLLKSL | 45 |
| K7-Polybia-CP-NH2 | IKGTILKLLKSL | 46 |
| K7-Polybia-CP-NH2 | TGGTILKLLKSL | 47 |
| K7-Polybia-CP-NH2 | IHGTILKLLKSL | 48 |
| K7-Polybia-CP-NH2 | IIGTILKLLKSL | 49 |
| K7-Polybia-CP-NH2 | IKGTILKLLKSL | 50 |
| K7-Polybia-CP-NH2 | IMGTILKLLKSL | 51 |
| K7-Polybia-CP-NH2 | INGTILKLLKSL | 52 |
| K7-Polybia-CP-NH2 | IPGTILKLLKSL | 53 |
| K7-Polybia-CP-NH2 | IQGTILKLLKSL | 54 |
| K7-Polybia-CP-NH2 | IRGTILKLLKSL | 55 |
| K7-Polybia-CP-NH2 | ISGTILKLLKSL | 56 |
| K7-Polybia-CP-NH2 | IYGTILKLLKSL | 57 |
| K7-Polybia-CP-NH2 | IVGTILKLLKSL | 58 |
| K7-Polybia-CP-NH2 | IWGTILKLLKSL | 59 |
| K7-Polybia-CP-NH2 | IYGTILKLLKSL | 60 |
| K7-Polybia-CP-NH2 | ILATILKLLKSL | 61 |
| K7-Polybia-CP-NH2 | ILCTILKLLKSL | 62 |
| K7-Polybia-CP-NH2 | ILDTILKLLKSL | 63 |
| K7-Polybia-CP-NH2 | ILETILKLLKSL | 64 |
| K7-Polybia-CP-NH2 | ILFTILKLLKSL | 65 |
| K7-Polybia-CP-NH2 | ILHTILKLLKSL | 66 |
| K7-Polybia-CP-NH2 | ILITILKLLKSL | 67 |
| K7-Polybia-CP-NH2 | ILKTILKLLKSL | 68 |
| K7-Polybia-CP-NH2 | ILLTILKLLKSL | 69 |
| K7-Polybia-CP-NH2 | ILMTILKLLKSL | 70 |
| K7-Polybia-CP-NH2 | ILNTILKLLKSL | 71 |
| K7-Polybia-CP-NH2 | ILPTILKLLKSL | 72 |
| K7-Polybia-CP-NH2 | ILQTILKLLKSL | 73 |
| K7-Polybia-CP-NH2 | ILRTILKLLKSL | 74 |
| K7-Polybia-CP-NH2 | ILSTILKLLKSL | 75 |
| K7-Polybia-CP-NH2 | ILTTILKLLKSL | 76 |
| K7-Polybia-CP-NH2 | ILVTILKLLKSL | 77 |
| K7-Polybia-CP-NH2 | ILWTILKLLKSL | 78 |
| K7-Polybia-CP-NH2 | ILYTILKLLKSL | 79 |
| K7-Polybia-CP-NH2 | ILGAILKLLKSL | 80 |
| K7-Polybia-CP-NH2 | ILGCILKLLKSL | 81 |
| K7-Polybia-CP-NH2 | ILGDILKLLKSL | 82 |
| K7-Polybia-CP-NH2 | ILGEILKLLKSL | 83 |
| K7-Polybia-CP-NH2 | ILGPILKLLKSL | 84 |
| K7-Polybia-CP-NH2 | ILGGILKLLKSL | 85 |
| K7-Polybia-CP-NH2 | ILGHILKLLKSL | 86 |
| K7-Polybia-CP-NH2 | ILGIILKLLKSL | 87 |
| K7-Polybia-CP-NH2 | ILGKILKLLKSL | 88 |
| K7-Polybia-CP-NH2 | ILGLILKLLKSL | 89 |
| K7-Polybia-CP-NH2 | ILGMILKLLKSL | 90 |
| K7-Polybia-CP-NH2 | ILGNILKLLKSL | 91 |
| K7-Polybia-CP-NH2 | ILGPILKLLKSL | 92 |
| K7-Polybia-CP-NH2 | ILGQILKLLKSL | 93 |
| K7-Polybia-CP-NH2 | ILGRILKLLKSL | 94 |
| K7-Polybia-CP-NH2 | ILGSILKLLKSL | 95 |
| K7-Polybia-CP-NH2 | ILGVILKLLKSL | 96 |
| K7-Polybia-CP-NH2 | ILGWILKLLKSL | 97 |
| K7-Polybia-CP-NH2 | ILGYILKLLKSL | 98 |
| K7-Polybia-CP-NH2 | ILGIIAKLLKSL | 99 |
| K7-Polybia-CP-NH2 | ILGTICKLLKSL | 100 |
| K7-Polybia-CP-NH2 | ILGTIDKLLKSL | 101 |
| K7-Polybia-CP-NH2 | ILGTIEKLLKSL | 102 |
| K7-Polybia-CP-NH2 | ILGTIFKLLKSL | 103 |
| K7-Polybia-CP-NH2 | ILGTIGKLLKSL | 104 |
| K7-Polybia-CP-NH2 | ILGTIHKLLKSL | 105 |
| K7-Polybia-CP-NH2 | ILGTIIKLLKSL | 106 |
| K7-Polybia-CP-NH2 | ILGTIKKLLKSL | 107 |
| K7-Polybia-CP-NH2 | ILGTIMKLLKSL | 108 |
| K7-Polybia-CP-NH2 | ILGTINKLLKSL | 109 |
| K7-Polybia-CP-NH2 | ILGTIPKLLKSL | 110 |
| K7-Polybia-CP-NH2 | ILGTIQKLLKSL | 111 |
| K7-Polybia-CP-NH2 | ILGTIRKLLKSL | 112 |
| K7-Polybia-CP-NH2 | ILGTISKLLKSL | 113 |
| K7-Polybia-CP-NH2 | ILGTITKLLKSL | 114 |
| K7-Polybia-CP-NH2 | ILGTIVKLLKSL | 115 |
| K7-Polybia-CP-NH2 | ILGTIWKLLKSL | 116 |
| K7-Polybia-CP-NH2 | ILGTIYKLLKSL | 117 |
| K7-Polybia-CP-NH2 | ILGTILKALKSL | 118 |
| K7-Polybia-CP-NH2 | ILGTILKCLKSL | 119 |
| K7-Polybia-CP-NH2 | ILGTILKDLKSL | 120 |
| K7-Polybia-CP-NH2 | ILGTILKELKSL | 121 |
| K7-Polybia-CP-NH2 | ILGTILKFLKSL | 122 |
| K7-Polybia-CP-NH2 | ILGTILKGLKSL | 123 |
| K7-Polybia-CP-NH2 | ILGTILKHLKSL | 124 |
| K7-Polybia-CP-NH2 | ILGTILKILKSL | 125 |
| K7-Polybia-CP-NH2 | ILGTILKKLKSL | 126 |
| K7-Polybia-CP-NH2 | ILGTILKMLKSL | 127 |
| K7-Polybia-CP-NH2 | ILGTILKNLKSL | 128 |
| K7-Polybia-CP-NH2 | ILGTILKPLKSL | 129 |
| K7-Polybia-CP-NH2 | ILGTILKQLKSL | 130 |
| K7-Polybia-CP-NH2 | ILGTILKRLKSL | 131 |
| K7-Polybia-CP-NH2 | ILGTILKSLKSL | 132 |
| K7-Polybia-CP-NH2 | ILGTILKTLKSL | 133 |
| K7-Polybia-CP-NH2 | ILGTILKVLKSL | 134 |
| K7-Polybia-CP-NH2 | ILGTILKWLKSL | 135 |
| K7-Polybia-CP-NH2 | ILGTILKYLKSL | 136 |
| K7-Polybia-CP-NH2 | ILGTILKLAKSL | 137 |
| K7-Polybia-CP-NH2 | ILGTILKLCKSL | 138 |
| K7-Polybia-CP-NH2 | ILGTILKLDKSL | 139 |
| K7-Polybia-CP-NH2 | ILGTILKLEKSL | 140 |
| K7-Polybia-CP-NH2 | ILGTILKLFKSL | 141 |
| K7-Polybia-CP-NH2 | ILGTILKLGKSL | 142 |
| K7-Polybia-CP-NH2 | ILGTILKLHKSL | 143 |
| K7-Polybia-CP-NH2 | ILGTILKLIKSL | 144 |
| K7-Polybia-CP-NH2 | ILGTILKLKKSL | 145 |
| K7-Polybia-CP-NH2 | ILGTILKLMKSL | 146 |
| K7-Polybia-CP-NH2 | ILGTILKLNKSL | 147 |
| K7-Polybia-CP-NH2 | ILGTILKLPKSL | 148 |
| K7-Polybia-CP-NH2 | ILGTILKLQKSL | 149 |
| K7-Polybia-CP-NH2 | ILGTILKLRKSL | 150 |
| K7-Polybia-CP-NH2 | ILGTILKLSKSL | 151 |
| K7-Polybia-CP-NH2 | ILGTILKLTKSL | 152 |
| K7-Polybia-CP-NH2 | ILGTILKLVKSL | 153 |
| K7-Polybia-CP-NH2 | ILGTILKLWKSL | 154 |
| K7-Polybia-CP-NH2 | ILGTILKLYKSL | 155 |
| K7-Polybia-CP-NH2 | ILGTILKLLASL | 156 |
| K7-Polybia-CP-NH2 | ILGTILKLLCSL | 157 |
| K7-Polybia-CP-NH2 | ILGTILKLLDSL | 158 |
| K7-Polybia-CP-NH2 | ILGTILKLLESL | 159 |
| K7-Polybia-CP-NH2 | ILGTILKLLFSL | 160 |
| K7-Polybia-CP-NH2 | ILGTILKLLGSL | 161 |
| K7-Polybia-CP-NH2 | ILGTILKLLHSL | 162 |
| K7-Polybia-CP-NH2 | ILGTILKLLISL | 163 |
| K7-Polybia-CP-NH2 | ILGTILKLLLSL | 164 |
| K7-Polybia-CP-NH2 | ILGTILKLLMSL | 165 |
| K7-Polybia-CP-NH2 | ILGTILKLLNSL | 166 |
| K7-Polybia-CP-NH2 | ILGTILKLLPSL | 167 |
| K7-Polybia-CP-NH2 | ILGTILKLLQSL | 168 |
| K7-Polybia-CP-NH2 | ILGTILKLLRSL | 169 |
| K7-Polybia-CP-NH2 | ILGTILKLLSSL | 170 |
| K7-Polybia-CP-NH2 | ILGTILKLLTSL | 171 |
| K7-Polybia-CP-NH2 | ILGTILKLLVSL | 172 |
| K7-Polybia-CP-NH2 | ILGTILKLLWSL | 173 |
| K7-Polybia-CP-NH2 | ILGTILKLLYSL | 174 |
| K7-Polybia-CP-NH2 | ILGTILKLLKAL | 175 |
| K7-Polybia-CP-NH2 | ILGTILKLLKCL | 176 |
| K7-Polybia-CP-NH2 | ILGTILKLLKDL | 177 |
| K7-Polybia-CP-NH2 | ILGTILKLLKEL | 178 |
| K7-Polybia-CP-NH2 | ILGTILKLLKFL | 179 |
| K7-Polybia-CP-NH2 | ILGTILKLLKGL | 180 |
| K7-Polybia-CP-NH2 | ILGTILKLLKHL | 181 |
| K7-Polybia-CP-NH2 | ILGTILKLLKIL | 182 |
| K7-Polybia-CP-NH2 | ILGTILKLLKKL | 183 |
| K7-Polybia-CP-NH2 | ILGTILKLLKLL | 184 |
| K7-Polybia-CP-NH2 | ILGTILKLLKML | 185 |
| K7-Polybia-CP-NH2 | ILGTILKLLKNL | 186 |
| K7-Polybia-CP-NH2 | iLGTILKLLKPL | 187 |
| K7-Polybia-CP-NH2 | ILGTILKLLKQL | 188 |
| K7-Polybia-CP-NH2 | ILGTILKLLKRL | 189 |
| K7-Polybia-CP-NH2 | ILGTILKLLKTL | 190 |
| K7-Polybia-CP-NH2 | ILGTILKLLKVL | 191 |
| K7-Polybia-CP-NH2 | ILGTILKLLKWL | 192 |
| K7-Polybia-CP-NH2 | ILGTILKLLKYL | 193 |
| K7-Polybia-CP-NH2 | ILGTILKLLKSA | 194 |
| K7-Polybia-CP-NH2 | ILGTILKLLKSC | 195 |
| K7-Polybia-CP-NH2 | ILGTILKLLKSD | 196 |
| K7-Polybia-CP-NH2 | ILGTILKLLKSE | 197 |
| K7-Polybia-CP-NH2 | ILGTILKLLKSF | 198 |
| K7-Polybia-CP-NH2 | ILGTILKLLKSG | 199 |
| K7-Polybia-CP-NH2 | ILGTILKLLKSH | 200 |
| K7-Polybia-CP-NH2 | ILGTILKLLKSI | 201 |
| K7-Polybia-CP-NH2 | ILGTILKLLKSK | 202 |
| K7-Polybia-CP-NH2 | ILGTILKLLKSM | 203 |
| K7-Polybia-CP-NH2 | ILGTILKLLKSN | 204 |
| K7-Polybia-CP-NH2 | ILGTILKLLKSP | 205 |
| K7-Polybia-CP-NH2 | ILGTILKLLKSQ | 206 |
| K7-Polybia-CP-NH2 | ILGTILKLLKSE | 207 |
| K7-Polybia-CP-NH2 | ILGTILKLLKSS | 208 |
| K7-Polybia-CP-NH2 | ILGTILKLLKST | 209 |
| K7-Polybia-CP-NH2 | ILGTILKLLKSV | 210 |
| K7-Polybia-CP-NH2 | ILGTILKLLKSW | 211 |
| K7-Polybia-CP-NH2 | ILGTILKLLKSY | 212 |
| L10-Decoralin-NH2 | ALLSLIRKLLT | 213 |
| L10-Decoralin-NH2 | CLLSLIRKLLT | 214 |
| L10-Decoralin-NH2 | DLLSLIRKLLT | 215 |
| L10-Decoralin-NH2 | ELLSLIRKLLT | 216 |
| L10-Decoralin-NH2 | FLLSLIRKLLT | 217 |
| L10-Decoralin-NH2 | GLLSLIRKLLT | 218 |
| L10-Decoralin-NH2 | HLLSLIRKLLT | 219 |
| L10-Decoralin-NH2 | ILLSLIRKLLT | 220 |
| L10-Decoralin-NH2 | KLLSLIRKLLT | 221 |
| L10-Decoralin-NH2 | LLLSLIRKLLT | 222 |
| L10-Decoralin-NH2 | MLLSLIRKLLT | 223 |
| L10-Decoralin-NH2 | NLLSLIRKLLT | 224 |
| L10-Decoralin-NH2 | PLLSLIRKLLT | 225 |
| L11-Decoralin-NH2 | QLLSLTRKLLT | 226 |
| L10-Decoralin-NH2 | RLLSLIRKLLT | 227 |
| L10-Decoralin-NH2 | TLLSLIRKLLT | 228 |
| L10-Decoralin-NH2 | VLLSLIRKLLT | 229 |
| L10-Decoralin-NH2 | WLLSLIRKLLT | 230 |
| L10-Decoralin-NH2 | YLLSLIRKLLT | 231 |
| L10-Decoralin-NH2 | SALSLIRKLLT | 232 |
| L10-Decoralin-NH2 | SCLSLIRKLLT | 233 |
| L11-Decoralin-NH2 | SDLSLIRKLLT | 234 |
| L10-Decoralin-NH2 | SELSLIRKLLT | 235 |
| L10-Decoralin-NH2 | SFLSLIRKLLT | 236 |
| L10-Decoralin-NH2 | SGLSLIRKLLT | 237 |
| L10-Decoralin-NH2 | SHLSLIRKLLT | 238 |
| L10-Decoralin-NH2 | SILSLIRKLLT | 239 |
| L10-Decoralin-NH2 | SKLSLIRKLLT | 240 |
| L10-Decoralin-NH2 | SMLSLIRKLLT | 241 |
| L10-Decoralin-NH2 | SNLSLIRKLLT | 242 |
| L10-Decoralin-NH2 | SELSLIRKLLT | 243 |
| L10-Decoralin-NH2 | SQLSLIRKLLT | 244 |
| L10-Decoralin-NH2 | SRLSLIRKLLT | 245 |
| L10-Decoralin-NH2 | SSLSLIRKLLT | 246 |
| L10-Decoralin-NH2 | STLSLIRKLLT | 247 |
| L10-Decoralin-NH2 | SVLSLIRKLLT | 248 |
| L10-Decoralin-NH2 | SWLSLIRKLLT | 249 |
| L10-Decoralin-NH2 | SYLSLIRKLLT | 250 |
| L10-Decoralin-NH2 | SLASLIRKLLT | 251 |
| L10-Decoralin-NH2 | SLCSLIRKLLT | 252 |
| L10-Decoralin-NH2 | SLDSLIRKLLT | 253 |
| L10-Decoralin-NH2 | SLESLIRKLLT | 254 |
| L10-Decoralin-NH2 | SLFSLIRKLLT | 255 |
| L10-Decoralin-NH2 | SLGSLIRKLLT | 256 |
| L10-Decoralin-NH2 | SLHSLIRKLLT | 257 |
| L10-Decoralin-NH2 | SLISLIRKLLT | 258 |
| L10-Decoralin-NH2 | SLKSLIRKLLT | 259 |
| L10-Decoralin-NH2 | SLMSLIRKLLT | 260 |
| L10-Decoralin-NH2 | SLNSLIRKLLT | 261 |
| L10-Decoralin-NH2 | SLPSLIRKLLT | 262 |
| L10-Decoralin-NH2 | SLQSLIRKLLT | 263 |
| L11-Decoralin-NH2 | SLRSLIRKLLT | 264 |
| L10-Decoralin-NH2 | SLSSLIRKLLT | 265 |
| L10-Decoralin-NH2 | SLTSLIRKLLT | 266 |
| L10-Decoralin-NH2 | SLVSLIRKLLT | 267 |
| L10-Decoralin-NH2 | SLWSLIRKLLT | 268 |
| L10-Decoralin-NH2 | SLYSLIRKLLT | 269 |
| L10-Decoralin-NH2 | SLLALIRKLLT | 270 |
| L10-Decoralin-NH2 | SLLCLIRKLLT | 271 |
| L11-Decoralin-NH2 | SLLDLIRKLLT | 272 |
| L10-Decoralin-NH2 | SLLELIRKLLT | 273 |
| L10-Decoralin-NH2 | SLLFLIRKLLT | 274 |
| L10-Decoralin-NH2 | SLLGLIRKLLT | 275 |
| L10-Decoralin-NH2 | SLLMLIRKLLT | 276 |
| L10-Decoralin-NH2 | SLLILIRKLLT | 277 |
| L10-Decoralin-NH2 | SLLKLIRKLLT | 278 |
| L10-Decoralin-NH2 | SLLLLIRKLLT | 279 |
| L10-Decoralin-NH2 | SLLMLIRKLLT | 280 |
| L10-Decoralin-NH2 | SLLNLIRKLLT | 281 |
| L10-Decoralin-NH2 | SLLPLIRKLLT | 282 |
| L10-Decoralin-NH2 | SLLQLIRKLLT | 283 |
| L10-Decoralin-NH2 | SLLRLIRKLLT | 284 |
| L10-Decoralin-NH2 | SLLTLIRKLLT | 285 |
| L10-Decoralin-NH2 | SLLVLIRKLLT | 286 |
| L10-Decoralin-NH2 | SLLWLIRKLLT | 287 |
| L10-Decoralin-NH2 | SLLVLIRKLLT | 288 |
| L10-Decoralin-NH2 | SLLSATRKLLT | 289 |
| L10-Decoralin-NH2 | SLLSCIRKLLT | 290 |
| L10-Decoralin-NH2 | SLLSDIRKLLT | 291 |
| L10-Decoralin-NH2 | SLLSEIRKLLT | 292 |
| L10-Decoralin-NH2 | SLLSFTRKLLT | 293 |
| L10-Decoralin-NH2 | SLLSGIRKLLT | 294 |
| L10-Decoralin-NH2 | SLLSHIRKLLT | 295 |
| L10-Decoralin-NH2 | SLLSLIRKLLT | 296 |
| L10-Decoralin-NH2 | SLLSKIRKLLT | 297 |
| L10-Decoralin-NH2 | SLLSMIRKLLT | 298 |
| L10-Decoralin-NH2 | SLLSNIRKLLT | 299 |
| L10-Decoralin-NH2 | SLLSPIRKLLT | 300 |
| L10-Decoralin-NH2 | SLLSQIRKLLT | 301 |
| L11-Decoralin-NH2 | SLLSR1RKLLT | 302 |
| L10-Decoralin-NH2 | SLLSSIRKLLT | 303 |
| L10-Decoralin-NH2 | SLLSTIRKLLT | 304 |
| L10-Decoralin-NH2 | SLLSVIRKLLT | 305 |
| L10-Decoralin-NH2 | SLLSWIRKLLT | 306 |
| L10-Decoralin-NH2 | SLLSYIRKLLT | 307 |
| L10-Decoralin-NH2 | SLLSLARKLLT | 308 |
| L10-Decoralin-NH2 | SLLSLCRKLLT | 309 |
| L11-Decoralin-NH2 | SLLSLDRKLLT | 310 |
| L10-Decoralin-NH2 | SLLSLERKLLT | 311 |
| L10-Decoralin-NH2 | SLLSLFRKLLT | 312 |
| L10-Decoralin-NH2 | SLLSLGRKLLT | 313 |
| L10-Decoralin-NH2 | SLLSLHRKLLT | 314 |
| L10-Decoralin-NH2 | SLLSLKRKLLT | 315 |
| L10-Decoralin-NH2 | SLLSLLRKLLT | 316 |
| L10-Decoralin-NH2 | SLLSLMRKLLT | 317 |
| L10-Decoralin-NH2 | SLLSLNRKLLT | 318 |
| L10-Decoralin-NH2 | SLLSLFRKLLT | 319 |
| L10-Decoralin-NH2 | SLLSLQRKLLT | 320 |
| L10-Decoralin-NH2 | SLLSLRRKLLT | 321 |
| L10-Decoralin-NH2 | SLLSLSRKLLT | 322 |
| L10-Decoralin-NH2 | SLLSLTRKLLT | 323 |
| L10-Decoralin-NH2 | SLLSLVRKLLT | 324 |
| L10-Decoralin-NH2 | SLLSLWRKLLT | 325 |
| L10-Decoralin-NH2 | SLLSLYRKLLT | 326 |
| L10-Decoralin-NH2 | SLLSLIAKLLT | 327 |
| L10-Decoralin-NH2 | SLLSLICKLLT | 328 |
| L10-Decoralin-NH2 | SLLSLIDKLLT | 329 |
| L10-Decoralin-NH2 | SLLSLIEKLLT | 330 |
| L10-Decoralin-NH2 | SLLSLIFKLLT | 331 |
| L10-Decoralin-NH2 | SLLSLIGKLLT | 332 |
| L10-Decoralin-NH2 | SLLSLIHKLLT | 333 |
| L10-Decoralin-NH2 | SLLSLIIKLLT | 334 |
| L10-Decoralin-NH2 | SLLSLIKKLLT | 335 |
| L10-Decoralin-NH2 | SLLSLILKLLT | 336 |
| L10-Decoralin-NH2 | SLLSLIMKLLT | 337 |
| L10-Decoralin-NH2 | SLLSLINKLLT | 338 |
| L10-Decoralin-NH2 | SLLSLIPKLLT | 339 |
| L11-Decoralin-NH2 | SLLSLIQKLLT | 340 |
| L10-Decoralin-NH2 | SLLSLISKLLT | 341 |
| L10-Decoralin-NH2 | SLLSLITKLLT | 342 |
| L10-Decoralin-NH2 | SLLSLIVKLLT | 343 |
| L10-Decoralin-NH2 | SLLSLIWKLLT | 344 |
| L10-Decoralin-NH2 | SLLSLIYKLLT | 345 |
| L10-Decoralin-NH2 | SLLSLIRKALT | 346 |
| L10-Decoralin-NH2 | SLLSLIRKCLT | 347 |
| L11-Decoralin-NH2 | SLLSLIRKDLT | 348 |
| L10-Decoralin-NH2 | SLLSLIRKELT | 349 |
| L10-Decoralin-NH2 | SLLSLIRKFLT | 350 |
| L10-Decoralin-NH2 | SLLSLIRKGLT | 351 |
| L10-Decoralin-NH2 | SLLSLIRKHLT | 352 |
| L10-Decoralin-NH2 | SLLSLIRKILT | 353 |
| L10-Decoralin-NH2 | SLLSLIRKKLT | 354 |
| L10-Decoralin-NH2 | SLLSLIRKMLT | 355 |
| L10-Decoralin-NH2 | SLLSLIRKNLT | 356 |
| L10-Decoralin-NH2 | SLLSLIRKPLT | 357 |
| L10-Decoralin-NH2 | SLLSLIRKQLT | 358 |
| L10-Decoralin-NH2 | SLLSLIRKRLT | 359 |
| L10-Decoralin-NH2 | SLLSLIRKSLT | 360 |
| L10-Decoralin-NH2 | SLLSLIRKTLT | 361 |
| L10-Decoralin-NH2 | SLLSLIRKVLT | 362 |
| L10-Decoralin-NH2 | SLLSLIRKWLT | 363 |
| L10-Decoralin-NH2 | SLLSLIRKYLT | 364 |
| L10-Decoralin-NH2 | SLLSLIRKLLA | 365 |
| L10-Decoralin-NH2 | SLLSLIRKLLC | 366 |
| L10-Decoralin-NH2 | SLLSLIRKLLD | 367 |
| L10-Decoralin-NH2 | SLLSLIRKLLE | 368 |
| L10-Decoralin-NH2 | SLLSLIRKLLF | 369 |
| L10-Decoralin-NH2 | SLLSLIRKLLG | 370 |
| L10-Decoralin-NH2 | SLLSLIRKLLH | 371 |
| L10-Decoralin-NH2 | SLLSLIRKLLI | 372 |
| L10-Decoralin-NH2 | SLLSLIRKLLK | 373 |
| L10-Decoralin-NH2 | SLLSLIRKLLL | 374 |
| L10-Decoralin-NH2 | SLLSLIRKLLM | 375 |
| L10-Decoralin-NH2 | SLLSLIRKLLN | 376 |
| L10-Decoralin-NH2 | SLLSLIRKLLP | 377 |
| L10-Decoralin-NH2 | SLLSLIRKLLQ | 378 |
| L10-Decoralin-NH2 | SLLSLIRKLLR | 379 |
| L10-Decoralin-NH2 | SLLSLIRKLLS | 380 |
| L10-Decoralin-NH2 | SLLSLIRKLLV | 381 |
| L10-Decoralin-NH2 | SLLSLIRKLLW | 382 |
| L10-Decoralin-NH2 | SLLSLIRKLLY | 383 |

### Discussion.

AMPs represent promising alternatives to conventional antibiotics to combat the global health problem of antibiotic resistance (Mahlapuu M., et al., Front. Cell. Infect. Microbiol. 6, 1-12 (2016); de la Fuente-Nunez C. et al., Curr. Opin. Microbiol. doi:10.1016/j.mib.2017.05.014 (2017)). However, their development is limited by the lack of methods for cost-effective and rational design (Mulder K. C. L. et al., Curr. Protein Pept. Sci. 14, 556-567 (2013); Bradshaw J. P. BioDrugs. 17, 233-240 (2003); da Costa J. P. et al., Appl. Microbiol. Biotechnol. 99, 2023-2040 (2015); Fjell C. D. et al., Nat. Rev. Drug Discov. 11, (2011)). Although some alternative methods to overcome these limitations have been proposed (Li Y. Protein Expr. Purif. 80, 260-267 (2011); Ong Z. Y. et al., Adv. Drug Deliv. Rev. 78, 28-45 (2014); Zhao C. X. et al., Biotechnol. Bioeng. 112, 957-964 (2015)), the SAR of these agents is far from understood, which would provide a more substantial basis for their rational design and accelerate their translation to the clinic.

Here, a systematic SAR design approach is described aimed at revealing the sequence requirements for antimicrobial activity of a natural wasp venom AMP (Souza B. M. et al., Peptides 26, 2157-2164 (2005)) and several of its derivatives. Through single-residue substitutions guided by identified physicochemical activity determinants, peptide antibiotics were generated with anti-infective potential in a mouse model.

Pol-CP—NH₂ is a chemotactic peptide from the venom of a tropical species of wasp that presents 12 residues typical of peptides found in these wasp species (Souza B. M. et al., Peptides 26, 2157-2164 (2005)). Wasp venom peptides usually present characteristic motifs, such as a Phe-Leu-Pro tripeptide at the amino terminal side, which are thought to be responsible for their mechanism of action. Pol-CP—NH₂, however, lacks these specific sequence patterns, which may explain its decreased antimicrobial activity compared to other wasp venom peptides such as mastoparan and VesCP (Souza B. M. et al., Peptides 26, 2157-2164 (2005); Nagashima K. et al., Biochem. Biophys. Res. Commun. 168, 844-849 (1990)). Also unlike other wasp venom peptides, Pol-CP—NH₂ lacks a central cationic Lys residue in its seventh position (Nagashima K. et al., Biochem. Biophys. Res. Commun. 168, 844-849 (1990)). Pol-CP—NH₂ does contain a Lys residue in its tenth position, like its analog protonectin. The main structural difference between protonectin and Pol-CP—NH₂ is the replacement of the eleventh residue in protonectin (Gly) by a Ser in the Pol-CP—NH₂ sequence. The differences between Pol-CP—NH₂ and other mastoparan-like peptides does not prevent it from presenting chemotactic activity. Pol-CP—NH₂ was described as cause of mast cell degranulation activity reduction, mast cell lysis, besides of inducing chemotaxis of polymorphonucleated leukocytes, characteristics usually observed for wasp venom mastoparan-like peptides (Nagashima K. et al., Biochem. Biophys. Res. Commun. 168, 844-849 (1990)).

MIC (FIGS. 2A-2E), CFU counts (FIGS. 10A-10B), and CD spectra (FIGS. 3A-3C) assays using Ala-scan analogs revealed that positions 3 (Gly), 4 (Thr), 6 (Leu), 7 (Gly), and 11 (Ser) were residues with side chains that did not substantially contribute to structure and function, whereas positions 5 (Ile) and 10 (Lys) were identified as key determinants of structure and antimicrobial function. Thus, the hydrophilic residues present in Pol-CP—NH₂ (FIG. 2E) were not important for the peptide to adopt a helical structure or for antimicrobial function, with the exception of the only charged residue (Lys). On the other hand, the hydrophobic residues present in the wild-type peptide appear to be vital for peptide structure because of their aliphatic side chains and the hydrophobic interactions of these side chains, which enable the unstructured-to-helix transition in an environment, such as the bacterial membrane or TFE/water, that favors structuring of the peptide (FIGS. 3A-3C).

To test the importance of the hydrophilic residues and increased charge in structure-function, synthetic analogs were engineered. Two of these ([Lys]⁴-Pol-CP—NH₂ and [Lys]'-Pol-CP—NH₂), which had insertions in the hydrophilic face at positions that would keep the hydrophobicity and hydrophobic moment within the optimal range (FIGS. 2C-2D), impacted favorably both structure and antimicrobial activity (FIG. 2E and FIG. 3C). One of the analogs ([Lys]⁵-Pol-CP—NH₂) showed decreased antimicrobial activity because a positive charged residue was inserted in the hydrophobic face leading to decreased hydrophobicity and hydrophobic moment. Results obtained with these analogs show that, even with the insertion of a charged residue, the position of the insertion and the overall structure are more important to antimicrobial activity than increased net positive charge, as described for other cationic amphipathic AMPs (Taniguchi M. et al., Biopolymers 102, 58-68 (2014); Lee J. K. et al., Biochim. Biophys. Acta. Biomembr. 1828, 443-454 (2013); Du Q. et al., Int. J. Biol. Sci. 10, 1097-1107 (2014)).

The impact of the introduction of charge via the insertion of Lys residues in positions 4, 5, and 7 was also predicted in the initial experiments (FIGS. 4A-4C and FIGS. 5A-5D). Increasing helical content led to increased antimicrobial activity against a larger set of Gram-positive and Gram-negative bacteria and fungi. When the insertion was made within the hydrophilic face, enhanced antimicrobial activity was observed; the opposite effect was obtained when the substitution was made within the hydrophobic face of the peptide.

To analyze the combined effect of charge and the importance of the residues' side chains on the hydrophobic face, other analogs with double ([Leu]⁵-[Lys]⁹-Pol-CP—NH₂) and triple substitutions ([Glu]³-[Lys]⁵-[Glu]¹²-Pol-CP—NH₂) were synthesized based on two-dimensional helical wheels (FIG. 12). These modifications were predicted to change the physicochemical features as much as single mutations at those positions with slight changes in their side chain size, and a single substitution with an aromatic hydrophobic residue to increase hydrophobicity in the middle of the hydrophobic face of the amphipathic structure ([Phe]⁹-Pol-CP—NH₂). The substitution was made in position 9 as this is the closest position to the center of the hydrophobic face that did not alter the structure when Leu was replaced by Ala (FIGS. 2A-2E and FIG. 9). The insertion of a Phe residue in position 9 led to increased predicted hydrophobic moment. This insertion served to check for cytotoxicity effects, as aromatic residues are known for their cytotoxic propensity due to enhanced hydrophobic interactions with lipids (Lee J. K. et al., Biochim. Biophys. Acta—Biomembr. 1828, 443-454 (2013)). In addition, a Gly-substituted analog ([Gly]¹-Pol-CP—NH₂) was designed, as Gly is commonly the first residue in AMPs (Zelezetsky I. and Tossi A. Biochim. Biophys. Acta—Biomembr. 1758, 1436-1449 (2006)), and deleted the last residue (Des[Leu]¹²-Pol-CP—NH₂), which changed peptide size and hydrophilic/hydrophobic ratio.

Results obtained with the newly designed analogs (FIGS. 4A-4C) confirmed the hydrophobicity and hydrophobic moment optimal ranges observed previously (FIGS. 2A-2E), although some exceptions were identified (FIGS. 5C-5D). Increasing the helical content consistently led to improved antimicrobial activity (FIG. 5B) in line with the previous data (FIG. 3B). Collectively, tuning the helical content and net positive charge in specific positions (hydrophilic face) within the wild-type peptide enhanced its antimicrobial activity more predictably than modulating hydrophobicity.

A critical design property of AMPs is ensuring their specificity towards microorganisms, while minimizing unwanted toxicity against human cells. To check the toxicity of the second generation of Pol-CP—NH₂ derivatives, assays were performed using red blood cells either untreated or exposed to peptides (0-100 [symbol] mol L⁻¹—FIG. 6A). Besides the wild type, only the most active ([Lys]⁷-Pol-CP—NH₂) and the most hydrophobic ([Phe]⁹-Pol-CP—NH₂) analogs were hemolytic. The most active derivative, [Lys]⁷-Pol-CP—NH₂, was hemolytic at 12.5 [symbol] mol L⁻¹, a concentration substantially higher than its MIC against all the microorganisms tested (FIGS. 5A-5D and FIGS. 6A-6B). However, [Phe]⁹-Pol-CP—NH₂ was as hemolytic as the wild-type (FIG. 6A) at doses corresponding to its average MIC (˜50 [symbol] mol L⁻¹) (FIG. 5A). The selectivity index (SI) of the hemolytic peptides was calculated as the ratio between the concentrations leading to 50% lysis of human erythrocytes and the average of the minimum concentration inhibiting bacterial growth of twelve different strains (SI═HC₅₀/MIC)⁶², indicating how selective were the peptides. The most active analog, [Lys]⁷-Pol-CP—NH₂, presented a SI of 9.2, which was greater than the one presented by the analog [Phe]⁹-Pol-CP—NH₂ (2.5) and the wild-type (3.1). Indicating that even hemolytic in lower concentrations, [Lys]⁷-Pol-CP—NH₂ was the most selective peptide towards a large variety of microorganisms including Gram-positive, Gram-negative and fungi, due to its higher antimicrobial activity. To further assess the toxicity profile of the peptides, lead compounds were subjected to cytotoxicity assays using HEK293 cells (human embryonic kidney cells). The cells were exposed to increasing doses of peptides (0-64 [symbol] mol L⁻¹—FIGS. 7A-7B), and cytotoxicity correlated with increased helical content.

The presence of charged residues on cationic amphipathic AMPs usually correlates with susceptibility to degradation by proteases. Being unstructured in water or saline media, these AMPs are easily cleaved by peptidases. The stability of Pol-CP—NH₂ and analogs in fetal bovine serum wash checked for six hours and a small difference was observed in their resistance to degradation (FIG. 6B). The most resistant peptides were those with higher helical content.

Among the microorganisms studied, P. aeruginosa is a pathogenic Gram-negative bacterium responsible for pneumonia (El Solh A. A. et al., Am. J. Respir. Crit. Care Med. 178, 513-519 (2008)) and for infections of the urinary tract (Newman J. W. et al., FEMS Microbiol. Lett. 364, fnx124-fnx124 (2017)), gastrointestinal tissue (Yeung C. K. and Lee K. H. J. Paediatr. Child Health 34, 584-587 (1998)), skin and soft tissues (Nagoba B. et al., Wound Med. 19, 5-9 (2017); Dryden M. S. J. Antimicrob. Chemother. 65, iii35-iii44 (2010); Buivydas A. et al., FEMS Microbiol. Lett. 343, 183-189 (2013)) and is very common in patients with cystic fibrosis (Stefani S. et al., Int. J. Med. Microbiol. 307, 353-362 (2017)). Like other bacteria, P. aeruginosa is becoming resistant to common antibiotics (Stefani S. et al., Int. J. Med. Microbiol. 307, 353-362 (2017)), and AMPs have been proposed as an alternative treatment to combat such infections (Chen C. et al., Sci. Rep. 7, 8548 (2017)).

The skin infection mouse model used here involved inducing a P. aeruginosa abscess and treating mice with a single dose of the selected peptides at low concentrations (4 [symbol] mol L⁻¹) that did not induce hemolysis (FIGS. 6A-6B) or cytotoxicity (FIGS. 7A-7B). The effect of peptides on bacterial load in the infection site was assessed (FIGS. 8A-8B). The analogs used in these assays were some of the lead peptides, e.g., peptides with high activity against P. aeruginosa (FIGS. 2A-2E and FIGS. 13A-13B—[Ala]⁷-Pol-CP—NH₂, [Ala]¹¹-Pol-CP—NH₂, [Lys]⁴-Pol-CP—NH₂, [Lys]⁷-Pol-CP—NH₂ and [Gly]¹-Pol-CP—NH₂,) and some less active analogs (FIG. 2B—[Ala]³-Pol-CP—NH₂ and [Ala]⁵-Pol-CP—NH₂), in addition to the wild type. The antimicrobial activity observed in vivo (FIG. 8B) correlated with that obtained in vitro (FIGS. 2A-2E and FIGS. 5A-5D). The most active AMPs from the second-generation library had +3 net positive charge and exhibited superior activity compared to the wild type and the Ala-scan active analogs. As expected, the peptides used as negative controls ([Ala]³-Pol-CP—NH₂ and [Ala]⁵-Pol-CP—NH₂) (FIGS. 2A-2E) did not kill bacteria in vivo (FIG. 8B). [Ala]¹¹-Pol-CP—NH₂ was not active at the concentration tested (4 [symbol] mol L⁻¹), which is not entirely surprising as its MIC value against P. aeruginosa is 4-fold higher (16 [symbol] mol L⁻¹—FIG. 2B).

To show the suitability of the lead peptide [Lys]⁷-Pol-CP—NH₂ as a novel peptide antibiotic, its anti-infective activity was tested against P. aeruginosa using the mouse model (FIG. 8A). Because the WT and [Lys]⁷-Pol-CP—NH₂ were toxic at 64 [symbol] mol L⁻¹, experiments were conducted thoroughly and any signs of toxicity in vivo were observed, what was confirmed by body weight measurements of the mice (FIG. 8C). Peptide treatment nearly sterilized the infection (FIG. 8D), thereby demonstrating the potential of this synthetic peptide as a novel antimicrobial.

Disclosed herein is a physicochemical feature-guided design of antimicrobial peptides that is a useful tool for identifying functional determinants and designing novel synthetic peptide antibiotics. Using such an approach (Ala-scan and residue probability in determined positions), a naturally occurring AMP has been converted from an AMP with lower activity against Gram-negative bacteria (Souza B. M. et al., Peptides 26, 2157-2164 (2005)), into potent variants capable of killing bacteria at nanomolar doses and displaying anti-infective activity in an animal models. This study is an example of how to design small cationic amphitathic peptides to optimize biological activities and selectivity. The principles and approaches exploited here can be applied to other structure-activity studies in order to rationalize and better understand the role of physicochemical features and which approaches fit better to each family of peptides.

### REFERENCES

1. CDC. Antibiotic resistance threats in the United States, 2013. Current 114 (2013). doi:CS239559-B.
2. Walsh, C. Molecular mechanisms that confer antibacterial drug resistance. Nature. 406, 775-781 (2000).
3. Arora, G., Sajid, A. & Chandra, V. Drug Resistance in Bacteria, Fungi, Malaria, and Cancer. Springer. doi:10.1007/978-3-319-48683-3 (2017).
4. Mahlapuu, M., Hakansson, J., Ringstad, L. & Björn, C. Antimicrobial Peptides: An Emerging Category of Therapeutic Agents. Front. Cell. Infect. Microbiol. 6, 1-12 (2016).
5. Pendleton, J. N., Gorman, S. P. & Gilmore, B. F. Clinical relevance of the ESKAPE pathogens. Expert Rev. Anti. Infect. Ther. 11,297-308 (2013).
6. de la Fuente-Nunez, C., Tones, M. D., Mojica, F. J. & Lu, T. K. Next-generation precision antimicrobials: towards personalized treatment of infectious diseases. Curr. Opin. Microbiol. (2017). doi:10.1016/j.mib.2017.05.014.
7. Melo, M. N., Ferre, R. & Castanho, M. A. R. B. Antimicrobial peptides: linking partition, activity and high membrane-bound concentrations. Nat. Rev. Microbiol. 7, 245-250 (2009).
8. Andersson, D. I., Hughes, D. & Kubicek-Sutherland, J. Z. Mechanisms and consequences of bacterial resistance to antimicrobial peptides. Drug Resist. Updat. 26, 43-57 (2016).
9. Perumal Samy, R., Stiles, B. G., Franco, O. L., Sethi, G. & Lim, L. H. K. Animal venoms as antimicrobial agents. Biochemical Pharmacology 134, (2017).
10. Hancock, R. E. W. Cationic antimicrobial peptides: towards clinical applications. Expert Opin. Investig. Drugs 9,1723-1729 (2000).
11. Lifson, S. & Roig, A. On the Theory of Helix-Coil Transition in Polypeptides. J. Chem. Phys. 34,1963-1974 (1961).
12. Zimm, B. H. & Bragg, J. K. Theory of the Phase Transition between Helix and Random Coil in Polypeptide Chains. J. Chem. Phys. 31,526-535 (1959).
13. Nguyen, L. T., Haney, E. F. & Vogel, H. J. The expanding scope of antimicrobial peptide structures and their modes of action. Trends Biotechnol. 29,464-472 (2011).
14. Brogden, K. A. Antimicrobial peptides: pore formers or metabolic inhibitors in bacteria? Nat. Rev. Microbiol. 3,238 (2005).
15. Lohner, K. New strategies for novel antibiotics: peptides targeting bacterial cell membranes. Gen. Physiol. Biophys. 28,105-116 (2009).
16. Epand, R. M. & Epand, R. F. Bacterial membrane lipids in the action of antimicrobial agents. J. Pept. Sci. 17,298-305 (2011).
17. Rokitskaya, T. I., Kolodkin, N. I., Kotova, E. A. & Antonenko, Y. N. Indolicidin action on membrane permeability: Carrier mechanism versus pore formation. Biochim. Biophys. Acta—Biomembr. 1808,91-97 (2011).
18. Chan, D. I., Prenner, E. J. & Vogel, H. J. Tryptophan- and arginine-rich antimicrobial peptides: Structures and mechanisms of action. Biochim. Biophys. Acta—Biomembr. 1758, 1184-1202 (2006).
19. Gifford, J. L., Hunter, H. N. & Vogel, H. J. Lactoferricin: a lactoferrin-derived peptide with antimicrobial, antiviral, antitumor and immunological properties. Cell. Mol. Life Sci. 62, 2588-98 (2005).
20. Haney, E. F., Nathoo, S., Vogel, H. J. & Prenner, E. J. Induction of non-lamellar lipid phases by antimicrobial peptides: a potential link to mode of action. Chem. Phys. Lipids 163, 82-93 (2010).
21. Le, C.-F., Gudimella, R., Razali, R., Manikam, R. & Sekaran, S. D. Transcriptome analysis of Streptococcus pneumoniae treated with the designed antimicrobial peptides, DM3. Sci. Rep. 6, 26828 (2016).
22. Huang, N. et al. Sirtuin 6 plays an oncogenic role and induces cell autophagy in esophageal cancer cells. Tumor Biol. 39, 1010428317708532 (2017).
23. Park, C. B., Kim, H. S. & Kim, S. C. Mechanism of Action of the Antimicrobial Peptide Buforin II: Buforin II Kills Microorganisms by Penetrating the Cell Membrane and Inhibiting Cellular Functions. Biochem. Biophys. Res. Commun. 244, 253-257 (1998).
24. Krizsan, A., Prahl, C., Goldbach, T., Knappe, D. & Hoffmann, R. Short Proline-Rich Antimicrobial Peptides Inhibit Either the Bacterial 70S Ribosome or the Assembly of its Large 505 Subunit. Chembiochem A Eur. J. Chem. Biol. 16, 2304-2308 (2015).
25. de Kruijff, B., van Dam, V. & Breukink, E. Lipid II: A central component in bacterial cell wall synthesis and a target for antibiotics. Prostaglandins, Leukot. Essent. Fat. Acids 79, 117-121 (2008).
26. Subbalakshmi, C. & Sitaram, N. Mechanism of antimicrobial action of indolicidin. FEMS Microbiol. Lett. 160, 91-96 (1998).
27. Haney, E. F. et al. Mechanism of action of puroindoline derived tryptophan-rich antimicrobial peptides. Biochim. Biophys. Acta—Biomembr. 1828, 1802-1813 (2013).
28. da Costa, J. P., Cova, M., Ferreira, R. & Vitorino, R. Antimicrobial peptides: an alternative for innovative medicines? Appl. Microbiol. Biotechnol. 99, 2023-2040 (2015).
29. Lee, S. H., Baek, J. H. & Yoon, K. A. Differential properties of venom peptides and proteins in solitary vs. Social hunting wasps. Toxins (Basel). 8, 1-29 (2016).
30. Souza, B. M. et al. Structural and functional characterization of two novel peptide toxins isolated from the venom of the social wasp Polybia paulista. Peptides 26, 2157-2164 (2005).
31. Gautier, R., Douguet, D., Antonny, B. & Drin, G. HELIQUEST: A web server to screen sequences with specific a-helical properties. Bioinformatics 24, 2101-2102 (2008).
32. Greenfield, N. J. Applications of circular dichroism in protein and peptide analysis. Trends Anal. Chem. 18, 236-244 (1999).
33. Pedron, C. N. et al. Novel designed VmCT1 analogs with increased antimicrobial activity. Eur. J. Med. Chem. 126, 456-463 (2017).
34. Torres, M. D. T. et al. Decoralin Analogs with Increased Resistance to Degradation and Lower Hemolytic Activity. ChemistrySelect 2, 18-23 (2017).
35. Torres, M. D. T. et al. Antimicrobial activity of leucine-substituted decoralin analogs with lower hemolytic activity. J. Pept. Sci. 23, 818-823 (2017).
36. Zelezetsky, I. & Tossi, A. Alpha-helical antimicrobial peptides-Using a sequence template to guide structure-activity relationship studies. Biochim. Biophys. Acta—Biomembr. 1758, 1436-1449 (2006).
37. Porto, W. F. et al. In silico optimization of a guava antimicrobial peptide enables combinatorial exploration for peptide design. Nat. Commun. 9, 1490 (2018).
38. Buck, M. Trifluoroethanol and colleagues: Cosolvents come of age. Recent studies with peptides and proteins. Q. Rev. Biophys. 31, 297-355 (1998).
39. Luo, P. & Baldwin, R. L. Mechanism of helix induction by trifluoroethanol: A framework for extrapolating the helix-forming properties of peptides from trifluoroethanol/water mixtures back to water. Biochemistry 36, 8413-8421 (1997).
40. Nick Pace, C. & Martin Scholtz, J. A Helix Propensity Scale Based on Experimental Studies of Peptides and Proteins. Biophys. J. 75, 422-427 (1998).
41. Lindahl, E., Hess, B. & van der Spoel, D. GROMACS 3.0: a package for molecular simulation and trajectory analysis. Mol. Model. Annu. 7, 306-317 (2001).
42. Abraham, M. J. et al. GROMACS: High performance molecular simulations through multi-level parallelism from laptops to supercomputers. SoftwareX 1-2, 19-25 (2015).
43. Mendes, M. A., Souza, B. M., Marques, M. R. & Palma, M. S. Structural and biological characterization of two novel peptides from the venom of the neotropical social wasp Agelaia pallipes pallipes. Toxicon 44, 67-74 (2004).
44. Cutrona, K. J., Kaufman, B. A., Figueroa, D. M. & Elmore, D. E. Role of arginine and lysine in the antimicrobial mechanism of histone-derived antimicrobial peptides. FEBS Lett. 589, 3915-3920 (2015).
45. Eisenberg, D. Three-dimensional structure of membrane and surface proteins. Ann. Rev. Biochem. 53, 595-623 (1984).
46. Jin, L. et al. A Designed Tryptophan- and Lysine/Arginine-Rich Antimicrobial Peptide with Therapeutic Potential for Clinical Antibiotic-Resistant Candida albicans Vaginitis. J. Med. Chem. 59, 1791-1799 (2016).
47. Epand, R. M. & Epand, R. F. Lipid domains in bacterial membranes and the action of antimicrobial agents. Biochim. Biophys. Acta—Biomembr. 1788, 289-294 (2009).
48. Chongsiriwatana, N. P. & Barron, A. E. Comparing Bacterial Membrane Interactions of Antimicrobial Peptides and Their Mimics BT—Antimicrobial Peptides: Methods and Protocols. in (eds. Giuliani, A. & Rinaldi, A. C.) 171-182 (Humana Press, 2010). doi:10.1007/978-1-60761-594-1_12
49. De Kruijff, B., Killian, J. A., Rietveld, A. G. & Kusters, R. Chapter 13 Phospholipid Structure and Escherichia Coli Membranes. in Lipid Polymorphism and Membrane Properties (ed. Epand, R. M. B. T.-C. T. in M.) 44, 477-515 (Academic Press, 1997).
50. Chou, H.-T., Wen, H.-W., Kuo, T.-Y., Lin, C.-C. & Chen, W.-J. Interaction of cationic antimicrobial peptides with phospholipid vesicles and their antibacterial activity. Peptides 31, 1811-1820 (2010).
51. Luo, P. & Baldwin, R. L. Mechanism of Helix Induction by Trifluoroethanol: A Framework for Extrapolating the Helix-Forming Properties of Peptides from Trifluoroethanol/Water Mixtures Back to Water. Biochemistry 36, 8413-8421 (1997).
52. Radhakrishnan, M., Ganesh, S., L., P. P. & Padmanabhan, B. Circular Dichroism of Designed Peptide Helices and β-Hairpins: Analysis of Trp- and Tyr-Rich Peptides. ChemBioChem 6, 2152-2158 (2005).
53. Pacor, S., Giangaspero, A., Bacac, M., Sava, G. & Tossi, A. Analysis of the cytotoxicity of synthetic antimicrobial peptides on mouse leucocytes: implications for systemic use. J. Antimicrob. Chemother. 50, 339-348 (2002).
54. Jin, Y. et al. Antimicrobial Activities and Structures of Two Linear Cationic Peptide Families with Various Amphipathic β-Sheet and α-Helical Potentials. Antimicrob. Agents Chemother. 49, 4957-4964 (2005).
55. Lohner, K. The role of membrane lipid composition in cell targeting and their mechanism of action. in Developmentof novel antimicrobial agents, emerging strategies (ed. Lohner, K.) 149-165 (Norfolk: Horizon Scientific Press, 2001).
56. Henriksen, J. et al. Universal Behavior of Membranes with Sterols. Biophys. J. 90, 1639-1649 (2006).
57. Nagaraj, N. S. and R. Host-defense Antimicrobial Peptides: Importance of Structure for Activity. Current Pharmaceutical Design 8, 727-742 (2002).
58. Yeaman, M. R. & Yount, N. Y. Mechanisms of Antimicrobial Peptide Action and Resistance. Pharmacol. Rev. 55, 27 LP-55 (2003).
59. Seo, M.-D., Won, H.-S., Kim, J.-H., Mishig-Ochir, T. & Lee, B.-J. Antimicrobial Peptides for Therapeutic Applications: A Review. Molecules 17, 12276-12286 (2012).
60. Diao, L. & Meibohm, B. Pharmacokinetics and pharmacokinetic-pharmacodynamic correlations of therapeutic peptides. Clin. Pharmacokinet. 52, 855-868 (2013).
61. Villegas, V., Viguera, A. R., Aviles, F. X. & Serrano, L. Stabilization of proteins by rational design of α-helix stability using helix/coil transition theory. Fold. Des. 1, 29-34 (1996).
62. B., van W. S., L., P. S. & J., J. K. Half-Life Extension of Biopharmaceuticals using Chemical Methods: Alternatives to PEGylation. ChemMedChem 11, 2474-2495 (2016).
63. Aston, W. J. et al. A systematic investigation of the maximum tolerated dose of cytotoxic chemotherapy with and without supportive care in mice. BMC Cancer 17, 684 (2017).
64. Zhang, Q., Zeng, S. X. & Lu, H. Determination of maximum tolerated dose and toxicity of Inauhzin in mice. Toxicol. Reports 2, 546-554 (2015).
65. Lobo, E. D. & Balthasar, J. P. Pharmacokinetic—pharmacodynamic modeling of methotrexate-induced toxicity in mice. J. Pharm. Sci. 92, 1654-1664 (2003).
66. Hassan, F. et al. A mouse model study of toxicity and biodistribution of a replication defective adenovirus serotype 5 virus with its genome engineered to contain a decoy hyper binding site to sequester and suppress oncogenic HMGA1 as a new cancer treatment therapy. PLoS One 13, e0192882 (2018).
67. Mulder, K. C. L., Viana, A. A. B. & Parachin, M. X. and N. S. Critical Aspects to be Considered Prior to Large-Scale Production of Peptides. Current Protein & Peptide Science 14, 556-567 (2013).
68. Bradshaw, J. P. Issues for Potential Clinical Use. 17, 233-240 (2003).
69. da Costa, J. P., Cova, M., Ferreira, R. & Vitorino, R. Antimicrobial peptides: an alternative for innovative medicines? Appl. Microbiol. Biotechnol. 99, 2023-2040 (2015).
70. Fjell, C. D., Hiss, J. A., Hancock, R. E. W. & Schneider, G. Designing antimicrobial peptides: form follows function. Nat. Rev. Drug Discov. 11, (2011).
71. Li, Y. Recombinant production of antimicrobial peptides in Escherichia coli: A review. Protein Expr. Purif. 80, 260-267 (2011).
72. Ong, Z. Y., Wiradharma, N. & Yang, Y. Y. Strategies employed in the design and optimization of synthetic antimicrobial peptide amphiphiles with enhanced therapeutic potentials. Adv. Drug Deliv. Rev. 78, 28-45 (2014).
73. Zhao, C. X. et al. A simple and low-cost platform technology for producing pexiganan antimicrobial peptide in E. coli. Biotechnol. Bioeng. 112, 957-964 (2015).
74. Nagashima, K. et al. Role of lysine residue at 7th position of wasp chemotactic peptides. Biochem. Biophys. Res. Commun. 168, 844-849 (1990).
75. Taniguchi, M. et al. Effect of substituting arginine and lysine with alanine on antimicrobial activity and the mechanism of action of a cationic dodecapeptide (CL(14-25)), a partial sequence of cyanate lyase from rice. Biopolymers 102, 58-68 (2014).
76. Lee, J.-K., Park, S.-C., Hahm, K.-S. & Park, Y. Antimicrobial HPA3NT3 peptide analogs: Placement of aromatic rings and positive charges are key determinants for cell selectivity and mechanism of action. Biochim. Biophys. Acta—Biomembr. 1828, 443-454 (2013).
77. Du, Q. et al. Cationicity-Enhanced Analogues of the Antimicrobial Peptides, AcrAP1 and AcrAP2, from the Venom of the Scorpion, Androctonus crassicauda, Display Potent Growth Modulation Effects on Human Cancer Cell Lines. Int. J. Biol. Sci. 10, 1097-1107 (2014).
78. El Solh, A. A. et al. Persistent Infection with Pseudomonas aeruginosa in Ventilator-associated Pneumonia. Am. J. Respir. Crit. Care Med. 178,513-519 (2008).
79. Newman, J. W., Floyd, R. V & Fothergill, J. L. The contribution of Pseudomonas aeruginosa virulence factors and host factors in the establishment of urinary tract infections. FEMS Microbiol. Lett. 364, fnx124-fnx124 (2017).
80. Yeung, C. K. & Lee, K. H. Community acquired fulminant Pseudomonas infection of the gastrointestinal tract in previously healthy infants. J. Paediatr. Child Health 34, 584-587 (1998).
81. Nagoba, B. et al. Treatment of skin and soft tissue infections caused by Pseudomonas aeruginosa—A review of our experiences with citric acid over the past 20 years. Wound Med. 19,5-9 (2017).
82. Dryden, M. S. Complicated skin and soft tissue infection. J. Antimicrob. Chemother. 65, iii35-iii44 (2010).
83. Buivydas, A. et al. Clinical isolates of Pseudomonas aeruginosa from superficial skin infections have different physiological patterns. FEMS Microbiol. Lett. 343,183-189 (2013).
84. Stefani, S. et al. Relevance of multidrug-resistant Pseudomonas aeruginosa infections in cystic fibrosis. Int. J. Med. Microbiol. 307,353-362 (2017).
85. Chen, C., Mangoni, M. L. & Di, Y. P. In vivo therapeutic efficacy of frog skin-derived peptides against Pseudomonas aeruginosa-induced pulmonary infection. Sci. Rep. 7,8548 (2017).
86. Wiegand, I., Hilpert, K. & Hancock, R. E. W. Agar and broth dilution methods to determine the minimal inhibitory concentration (MIC) of antimicrobial substances. Nat. Protoc. 3,163-175 (2008).
87. de la Fuente-Núñez, C. et al. Inhibition of Bacterial Biofilm Formation and Swarming Motility by a Small Synthetic Cationic Peptide. Antimicrob. Agents Chemother. 56, 2696-2704 (2012).
88. Shalel, S., Streichman, S. & Marmur, A. The mechanism of hemolysis by surfactants: effect of solution composition. J. Colloid Interface Sci. 252,66-76 (2002).
89. Love, L. The hemolysis of human erythrocytes by sodium dodecyl sulfate. J. Cell. Comp. Physiol. 36,133-148 (1950).
90. Powell, M. F. et al. Peptide Stability in Drug Development. II. Effect of Single Amino Acid Substitution and Glycosylation on peptide Reactivity in Human Serum. Pharm. Res. 10,1268-1273 (1993).
91. Akey, D. L. et al. A New Structural Form in the SAM/Metal-Dependent O-Methyltransferase Family: MycE from the Mycinamicin Biosynthetic Pathway. J. Mol. Biol. 413,438-450 (2011).
92. Fiser, A. & Šali, A. B. T.-M. in E. Modeller: Generation and Refinement of

Homology-Based Protein Structure Models. Macromolecular Crystallography, Part D 374,461-491 (Academic Press, 2003).
93. Laskowski, R. A., MacArthur, M. W., Moss, D. S. & Thornton, J. M. PROCHECK: a program to check the stereochemical quality of protein structures. J. Appl. Crystallogr. 26,283-291 (1993).
94. Wiederstein, M. & Sippl, M. J. ProSA-web: interactive web service for the recognition of errors in three-dimensional structures of proteins. Nucleic Acids Res. 35, W407-W410 (2007).

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are cjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc. A

sed herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

What is claimed is:

1. An antimicrobial peptide comprising the amino acid sequence of any one of SEQ ID NOs: 2-383.

2. The antimicrobial peptide of claim 1, wherein the antimicrobial peptide comprises the amino acid sequence of SEQ ID NO: 8, SEQ ID NO: 16, SEQ ID NO: 17, SEQ ID NO: 18, or SEQ ID NO: 21.

3. The antimicrobial peptide of claim 1, wherein the antimicrobial peptide consists of the amino acid sequence of any one of SEQ ID NOs: 2-383.

4. The antimicrobial peptide of claim 3, wherein the antimicrobial peptide consists of the amino acid sequence of SEQ ID NO: 8, SEQ ID NO: 16, SEQ ID NO: 17, SEQ ID NO: 18, or SEQ ID NO: 21.

5. A composition comprising the antimicrobial peptide of any one of claims 1-4, optionally further comprising a pharmaceutically acceptable carrier.

6. A method of treating a microbial infection comprising administering, to a subject in need of such treatment, a therapeutically effective amount of the antimicrobial peptide of any one of claims 1-4 or the composition of claim 5.

7. The method of claim 6, wherein the subject is a mammal.

8. The method of claim 6 or claim 7, wherein the subject is human.

9. The method of any one of claims 6-8, wherein the antimicrobial peptide or the composition is administered orally, intravenously, intramuscularly, subcutaneously, or topically.

10. The method of any one of claims 6-9, wherein the microbial infection comprises a bacterial, fungal, algal, viral, or protozoan infection.

11. The method of claim 10, wherein the microbial infection comprises a bacterial infection, wherein the bacterial infection is a Gram-positive bacterial infection.

12. The method of claim 11, wherein the bacterial infection comprises a Gram-positive bacterium selected from the group consisting of a Micrococcus luteus bacterium, a Staphylococcus aureus bacterium, a Staphylococcus epidermidis bacterium, a Bacillus megaterium bacterium, and an Enterococcus faecium bacterium.

13. The method of claim 12, wherein:
(a) the bacterium is a Micrococcus luteus bacterium, and wherein the Micrococcus luteus bacterium is strain A270;
(b) the bacterium is a Staphylococcus aureus bacterium, and wherein the Staphylococcus aureus bacterium is strain ATCC29213 or ATCC12600;
(c) the bacterium is a Staphylococcus epidermidis bacterium, and wherein the Staphylococcus epidermidis bacterium is a strain ATCC12228; or
(d) the bacterium is a Bacillus megaterium bacterium, and wherein the Bacillus megaterium bacterium is a strain ATCC10778.

14. The method of any one of claims 10-14, wherein the microbial infection comprises a bacterial infection, wherein the bacterial infection is a Gram-negative bacterial infection.

15. The method of claim 14, wherein the bacterial infection comprises a bacterium selected from the group consisting of an Escherichia coli bacterium, an Enterobacter cloacae bacterium, a Serratia marcescens bacterium, a Pseudomonas aeruginosa bacterium, a Klebsiella pneumoniae bacterium, and an Acinetobacter baumannii bacterium.

16. The method of claim 15, wherein:
(a) the bacterium is an Escherichia coli bacterium, and wherein the Escherichia coli bacterium is a strain SBS 363 or BL21;
(b) the bacterium is an Enterobacter cloacae bacterium, and wherein the Enterobacter cloacae bacterium is a strain ®-12;
(c) the bacterium is a Serratia marcescens bacterium, and wherein the Serratia marcescens bacterium is a strain ATCC4112; or
(d) the bacterium is a Pseudomonas aeruginosa bacterium, and wherein the Pseudomonas aeruginosa bacterium is a strain PA14 or PA01.

17. The method of any one of claims 10-16, wherein the microbial infection comprises a fungal infection, wherein the fungal infection comprises a pathogenic yeast.

18. The method of claim 17, wherein the pathogenic yeast is selected from the group consisting of Candida albicans and Candida tropicalis.

19. The method of claim 18, wherein:
(a) the yeast is Candida albicans, wherein the Candida albicans is strain MDM8; or
(b) and yeast is Candida tropicalis, wherein the Candida tropicalis is strain IOC4560.
